(19) United States
(12) Patent Application Publication
(10) Pub. No.: US 2026/0130770 A1
(43) Pub. Date: May 14, 2026

(54) SPINAL IMPLANT SENSOR ASSEMBLY

(71) Applicant: CANARY MEDICAL SWITZERLAND AG, Baar (CH)

(72) Inventors: Kevin Gemmell, San Diego, CA (US); Jeffrey M. Gross, Carlsbad, CA (US); Peter J. Schiller, Shakopee, MN (US); Mark A. Adler, Carlsbad, CA (US); Michael Johnson, San Marcos, CA (US); Steven Cazarez, San Diego, CA (US)

(21) Appl. No.: 19/118,691

(22) PCT Filed: Oct. 5, 2023
(86) PCT No.: PCT/US2023/034585

§ 371 Date: Apr. 4, 2025

Related U.S. Application Data
(60) Provisional application No. 63/378,588, filed on Oct. 6, 2022.

(51) Int. Cl. A61F 2/44; A61B 5/00; A61F 2/30

(52) U.S. Cl. CPC A61F 2/4455; A61B 5/686; A61B 2560/0214; A61B 2562/0219; A61F 2002/30593; A61F 2002/3067

(57) ABSTRACT

An implantable sensor assembly for use during spinal fusion. The implantable sensor assembly can include a component of an implantable prosthesis and an implantable cartridge associated with the component. The implantable cartridge can include at least one sensor capable of detecting one or more kinematic measurements associated with a patient and generating sensor data, and an antenna in electrical communication with at least one sensor, wherein the antenna transmits sensor data to a receiver at a remote location.

### CROSS-REFERENCE TO RELATED APPLICATIONS

All applications for which a foreign or domestic priority claim is identified in the Application Data Sheet as filed with the present application are hereby incorporated by reference.

### BACKGROUND

### Field

The present disclosure relates generally to intelligent implants associated with implantable systems such as spinal implants, and more particularly, to intelligent implants with implantable reporting processors that sample, record, and transmit information related to the placement and integrity of an implanted system, and the health of the patient in which the system is implanted, as well as features of intelligent implants including enhanced transmitting antenna configurations and data sampling methods.

### Description of the Related Art

Spinal implants may be configured to connect two or more vertebrae of the spine. The spinal fusion can occur at any portion of the spine (e.g., lumbar spine, cervical spine, thoracic spine). Systems intended to permanently connect two or more vertebrae to eliminate motion between them are referred to as spinal fusion that involves techniques designed to form bridging bone between vertebrae as part of the healing process.

Currently, there is no mechanism for reliably detecting misplacement, instability, or misalignment in the spinal implant without clinical visits and the hands and visual observations of an experienced health care provider. Even then, early identification of subclinical problems or conditions is either difficult or impossible since they are often too subtle to be detected on physical exam or demonstrable by radiographic studies. Furthermore, even if detection were possible, corrective actions would be hampered by the fact that the specific amount of movement and/or degree of improper alignment cannot be accurately measured or quantified, making targeted, successful intervention unlikely. Existing external monitoring devices do not provide the fidelity required to detect instability since these devices are separated from the spinal implant by skin, muscle, and fat—each of which masks the mechanical signatures of instability and introduce anomalies such as flexure, tissue-borne acoustic noise, inconsistent sensor placement on the surface, and inconsistent location of the external sensor relative to the spinal implant.

In addition, a patient may experience several complications post-procedure. Such complications include neurological symptoms, pain, malfunction (blockage, loosening, etc.) and/or wear of the implant, movement or breakage of the implant, inflammation and/or infection. While some of these problems can be addressed with pharmaceutical products and/or further surgery, they are difficult to predict and prevent; often early identification of complications and side effects, although desirable, is difficult or impossible.

The present disclosure is directed to intelligent implants with implantable reporting processors that sample, record, and transmit information related to the placement and integrity of a spinal implant, and the health of the patient in which the spinal implant is implanted, as well as intelligent implants having enhanced transmitting antenna configurations and data sampling methods.

### SUMMARY

In some aspects, an implantable sensor assembly is disclosed for use during spinal fusion or other spinal procedures. The implantable sensor assembly can include a component and an implantable cartridge associated with the component. The component can form a part of an implantable prosthesis. The implantable cartridge can include at least one sensor capable of detecting one or more kinematic measurements associated with a patient and generating sensor data. The implantable cartridge can also include an antenna in electrical communication with the sensor. The antenna can transmit sensor data to a receiver at a remote location.

The implantable sensor assembly can include one or more of the following features. The implantable cartridge can include a battery. The implantable cartridge can include an inertial measurement unit having a plurality of accelerometers and/or a plurality of gyroscopes. For example, the inertial measurement unit can include a first accelerometer and/or a first gyroscope for measuring data associated with a first measurement axis. The inertial measurement unit can also include a second accelerometer and/or a second gyroscope for measuring data associated with a second measurement axis. The inertial measurement unit can also include a third accelerometer and/or a third gyroscope for measuring data associated with a third measurement axis. In some configurations, the implantable cartridge can have two or more distinct inertial measurement units for use in separate assessments, for example separate kinematic functionality. In some configurations, the implantable cartridge can have one or more accelerometers and/or gyroscopes separate from the inertial measurement unit.

In certain aspects, the implantable cartridge can include a length in conjunction with a circular, oval, square, or rectangular cross section. The implantable cartridge can have a cross-section with a corner radius associated with a square or rectangular cross-section. The implantable cartridge can be interface with or be positioned within a spinal implant. In some embodiments, the spinal implant can be, an interbody spacer or a spinal cage. For example, the implantable cartridge can be insertable into an opening of the interbody spacer or into a slot of the cage. The cartridge can be mechanically coupled to the component. The cartridge can be reversibly coupled to the component. The cartridge can form a one-way positive connection with the component. The cartridge can be mechanically coupled to the component using at least one of corresponding snap rings, locks, twists, threads, or chemical adhesives. The cartridge can be press-fit into the component. The cartridge can include a plurality of sensors positioned on a top surface of the cartridge. The cartridge can include a plurality of sensors positioned on a bottom surface of the cartridge. The cartridge can include at least one sensor positioned on a proximal surface of the cartridge. The cartridge can include at least one sensor positioned on a distal surface of the cartridge. The plurality of sensors can be positioned on the top surface of the cartridge and the plurality of sensors positioned on the bottom surface of the cartridge measure subsidence of the implantable sensor assembly. At least one sensor can be positioned on a proximal surface of the cartridge and a distal surface of the cartridge provide translational and orientation related movements of the implantable sensor assembly. The plurality of sensors can be positioned on the top surface of the cartridge are in series. The plurality of sensors positioned on the bottom surface of the cartridge are in series. The cartridge can comprise a series of three sensors on a top surface of the cartridge, a series of three sensors on a bottom surface of the cartridge, a plurality of sensors on a proximal end of the cartridge, and a plurality of sensors on a distal end of the cartridge. The antenna can extend from a proximal end or a distal end of the cartridge. The antenna can be contained within a gap of the component. The antenna can be contained within the component whose material composition enables signal transmission from the antenna. The component can comprise Polyether Ether Ketone (PEEK). The implantable sensor assembly can further include a processor. The component of the implantable prosthesis can be an interbody spacer or a cage for use during spinal fusion. The interbody spacer can be inserted into the lumbar region of the spine. The interbody spacer can be inserted into the cervical region of the spine. The interbody spacer can be inserted into the thoracic region of the spine. The cage can be a lumbar cage. The cage can be a cervical cage. The cage can be a thoracic cage. The implantable sensor can further include at least one sensor can be an ultrasonic sensor. The ultrasonic sensor can be an M-mode sensor. The ultrasonic sensor can be a B-mode sensor. The ultrasonic sensor can be a low-power sensor. The antenna can be a loop antenna. The antenna can be a conformal antenna. The antenna can continuously transmit sensor data. The antenna can intermittently transmit sensor data. At least one sensor can continuously detect one or more physiological parameters. At least one sensor can intermittently detect one or more physiological parameters. The power source can provide power to at least one sensor. The power source can be rechargeable. At least one sensor can be capable of being powered by a power source outside a body of the patient. The implantable sensor can further include a memory device for storing data from at least one sensor. The memory device can have sufficient memory to enable firmware upgrades of the sensor assembly.

In some aspects, a spinal implant assembly is disclosed for use during spinal fusion or other spinal procedures. A cartridge can be mechanically coupled to the interbody spacer or the spinal cage. The cartridge can include at least one sensor and an antenna. At least one sensor can be capable of detecting one or more physiological parameters of a patient and generating sensor data. The antenna can be in electrical communication with at least one sensor and provide bi-directional data communication to a receiver at a remote location. The cartridge can be insertable into a mating female cavity within the interbody spacer or the spinal cage.

The spinal implant assembly can include one or more of the following features. The spinal implant assembly can include a battery. The spinal implant assembly can include an inertial measurement unit having a plurality of accelerometers and a plurality of gyroscopes. The inertial measurement unit can include a first accelerometer and a first gyroscope for measuring data associated with a first measurement axis. The inertial measurement unit can include a second accelerometer and a second gyroscope for measuring data associated with a second measurement axis. The inertial measurement unit can include a third accelerometer and a third gyroscope for measuring data associated with a third measurement axis. The inertial measurement unit can include a tilt sensor. The inertial measurement unit can include a strain sensor or other type of sensor that measures and/or is responsive to deflection. The inertial measurement unit can include one or more of an accelerometer, a gyroscope, a tilt sensor and a strain sensor. For example, the inertial measurement unit can include an accelerometer and a gyroscope and a tilt sensor. The cartridge can have a length in conjunction with a circular, oval, square, or rectangular cross section. The cartridge can have a cross-section with a corner radius associated with a square or rectangular cross-section. The cartridge can be mechanically coupled to the interbody spacer or the spinal cage. The cartridge can be reversibly coupled to the interbody spacer or the spinal cage. The cartridge can form a one-way positive connection with the interbody spacer or the spinal cage. The cartridge can be mechanically coupled to the interbody spacer or the spinal cage using at least one of corresponding snap rings, locks, twists, threads, or a chemical adhesive. The cartridge can be press-fit into the interbody spacer or the spinal cage. The cartridge can include a plurality of sensors positioned on a top surface of the cartridge. The cartridge can include a plurality of sensors positioned on a bottom surface of the cartridge. The cartridge can include at least one sensor positioned on a proximal surface of the cartridge. The cartridge can include at least one sensor positioned on a distal surface of the cartridge. The plurality of sensors positioned on the top surface of the cartridge and the plurality of sensors positioned on the bottom surface of the cartridge can be configured to measure subsidence of the interbody spacer or the spinal cage. At least one sensor can be on a proximal surface of the cartridge and a distal surface of the cartridge provide translational and orientation related movements of the interbody spacer or the spinal cage. The plurality of sensors positioned on the top surface of the cartridge can be in series. The plurality of sensors positioned on the bottoms bottom surface of the cartridge are in series. The cartridge can include a series of three sensors on a top surface of the cartridge, a series of three sensors on a bottom surface of the cartridge, a plurality of sensors on a proximal end of the cartridge, and a plurality of sensors on a distal end of the cartridge. The antenna can extend from a proximal end or a distal end of the cartridge. The spinal implant assembly can include a processor. The spinal implant assembly can be inserted into a portion of a lumbar spine of a patient. The spinal page can be inserted into a portion of a cervical spine of a patient. The interbody spacer or the spinal cage can be inserted into a portion of a thoracic spine of a patient. At least one sensor can be an ultrasonic sensor. The ultrasonic sensor can be an M-mode sensor. The ultrasonic sensor can be a B-mode sensor. The ultrasonic sensor can be a low-power sensor. The antenna can be a loop antenna. The antenna can be a conformal antenna. In one embodiment the antenna provides data transfer. The antenna can continuously transmit sensor data. The antenna can intermittently transmit sensor data. At least one sensor can continuously detect one or more physiological parameters. At least one sensor can intermittently detect one or more physiological parameters. The spinal implant assembly can include a power source for providing power to at least one sensor. The power source can be rechargeable. At least one sensor can be powered by a power source outside a body of a patient. Ine one embodiment the antenna can be an inductive power receiver for secondary capacitance or battery charging. The spinal implant assembly can include a memory device for storing data from at least one sensor.

In some aspects, a method of sampling data from an implantable cartridge coupled to an interbody spacer or a spinal cage implanted in a patient is disclosed. The method can include detecting one or more kinematic measurements associated with a patient's movements and generating sensor data. The method can include transmitting sensor data to and receiving data from a receiver at a remote location.

The method of sampling data can include one or more of the following steps and features. The method can include detecting one or more kinematic measurements occurs during movement of the patient. The method can include detecting one or more kinematic measurements that occurs when the interbody spacer or the spinal cage is under load. The method can include calibrating the implantable cartridge when the patient is at a known position. The known position can be when the patient is laying down. The known position can be when the patient is standing against a wall. The known position can be when a patient's back is at a predetermined angle while the patient is in a sitting position. The predetermined angle can be 30 degrees, 45 degrees, or 90 degrees. The one or more kinematic measurements can be used to determine fusion of the interbody spacer or the spinal cage. The one or more kinematic measurements can be used to determine subsidence of the interbody spacer or the spinal cage. The one or more kinematic measurements can be used to determine migration of the interbody spacer or the spinal cage. The migration of the interbody spacer or the spinal cage can be used to measure translation of the interbody spacer or the spinal cage at a point of implantation. The one or more kinematic measurements can be used to determine patient movement. The determined patient movement can include, for example, one of step count, cadence, walking speed, angle of motion, and gait. The method can be configured to determine how quickly the patient can return to regular activities. The implantable cartridge of the method can include at least one sensor capable of detecting one or more kinematic measurements associated with a patient and generating sensor data. The implantable cartridge can include an antenna in electrical communication with at least one sensor. The antenna can transmit sensor data to and receives data from a receiver at a remote location. The implantable cartridge can include a battery. The method can include detecting one or more kinematic measurements comprising obtaining the one or more kinematic measurements from an inertial measurement unit having a plurality of accelerometers and/or a plurality of gyroscopes. The method can include detecting tilt using a tilt sensor. The tilt sensor may be used to detect angular movement of the implant. The method can include detecting deflection on the implant by use of a strain sensor. The method can include measuring data associated with a first measurement axis, wherein the data associated with the first measurement axis is obtained from a first accelerometer and a first gyroscope of the inertial measurement unit. The method can include measuring data associated with a second measurement axis, wherein the data associated with the second measurement axis is obtained from a second accelerometer and a second gyroscope of the inertial measurement unit. The method can include measuring data associated with a third measurement axis, wherein the data associated with the third measurement axis is obtained from a third accelerometer and a third gyroscope of the inertial measurement unit. Optionally, the measured data is obtained from a tilt sensor. Optionally, the measured data is obtained from a strain sensor.

In some aspects, a spinal implant assembly is disclosed for use during spinal fusion. The spinal implant assembly can include a spinal implant. The spinal implant can include a body. The spinal implant can also include an opening on a first end of the body. The spinal implant can include a cavity extending through the body of the spinal implant from the opening towards a second end side of the body. The spinal implant can include a cartridge configured to be inserted into the cavity, wherein the cartridge comprises an outer wall configured to house a plurality of components.

The spinal implant can include one or more of the following features. The spinal implant can include a locking structure for securing the spinal implant with the cartridge. The spinal implant can include at least one of a top surface of the body or a bottom surface of the body comprises a plurality of ridges, wherein the plurality of ridges is configured to improve engagement of the spinal implant with an adjacent vertebrae. The spinal implant can include a hole extending from a top surface of the body to a bottom surface of the body. In certain aspects, the hole of the spinal implant is configured to be filled with biological or synthetic material to aid in spinal fusion. In certain aspects, the spinal implant is an interbody spacer or a spinal cage.

In certain aspects, the cartridge can include a power source. The cartridge can include a processor. The cartridge can include memory device for storing data from the at least one sensor. The power source can include either a single use or a rechargeable battery. The cartridge can include an antenna. In certain aspects, the antenna can be positioned on the cartridge within the opening of the spinal implant. For example, the antenna can be positioned within the outer wall of the cartridge. In certain aspects, the antenna is at least one of a loop antenna and a conformal antenna. In some embodiments, the antenna continuously transmits sensor data. In some examples, the antenna intermittently transmits sensor data. In certain aspects, the cartridge can include at least one sensor. In some examples, the at least one sensor is an ultrasonic sensor. In some embodiments, the ultrasonic sensor is a lower-power sensor. In some examples, the at least one sensor continuously or intermittently detects one or more physiological parameters.

The cartridge can have one or more of the following features. In some embodiments, the cartridge can include a length in conjunction with a circular, oval, square, or rectangular cross-section. In some embodiments, the outer wall has a wall thickness about 0.5 mm. The outer wall can have a wall thickness less than 1 mm. The cartridge can have a width to height ratio of 1:2. The cartridge can have a width to height ratio of 2:3. The cartridge can have a width of 8 mm, a thickness of 4 mm, and a length of 28 mm. The cartridge can be reversibly coupled to the spinal implant.

In some embodiments, the spinal implant can include a locking structure. In some embodiments, the locking structure can include a locking spring finger configured to deform outward on the spinal implant as the cartridge is inserted and is configured to move back into place once the cartridge is fully inserted. In some examples, the locking structure can include a pin positioned on the spinal implant and a locking ledge positioned on the cartridge, wherein the pin on the spinal implant is configured to retain the locking ledge to retain the cartridge after insertion. In some embodiments, the locking ledge has a radius of the pin. In some embodiments, the locking structure can include a clip or groove positioned along a length of the cartridge, wherein the clip or groove is configured to retain the cartridge within the spinal implant.

In certain aspects, an intelligent implant assembly is disclosed for implantation within a patient. The intelligent implant assembly can include an implant body and a cartridge. In some embodiments, the implant body can include an opening on a first end of the implant body. The implant body can include a cavity extending through the implant body from the opening towards a second side of the implant body. In some examples, the cartridge can be inserted into the intelligent implant, wherein the cartridge is retained within the cavity of the implant body and within an outer perimeter of the implant body. In some embodiments, the cartridge can include an outer wall configured to house a plurality of components. In certain aspects, the intelligent implant assembly can include a locking structure for securing the spinal implant with the spinal implant. In some aspects, at least one of a left side surface of the body or a right side surface of the implant body comprises a plurality of ridges, wherein the plurality of ridges is configured to improve engagement of the spinal implant with an adjacent vertebrae.

The cartridge can have one or more of the following features. In some examples, the cartridge includes a power source. The power source can include either a single use or a rechargeable battery. The cartridge can include an antenna. The antenna can be positioned on the cartridge within the opening of the spinal implant. The antenna can be positioned within the outer wall of the cartridge. The antenna can be at least one of a loop antenna and a conformal antenna. The antenna can continuously transmit sensor data. The antenna can intermittently transmit sensor data. In some embodiments, the cartridge comprises a processor. In some examples, the cartridge comprises at least one sensor. The at least one sensor can be an ultrasonic sensor. The ultrasonic sensor can be a lower-power sensor. The at least one sensor can continuously or intermittently detect one or more physiological parameters. In some embodiments, the cartridge can include a memory device for storing data from the at least one sensor. The cartridge can have a length in conjunction with a circular, oval, square, or rectangular cross-section. In some embodiments, the cartridge has an outer wall with a wall thickness about 0.5 mm. The outer wall can have a wall thickness less than 1 mm. In some examples, the cartridge has a width to height ratio of 1:2. In some embodiments the cartridge has a width to height ratio of 2:3. The cartridge can be reversibly coupled to the spinal implant.

The locking structure can have one or more of the following features. For example, the locking structure comprises a locking spring finger configured to deform outward on the spinal implant as the cartridge is inserted and is configured to move back into place once the cartridge is fully inserted. In some examples, the locking structure comprises a pin positioned on the spinal implant and a locking ledge positioned on the cartridge, wherein the pin on the spinal implant is configured to retain the locking ledge to retain the cartridge after insertion. In some embodiments, the locking ledge has a radius of the pin. The locking structure can include a clip or groove positioned along a length of the cartridge, wherein the clip or groove is configured to retain the cartridge within the spinal implant.

In some embodiments, disclosed is a method of monitoring patient recovery after spinal fusion. The method can include providing a spinal implant assembly comprising a spinal implant and a cartridge, the cartridge comprising at least one sensor. In some examples, the method can include collecting data, by the at least one sensor, indicative of at least one of fusion, subsidence or migration of the spinal implant. In some embodiments, the method can include transmitting the data to a remote location. In some embodiments, the data comprises kinematic measurements of the patient's movements. The data can be indicative of movement of the spinal implant.

The spinal implant can comprise an opening on a first end of the spinal implant and a cavity, wherein the cavity extends through the body from a first end of the spinal implant to a second end of the spinal implant. The cartridge can be configured to be inserted into the opening of the spinal implant such that the cartridge is secured in the cavity of the spinal implant. In some embodiments, the cartridge comprises a power source, a memory source, and a processor. In some examples, one of the at least one sensor comprises an accelerometer and/or gyroscope. In some examples, one of the at least one sensor comprises an accelerometer and/or gyroscope and/or tilt sensor and/or strain sensor The accelerometer and/or gyroscope can be configured to measure at least one of a patient's step count, cadence, walking speed, angle of motion, or gait. In some embodiments, one of the at least one sensor comprises at least one ultrasound sensor. The at least one ultrasound sensor can be configured to detect translation of the spinal implant. In some embodiments, the translation of the spinal implant can be configured to measure migration of the spinal implant from a point of patient implantation. In some embodiments, the at least one ultrasound sensor is configured to measure a distance between a surface of the spinal implant and an adjacent vertebrae. In some examples, a change in distance between the surface of the spinal implant and the adjacent vertebrae is configured to measure subsidence of the spinal implant. In some embodiments, the at least one sensor comprises at least one tilt sensor. In some embodiments, the at least one sensor comprises at least one strain sensor or other sensor that measures deflection. The tilt sensor and/or strain sensor may be utilized to, e.g., detect angular movement. In some embodiments, the at least one sensor comprises at least one vibration sensor. The at least one vibration sensor can be configured to detect acoustic emissions associated with the spinal implant against an adjacent vertebrae. The acoustic emissions can be configured to measure fusion of the spinal implant against the adjacent vertebrae. In some embodiments, the at least one sensor is configured to calibrate the cartridge when the patient is at a known position. In some examples, the spinal implant is an interbody spacer or a spinal cage.

In certain aspects, a method for of monitoring patient recovery after spinal fusion is disclosed. The method can include receiving data from a spinal implant assembly. The method can include processing the data to evaluate migration of the spinal implant, subsidence of the spinal implant, and/or fusion of the spinal implant. The method can include providing an output to a clinician based on the processed data.

In some embodiments, the data can comprise kinematic measurements. The patient kinematic measurements can be associated with at least one of a patient's step count, cadence, walking speed, angle of motion, or gait. The data can comprise measurements indicative of migration of the spinal implant. In some embodiments, the method can include determining migration of the spinal implant based on translation of the spinal implant. In some embodiments, the data comprises measurements indicative of subsidence of the spinal implant. In some examples, the method further comprises determining subsidence of the spinal implant based on a change in the distance between a surface of the spinal implant and an adjacent vertebrae. In some examples, the data comprises measurements indicative of fusion of the spinal implant with adjacent vertebrae.

### BRIEF DESCRIPTION OF THE DRAWINGS

Exemplary features of the present disclosure, its nature and various advantages will be apparent from the accompanying drawings and the following detailed description of various embodiments. Non-limiting and non-exhaustive embodiments are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIG. 1A illustrates an implantable sensor assembly in the form of an interbody spacer with an insertable cartridge, wherein the cartridge includes an implantable reporting processor.

FIGS. 1B and 1C illustrate perspective views of the insertable cartridge of FIG. 1A.

FIG. 2A illustrates another implantable sensor assembly in the form of an interbody spacer with an insertable cartridge, wherein the cartridge includes implantable reporting processor.

FIGS. 2B, 2C and 2D illustrates a cross-sectional view of the insertable cartridge of FIG. 2A.

FIG. 3 illustrates an implantable sensor assembly in the form of an interbody spacer with an insertable cartridge, wherein the spinal cage includes a window.

FIG. 4A illustrates a cross-sectional medial-lateral view of the implantable sensor assembly of FIGS. 1A and 1B.

FIG. 4B illustrates a cross-sectional medial-lateral view of the implantable sensor assembly of FIGS. 2A and 2B.

FIG. 5A illustrates a front posterior-anterior view of an implantable sensor assembly with a spinal cage with an insertable cartridge, wherein the interbody spacer does not include a window.

FIG. 5B illustrates a front posterior-anterior view of an implantable sensor assembly with a spinal cage with an insertable cartridge, wherein the interbody spacer includes a window.

FIG. 6 illustrates the implantable sensor assembly of any of FIGS. 1A, 1B, 1C, 2A, 2B, 3, 4A, 4B, 5A and 5B implanted in the spine of a patient during spinal fusion.

FIG. 7 illustrates a context diagram of an intelligent implant environment in a patient's home.

FIG. 8 illustrates a block-diagram of an implantable circuit for an implantable sensor assembly, such as an implantable interbody spacer, where the circuit includes an implantable reporting processor (IRP).

FIG. 9 illustrates another block-diagram of an implantable circuit for an implantable sensor assembly, such as an implantable interbody spacer, where the circuit includes an implantable reporting processor (IRP).

FIG. 10 illustrates an inertial measurement unit (IMU) of the implantable reporting processor of FIGS. 8-9 and a set of coordinate axes within the frame of reference of the IMU.

FIGS. 11A, 11B, 11C, 11D and 11E illustrate various views of a loop antenna.

FIG. 12 illustrates a conformal antenna, according to certain aspects of the present disclosure.

FIG. 13 illustrates an embodiment of a planar inverted-F (PIFA) antenna, according to certain aspects of the present disclosure.

FIG. 14 illustrates an embodiment of a helix antenna.

FIG. 15 illustrates a block-diagram of an implantable circuit for an implantable sensor assembly, such as an implantable interbody spacer.

FIG. 16A illustrates a front posterior-anterior view of another embodiment of an implantable sensor assembly comprising an interbody spacer with an insertable cartridge.

FIG. 16B illustrates a rear posterior-anterior view of the implantable sensor assembly of FIG. 16A.

FIG. 16C illustrates a side view of another embodiment of the implantable sensor assembly of FIG. 16A.

FIGS. 16D and 16E illustrate a front perspective view and a rear perspective view respectively of the implantable sensor assembly of FIG. 16A.

FIG. 16F illustrates a top view of the implantable sensor assembly of FIG. 16A.

FIG. 16G illustrates a bottom view of the implantable sensor assembly of FIG. 16A.

FIG. 16H illustrates a cross sectional view of the implantable sensor assembly taken alone line A-A of FIG. 16F and the insertable cartridge positioned within an opening of the implantable sensor assembly.

FIG. 161 illustrates the cross-sectional view of the implantable sensor assembly of FIG. 16H with the insertable cartridge removed.

FIG. 17 illustrates a perspective view of another embodiment of an implantable sensor assembly with an implantable reporting processor inserted.

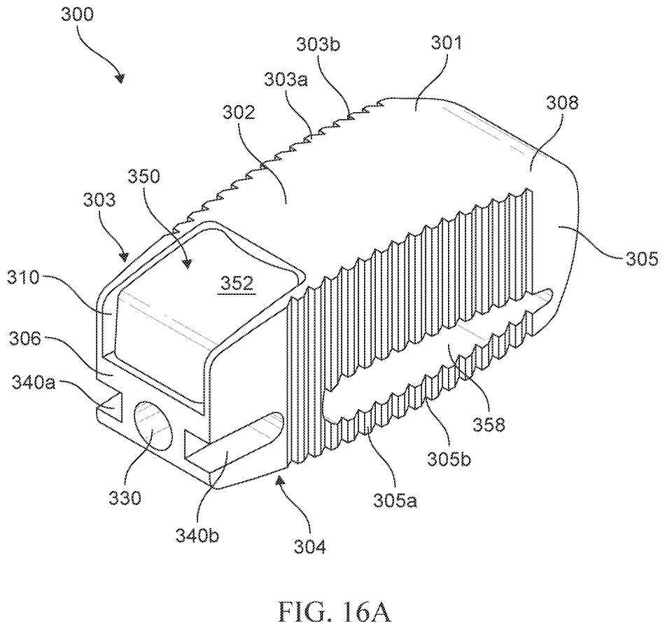

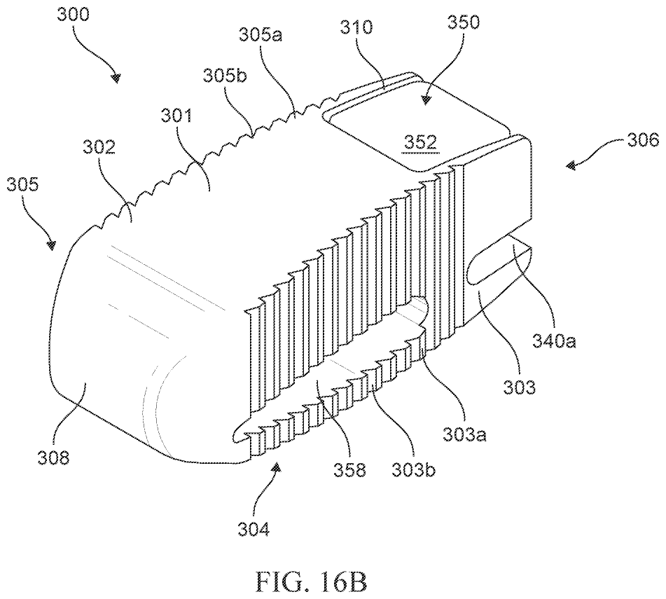

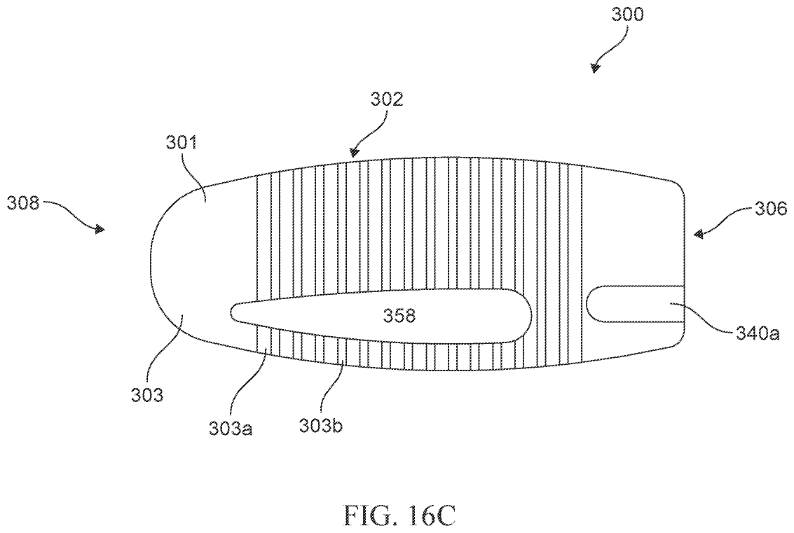

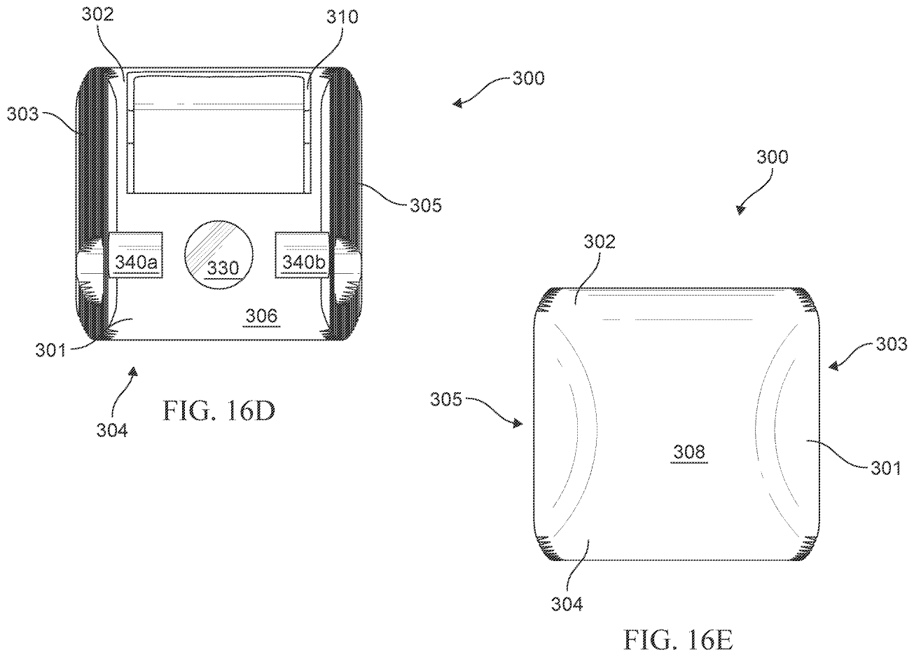

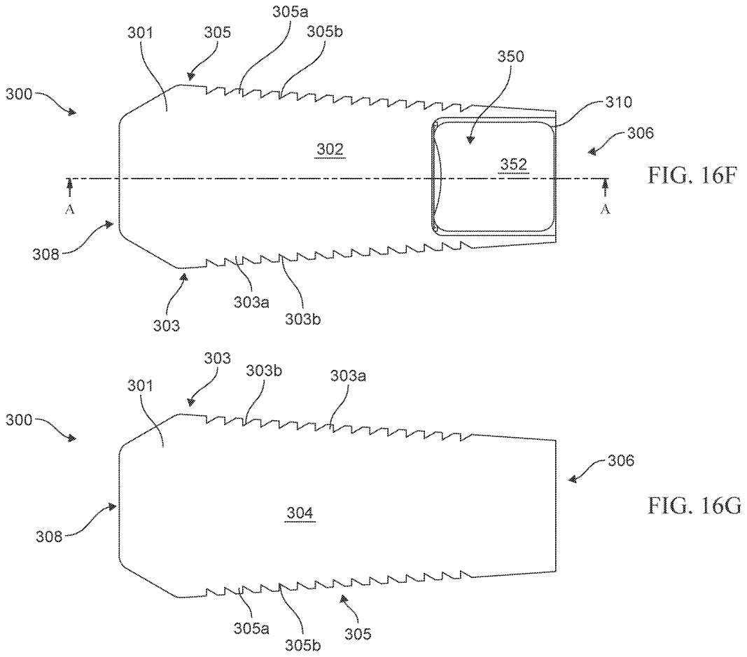

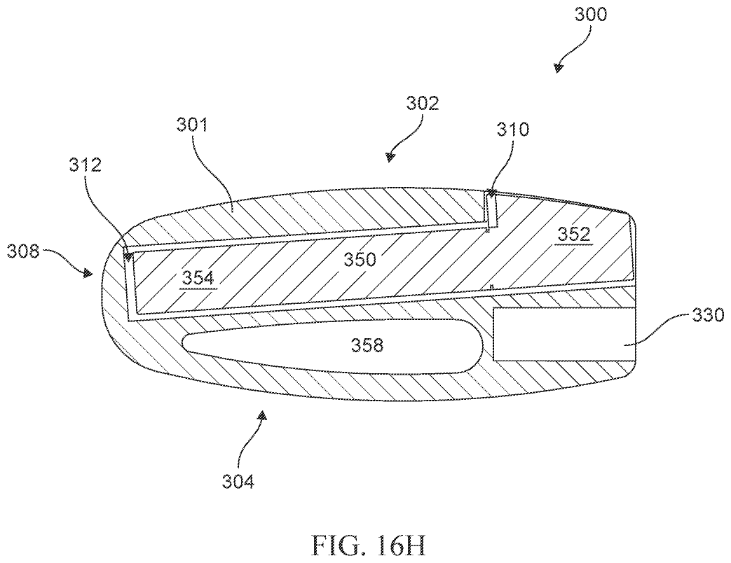

FIG. 18 illustrates a schematic of an implantable reporting processor cartridge for insertion into the interbody spacers illustrated in FIGS. 16A-161 and 17.

FIG. 19A illustrates an embodiment of a locking structure for retaining the cartridge of FIG. 18 within an interbody spacer.

FIG. 19B illustrates another embodiment of a locking structure for retaining the cartridge of FIG. 18 within an interbody spacer.

FIG. 19C illustrates another embodiment of a locking structure for retaining the cartridge of FIG. 18 within an interbody spacer.

FIG. 20A illustrates a cross-sectional view of a cartridge with an antenna positioned within an interbody spacer.

FIG. 20B illustrates a frontal view of the cartridge with an antenna positioned within the interbody spacer of FIG. 20A.

### DETAILED DESCRIPTION

As our population ages, there becomes an increasing need for devices such as spinal implants to alleviate pain and treat various conditions. Spinal implants can be used to treat deformity, stabilize and strengthen the spine, and to facilitate fusion. Once the spinal implant—devices such as interbody spacers and spinal cages-is inserted into the patient, the physician must determine whether the surgery has helped to address the patient's condition. A successful surgery should allow a patient to return to normal life with minimal pain. Traditionally, to evaluate the success of the spinal implant, the physician will rely on the patient indicating they are experiencing pain or having difficulty resuming activities as a measure of the patient's condition. However, relying on patient feedback can be limiting as a patient may not understand how to articulate the discomfort they are experiencing. As well, patients can be unreliable narrators of their physical activity. As well, there are limitations with relying solely on indirect measurements as the physician is unable to diagnose any problems associated with problems associated with the spinal procedure and the location of the spinal implant. For example, the interbody spacer or spinal cage can migrate (i.e., movement away from the point of implantation) and/or subside (i.e., the decrease in vertical height of the vertebral disc space prior to the completion of fusion) after surgery, causing pain for the patient.

In presently disclosed sensor assembly is configured to allow a physician to measure a patient's recovery both indirectly and/or directly. The sensor assembly can measure a patient's condition indirectly be monitoring a patient's general movement and lifestyle changes. For example, if the patient is unable to return to activities the patient previously enjoyed, it is an indication that perhaps the spinal implant did not address the patient's condition. As well, if the patient is experiencing pain, has an altered gait, or has greatly reduced his or her physical activity, this can also be an indirect measure that the patient is continuing to experience back pain and that the spinal implant has failed to address the patient's condition. As will be discussed in more detail below, the spinal implant includes a number of sensors that can allow a physician to determine whether a patient's general mobility has changed. This can include, for example, a change in how frequently a patient is walking, whether a patient is walking with a stoop, and whether a patient has a change in gait (e.g., limping). Combined with lifestyle information from the patient (e.g., feeling pain or continued participation in enjoyed activities), these changes can inform a patient that additional surgery may be necessary to address a patient's condition.

However, indirect measurements of a patient's condition can be limiting as it does not answer the “why” of a patient's pain. As will be discussed, the disclosed sensor assembly includes a number of sensors that are able to determine whether fusion between the vertebra have occurred, whether the spinal implant has migrated, and/or whether the spinal implant is experiencing subsidence. This additional information can provide the physician with the ability to more accurately and precisely diagnose a patient's condition and pain experienced.

Disclosed is an integrated sensor technology for spinal fusion procedures via an implantable interbody and/or posterior instrumentation device(s) which provide patients and clinicians clinically relevant objective data postsurgery (a subset of similar data may be captured preoperatively with a wearable device containing the same sensor(s)). A plurality of sensors within the implant device (or wearable) (accelerometers, gyroscopes, force gauge, strain gauge, temperature sensor, etc.) collect raw data passively or on-demand, which can be uploaded to a cloud infrastructure to be processed into clinical metrics, then made available for display on specified user interfaces.

In some embodiments, the processed data provide metrics as a tool for physicians to monitor their patients. Measured processed data from algorithms and methods directed to patient movement can yield but are not limited to: step count (number of steps/day), walking distance/day, average walking speed, spine angle, patient position (laying, hunching, or upright), and spine range of motion.

In some examples, the processed and analyzed data collected from the intelligent implant can provide patient outcomes monitoring such as percent fusion or earlier detection of complications (e.g., loosening/nonfusion, subsidence, migration) in an intelligent implant such as a spinal implant. A plurality of analyses and calculations can be used to determine these metrics. For example, for detecting percent fusion, vibration analysis is algorithmically performed utilizing in-vivo prosthesis vibration data from accelerometers (or other sensors). This vibration can be explicitly detected and correlated to established moments in the gait cycle (i.e., heel strike, mid-stance, toe off, etc.) or other established motions of mobility. By correlating vibration patterns to known movements, exact moments of interest can be analyzed. Analyzing spine fusion over time, the vibration amplitude would lessen as the bone grows and fuses adjacent vertebrae, therefore showing spinal fusion.

The present disclosure may be understood more readily by reference to the following detailed description of embodiments of the disclosure and the examples of implantable medical devices with implantable reporting processors. The following description, along with the accompanying drawings, sets forth certain specific details to provide a thorough understanding of various disclosed embodiments. However, one skilled in the relevant art will recognize that the disclosed embodiments may be practiced in various combinations, without one or more of these specific details, or with other methods, components, devices, materials, etc. In other instances, well-known structures or components that are associated with the environment of the present disclosure, including but not limited to the communication systems and networks, have not been shown or described to avoid unnecessarily obscuring descriptions of the embodiments.

The present disclosure refers to surgical procedures, for example, spinal procedures like spinal fusion which term includes reference to the surgery and associated implantable medical devices such as a spinal implant system (e.g., a spinal fusion implant such as a spinal interbody cage or spacer, rod or plate, or a spinal non-fusion implant such as an artificial disc or expandable rod).

Prior to setting forth this disclosure in more detail, it may be helpful to an understanding thereof to provide definitions of certain terms to be used herein. Additional definitions are set forth throughout this disclosure. The terms “include” and “comprise,” as well as derivatives thereof, mean inclusion without limitation. The term “or,” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like. The term “controller” means any device, system, or part thereof that controls at least one operation, such a device may be implemented in hardware (e.g., electronic circuitry), firmware, or software, or some combination of at least two of the same. The functionality associated with any particular controller may be centralized or distributed, whether locally or remotely. Other definitions of certain words and phrases may be provided within this patent document. Those of ordinary skill in the art will understand that in many, if not most instances, such definitions apply to prior as well as future uses of such defined words and phrases.

An “implantable medical device” as used in the present disclosure, is an implantable or implanted medical device that desirably replaces or functionally supplements a subject's natural body part. As used herein, the term “intelligent implant” refers to an implantable medical device with an implantable reporting processor. “Intelligent implant” is interchangeably referred to as “implantable sensor assembly” or “smart device.”

In some embodiment, the intelligent implant is an implanted or implantable medical device having an implantable reporting processor arranged to perform the functions as described herein. The intelligent implant may perform one or more of the following exemplary actions in order to characterize the post-implantation status of the intelligent implant: identifying the intelligent implant or a portion of the intelligent implant, e.g., by recognizing one or more unique identification codes for the intelligent implant or a portion of the intelligent implant; detecting, sensing and/or measuring parameters, which may collectively be referred to as monitoring parameters, in order to collect operational, kinematic, or other data about the intelligent implant or a portion of the intelligent implant and wherein such data may optionally be collected as a function of time; storing the collected data within the intelligent implant or a portion of the intelligent implant; and communicating the collected data and/or the stored data by a wireless means from the intelligent implant or a portion of the intelligent implant to an external computing device. The external computing device may have or otherwise have access to at least one data storage location such as found on a personal computer, a base station, a computer network, a cloud-based storage system, or another computing device that has access to such storage.

“Kinematic data,” as used herein, individually or collectively includes some or all data associated with a particular intelligent implant and available for communication outside of the particular intelligent implant. For example, kinematic data may include raw data from one or more sensors of an intelligent implant, wherein the one or more sensors include such as gyroscopes, accelerometers, pedometers, strain gauges, and the like that produce data associated with motion, force, tension, velocity, or other mechanical forces. Kinematic data may also include processed data from one or more sensors, status data, operational data, control data, fault data, time data, scheduled data, event data, log data, and the like associated with the particular intelligent implant. In some cases, high resolution kinematic data includes kinematic data from one, many, or all the sensors of the intelligent implant that is collected in higher quantities, resolution, from more sensors, more frequently, or the like.

In some embodiments, kinematics refers to the measurement of the positions, angles, velocities, and accelerations of body segments and joints during motion. Position describes the location of a body segment or joint in space, measured in terms of distance, e.g., in meters. A related measurement called displacement refers to the position with respect to a starting position. In two dimensions, the position is given in Cartesian co-ordinates, with horizontal followed by vertical position.

“Sensor” refers to a device that can be utilized to do one or more of detect, measure and/or monitor: 1) one or more different aspects of a body tissue (anatomy, physiology, metabolism, and/or function), 2) one or more aspects of body or body segment/joint condition or function (healing, motion including measurement of the positions, angles, velocities, and accelerations of body segments and joints), and/or 3) one or more aspects of the implant. Representative examples of sensors suitable for use within the present disclosure include, for example, fluid pressure sensors, fluid volume sensors, contact sensors, position sensors, pulse pressure sensors, blood volume sensors, blood flow sensors, chemistry sensors (e.g., for blood and/or other fluids), metabolic sensors (e.g., for blood and/or other fluids), accelerometers, mechanical stress sensors and temperature sensors. Within certain embodiments the sensor can be a wireless sensor, or, within other embodiments, a sensor connected to a wireless microprocessor. Within further embodiments one or more (including all) of the sensors can have a Unique Sensor Identification number (“USI”) which specifically identifies the sensor. In certain embodiments, the sensor is a device that can be utilized to measure in a quantitative manner, one or more different aspects of a body tissue (anatomy, physiology, metabolism, and/or function) and/or one or more aspects of the implant. In certain embodiments, the sensor is an accelerometer that can be utilized to measure in a quantitative manner, one or more different aspects of a body tissue (e.g., function) and/or one or more aspects of the implant (e.g., alignment in the patient).

A wide variety of sensors (also referred to as Microelectromechanical Systems or “MEMS,” or Nanoelectromechanical Systems or “NEMS,” and BioMEMS or BioNEMS) can be utilized within the present disclosure.

### Intelligent Implant Systems

An intelligent implant system includes one or more of 1) a sensor that detects and/or measures the functioning of the implant and/or the immediate environment around the implant and/or the activity of the patient, 2) memory that stores data from that detection and/or measuring, 3) an antenna that transmits that data; 4) a base station that receives the data generated by the sensor and may transmit the data and/or analyzed data to a cloud-based location; 5) a cloud-based location where data may be stored and analyzed, and analyzed data may be stored and/or further analyzed; 6) a receiving terminal that receives output from the cloud-based location, where that receiving terminal may be accessed, e.g., by a health care professional or an insurance company or the manufacturer of the implant, and the output may identify the status of the implant and/or the functioning of the implant and/or the status of the patient who has received the implant, and may also provide recommendations for addressing any concerns raised by analysis of the original data.

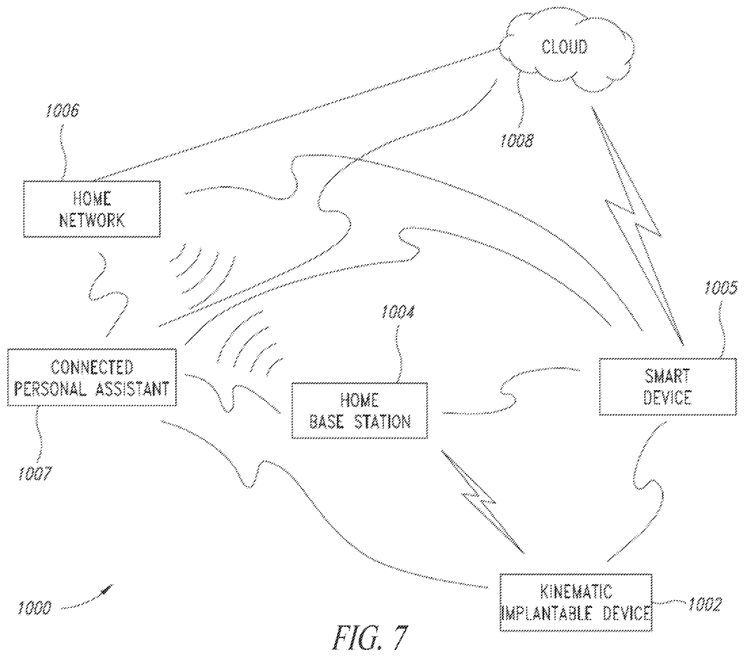

FIG. 7 illustrates a context diagram of an intelligent implant environment 1000. It should be recognized that no individual component of the implant environment 1000 is essential or indispensable. Any component described herein may be practiced using any device suitable for performing the recited functions.

In the environment, an intelligent implant 1002 is implanted by a medical practitioner (not shown in FIG. 7) in the body of a patient (not shown in FIG. 7). The intelligent implant 1002 has an associated (IRP) that is arranged and configured to collect data including, for example, medical and health data related to a patient to which the device is associated, operational data of the device 1002 along with kinematic data associated with particular movement of the patient or particular movement of a portion of the patient's body, and operational data of the device 1002 itself. The intelligent implant 1002 communicates with one or more base stations 1004 or one or more smart devices 1005 during different stages of monitoring the patient.

For example, in association with a medical procedure, an intelligent implant 1002 is implanted in the patient's body. Coetaneous with the medical procedure, the intelligent implant 1002 communicates with an operating room base station (not shown in FIG. 7). Subsequently, after sufficient recovery from the medical procedure, the patient returns home wherein the intelligent implant 1002 is arranged to communicate with a home base station 1004. It is understood that the home base station may be a separate device or it could be a smart phone with a customized application containing software enabling communication with the smart implant. At other times, the intelligent implant 1002 is arranged to communicate with a doctor office base station (not shown in FIG. 7). The intelligent implant 1002 communicates with each base station 1004 via a short range network protocol, such as the medical implant communication service (MICS), the medical device radio communications service (MedRadio), or some other wireless communication protocol suitable for use with the intelligent implant 1002.

The intelligent implant 1002 includes one or more measurement units to collect information and data associated with the use of the body part to which the intelligent implant 1002 is associated. In some embodiments, the information and data collected is medical and health data related to a patient to which the device is associated. For example, the intelligent implant 1002 may include an inertial measurement unit that includes gyroscope(s), accelerometer(s), pedometer(s), or other kinematic sensors to collect acceleration data for the medial/lateral, anterior/posterior, and anterior/inferior axes of the associated body part; angular velocity for the sagittal, frontal, and transvers planes of the associated body part; force, stress, tension, pressure, duress, migration, vibration, flexure, rigidity, or some other measurable data.

The intelligent implant 1002 collects data at various times and at various rates during a monitoring process of the patient. In some embodiments, the intelligent implant 1002 may operate in a plurality of different phases over the course of monitoring the patient so that more data is collected soon after the intelligent implant 1002 is implanted into the patient, but less data is collected as the patient heals and thereafter.

The amount and type of data collected by an intelligent implant 1002 may be different from patient to patient, and the amount and type of data collected may change for a single patient. For example, a medical practitioner studying data collected by the intelligent implant 1002 of a particular patient may adjust or otherwise control how the intelligent implant 1002 collects future data.

The amount and type of data collected by an intelligent implant 1002 may be different for different body parts, for different types of movement, for different types of patient conditions, for different patient demographics, or for other differences. Alternatively, or in addition, the amount and type of data collected may change overtime based on other factors, such as how the patient is healing or feeling, how long the monitoring process is projected to last, how much battery power remains and should be conserved, the type of movement being monitored, the body part being monitored, and the like. In some cases, the collected data is supplemented with personally descriptive information provided by the patient such as subjective pain data, quality of life metric data, co-morbidities, perceptions, or expectations that the patient associates with the intelligent implant 1002, or the like.

In some embodiments, the intelligent implant 1002 is implanted into a patient to monitor movement or other aspects of a particular body part or the intelligent implant 1002 itself. Implantation of the intelligent implant 1002 into the patient may occur in an operating room. As used herein, operating room includes any office, room, building, or facility where the intelligent implant 1002 is implanted into the patient. For example, the operating room may be a typical operating room in a hospital, an operating room in a surgical clinic or a doctor's office, or any other operating theater where the intelligent implant 1002 is implanted into the patient.

The operating room base station (not shown in FIG. 7) is utilized to configure and initialize the intelligent implant 1002 in association with the intelligent implant 1002 being implanted into the patient. A communicative relationship is formed between the intelligent implant 1002 and the operating room base station, for example, based on a polling signal transmitted by the operating room base station and a response signal transmitted by the intelligent implant 1002.

Upon forming a communicative relationship, which will often occur prior to implantation of the intelligent implant 1002, the operating room base station (not shown in FIG. 7) transmits initial configuration information to the intelligent implant 1002. This initial configuration information may include, but is not limited to, a time stamp, a day stamp, an identification of the type and placement of the intelligent implant 1002, information on other implants associated with the intelligent implant, surgeon information, patient identification, operating room information, and the like.

In some embodiments, the initial configuration information is passed unidirectionally; in other embodiments, initial configuration is passed bidirectionally. The initial configuration information may define at least one parameter associated with the collection of data by the intelligent implant 1002. For example, the configuration information may identify settings for one or more sensors on the intelligent implant 1002 (e.g., accelerometer range, accelerometer output data rate, gyroscope range, gyroscope output data rate, and the like) for each of one or more modes of operation. The configuration information may also include other control information, such as an initial mode of operation of the intelligent implant 1002, a particular event that triggers a change in the mode of operation, radio settings, data collection information (e.g., how often the intelligent implant 1002 wakes up to collected data, how long it collects data, how much data to collect), home base station 1004, smart device 1005, and connected personal assistant 1007 identification information, and other control information associated with the implantation or operation of the intelligent implant 1002. Examples of the connected personal assistant 1007, which also can be called a smart speaker, include Amazon Echo®, Amazon Dot®, Google Home®, Philips® patient monitor, Comcast's health-tracking speaker, and Apple HomePod®.

In some embodiments, the configuration information may be pre-stored on the operating room base station (not shown in FIG. 7) or an associated computing device. In other embodiments, a surgeon, surgical technician, or some other medical practitioner may input the control information and other parameters to the operating room base station for transmission to the intelligent implant 1002. In some embodiments, the operating room base station may communicate with an operating room configuration computing device (not shown in FIG. 7). The operating room configuration computing device includes an application with a graphical user interface that enables the medical practitioner to input configuration information for the intelligent implant 1002. In various embodiments, the application executing on the operating room configuration computing device may have some of the configuration information predefined, which may or may not be adjustable by the medical practitioner.

The operating room configuration computing device (not shown in FIG. 7) communicates the configuration information to the operating room base station (not shown in FIG. 7) via a wired or wireless network connection (e.g., via a USB connection, Bluetooth connection, Bluetooth Low Energy (BTLE) connection, or Wi-Fi connection), which in turn communicates it to the intelligent implant 1002.

The operating room configuration computing device (not shown in FIG. 7) may also display information regarding the intelligent implant 1002 or the operating room base station (not shown in FIG. 7) to the surgeon, surgical technician, or other medical practitioner. For example, the operating room configuration computing device may display error information if the intelligent implant 1002 is unable to store or access the configuration information, if the intelligent implant 1002 is unresponsive, if the intelligent implant 1002 identifies an issue with one of the sensors or radio during an initial self-test, if the operating room base station (not shown in FIG. 7) is unresponsive or malfunctions, or for other reasons.

Although the operating room base station (not shown in FIG. 7) and the operating room configuration computing device (not shown in FIG. 7) are described as separate devices, embodiments are not so limited; rather, the functionality of the operating room configuration computing device and the operating room base station may be included in a single computing device or in separate devices as illustrated. In this way, the medical practitioner may be enabled in one embodiment to input the configuration information directly into the operating room base station.

Returning to FIG. 7, once the intelligent implant 1002 is implanted into the patient and the patient returns home, the home base station 1004, the smart device 1005 (e.g., the patient's smart phone), the connected personal assistant 1007, or two or more of the home base station, and the smart device, and the connected personal assistant can communicate with the intelligent implant 1002. The intelligent implant 1002 can collect data at determined rates and times, variable rates and times, or otherwise controllable rates and times. Data collection can start when the intelligent implant 1002 is initialized in the operating room, when directed by a medical practitioner, or at some later point in time. At least some data collected by the intelligent implant 1002 may be transmitted to the home base station 1004 directly, to the smart device 1005 directly, to the connected personal assistant 1007 directly, to the base station via one or both of the smart device and the connected personal assistant, to the smart device via one or both of the base station and the connected personal assistant, or to the connected personal assistant via one or both of the smart device and the base station. Here, “one or both” means via an item alone, and via both items serially or in parallel. For example, data collected by the intelligent implant 1002 may be transmitted to the home base station 1004 via the smart device 1005 alone, via the connected personal assistant 1007 alone, serially via the smart device and the connected personal assistant, serially via the connected personal assistant and the smart device, and directly, and possibly contemporaneously, via both the smart device and the connected personal assistant. Similarly, data collected by the intelligent implant 1002 may be transmitted to the smart device 1005 via the home base station 1004 alone, via the connected personal assistant 1007 alone, serially via the home base station and the connected personal assistant, serially via the connected personal assistant and the home base station, and directly, and possibly contemporaneously, via both the home base station and the connected personal assistant. Further in example, data collected by the intelligent implant 1002 may be transmitted to the connected personal assistant 1007 via the smart device 1005 alone, via the home base station 1004 alone, serially via the smart device and the home base station, serially via the home base station and the smart device, and directly, and possibly contemporaneously, via both the smart device and the home base station.

In various embodiments, one or more of the home base station 1004, the smart device 1005, and the connected personal assistant 1007 pings the intelligent implant 1002 at periodic, predetermined, or other times to determine if the intelligent implant 1002 is within communication range of one or more of the home base station, the smart device, and the connected personal assistant. Based on a response from the intelligent implant 1002, one or more of the home base station 1004, the smart device 1005, and the connected personal assistant 1007 determines that the intelligent implant 1002 is within communication range, and the intelligent implant 1002 can be requested, commanded, or otherwise directed to transmit the data it has collected to one or more of the home base station 1004, the smart device 1005, and the connected personal assistant 1007.

Each of one or more of the home base station 1004, the smart device 1005, and the connected personal assistant 1007 may, in some cases, be arranged with a respective optional user interface. The user interface may be formed as a multimedia interface that unidirectionally or bidirectionally passes one or more types of multimedia information (e.g., video, audio, tactile, etc.). Via the respective user interface of one or more of the home base station 1004, the smart device 1005, and the connected personal assistant 1007, the patient (not shown in FIG. 7) or an associate (not shown in FIG. 7) of the patient may enter other data to supplement the data collected by the intelligent implant 1002. A user, for example, may enter personally descriptive information (e.g., age change, weight change), changes in medical condition, co-morbidities, pain levels, quality of life, an indication of how the implantable device 1002 “feels,” or other subjective metric data, personal messages for a medical practitioner, and the like. In these embodiments, the personally descriptive information may be entered with a keyboard, mouse, touch-screen, microphone, wired or wireless computing interface, or some other input means. In cases where the personally descriptive information is collected, the personally descriptive information may include, or otherwise be associated with, one or more identifiers that associate the information with unique identifier of the intelligent implant 1002, the patient, an associated medical practitioner, an associated medical facility, or the like.

In some of these cases, a respective optional user interface of each of one or more of the home base station 1004, the smart device 1005, and the connected personal assistant 1007 may also be arranged to deliver information associated with the intelligent implant 1002 to the user from, for example, a medical practitioner. In these cases, the information delivered to the user may be delivered via a video screen, an audio output device, a tactile transducer, a wired or wireless computing interface, etc.

In some embodiments, where one or more of the home base station 1004, the smart device 1005, and the connected personal assistant 1007 are arranged with a user interface, which may be formed with an internal user interface arranged for communicative coupling to a patient portal device. The patient portal device may be smartphone, a tablet, a body-worn device, a weight or other health measurement device (e.g., thermometer, bathroom scale, etc.), or some other computing device capable of wired or wireless communication. In these embodiments, the user can enter the personally descriptive information, and the user also may be able to receive information associated with the implantable device 1002.

The home base station 1004 utilizes a home network 1006 of the patient to transmit the collected data to cloud 1008. The home network 1006, which may be a local area network, provides access from the home of the patient to a wide area network, such as the internet. In some embodiments, the home base station 1004 may utilize a Wi-Fi connection to connect to the home network 1006 and access the internet. In other embodiments, the home base station 1004 may be connected to a home computer (not shown in FIG. 7) of the patient, such as via a USB connection, which itself is connected to the home network 1006.

The smart device 1005 can communicate with the intelligent implant 1002 directly via, for example, Bluetooth® compatible signals, and can utilize the home network 1006 of the patient to transmit the collected data to cloud 1008, or can communicate directly with the cloud, for example, via a cellular network. Alternatively, the smart device 1005 can be configured to communicate directly with one or both of the base station 1004 and the connected personal assistant 1007 via, for example, Bluetooth® compatible signals, and rather than communicate directly with the intelligent implant 1002.

Furthermore, the connected personal assistant 1007 can communicate with the intelligent implant 1002 directly via, for example, Bluetooth® compatible signals, and can utilize the home network 1006 of the patient to transmit the collected data to cloud 1008, or can communicate directly with the cloud, for example, via a modem/internet connection or a cellular network. Alternatively, the connected personal assistant 1007 is configured to communicate directly with one or both of the base station 1004 and the smart device 1005 via, for example, Bluetooth® compatible signals, and is not configured to communicate directly with the intelligent implant 1002.

Along with transmitting collected data to the cloud 1008, one or more of the home base station 1004, the smart device 1005, and the connected personal assistant 1007 may also obtain data, commands, or other information from the cloud 1008 directly or via the home network 1006. One or more of the home base station 1004, the smart device 1005, and the connected personal assistant 1007 may provide some or all the received data, commands, or other information to the intelligent implant 1002. Examples of such information include, but are not limited to, updated configuration information, diagnostic requests to determine if the intelligent implant 1002 is functioning properly, data collection requests, and other information.

The cloud 1008 can include one or more server computers or databases to aggregate data collected from the intelligent implant 1002, and in some cases personally descriptive information collected from a patient (not shown in FIG. 7), with data collected from other intelligent implants (not illustrated), and in some cases personally descriptive information collected from other patients. In this way, the cloud 1008 can create a variety of different metrics regarding collected data from each of a plurality of intelligent implants that are implanted into separate patients. This information can be helpful in determining if the intelligent implants are functioning properly. The collected information may also be helpful for other purposes, such as determining which specific devices may not be functioning properly, determining if a procedure or condition associated with the intelligent implant is helping the patient, and determining other medical information.

At various times throughout the monitoring process, the medical practitioner may request the patient for follow up appointments. This medical practitioner may be the surgeon who implanted the intelligent implant 1002 in the patient or a different medical practitioner that supervises the monitoring process, physical therapy, and recovery of the patient. For a variety of different reasons, the medical practitioner may want to collect real-time data from the intelligent implant 1002 in a controlled environment. In some cases, the request to visit the medical practitioner may be delivered through a respective optional bidirectional user interface of each of one or more of the home base station 1004, the smart device 1005, and the connected personal assistant 1007.

A medical practitioner utilizes the doctor office base station (not shown in FIG. 7), which communicates with the intelligent implant 1002, to pass additional data between the doctor office base station and the intelligent implant 1002. In some embodiments, the medical practitioner utilizes the doctor office base station (not shown in FIG. 7) to pass commands to the intelligent implant 1002. In some embodiments, the doctor office base station instructs the intelligent implant 1002 to enter a high-resolution mode to temporarily increase the rate or type of data that is collected for a short time. The high-resolution mode directs the intelligent implant 1002 to collect different (e.g., large) amounts of data during an activity where the medical practitioner is also monitoring the patient.

In some embodiments, the doctor office base station (not shown in FIG. 7) enables the medical practitioner to input event or pain markers, which can be synchronized with the high-resolution data collected by the intelligent implant 1002. For example, assume the intelligent implant 1002 is a component positioned in a spine. The medical practitioner can have the patient walk on a treadmill while the intelligent implant 1002 is in the high-resolution mode. As the patient walks, the patient may complain about pain in his/her back. The medical practitioner can click a pain marker button on the doctor office base station to indicate the patient's discomfort. The doctor office base station records the marker and the time at which the marker was input. When the timing of this marker is synchronized with the timing of the collected high-resolution data, the medical practitioner can analyze the data to try and determine the cause of the pain.

In other embodiments, the doctor office base station (not shown in FIG. 7) may provide updated configuration information to the intelligent implant 1002. The intelligent implant 1002 can store this updated configuration information, which can be used to adjust the parameters associated with the collection of the data. For example, if the patient is doing well, the medical practitioner can direct a reduction in the frequency at which the intelligent implant 1002 collects data. On the contrary, if the patient is experiencing an unexpected amount of pain, the medical practitioner may direct the intelligent implant 1002 to collect additional data for a determined period of time (e.g., a few days). The medical practitioner may use the additional data to diagnose and treat a particular problem. In some cases, the additional data may include personally descriptive information provided by the patient (not shown in FIG. 7) after the patient has left presence of the medical practitioner and is no longer in range of the doctor office base station. In these cases, the personally descriptive information may be collected and delivered from via one or more of the home base station 1004, the smart device 1005, and the connected personal assistant 1007. Firmware within the intelligent implant and/or the base station will provide safeguards limiting the duration of such enhanced monitoring to ensure the battery retains sufficient power to last for the implant's lifecycle.

In some embodiments, the doctor office base station (not shown in FIG. 7) may communicate with a doctor office configuration computing device (not shown in FIG. 7). The doctor office configuration computing device includes an application with a graphical user interface that enables the medical practitioner to input commands and data. Some or all the commands, data, and other information may be later transmitted to the intelligent implant 1002 via the doctor office base station. For example, in some embodiments, the medical practitioner can use the graphical user interface to instruct the intelligent implant 1002 to enter its high-resolution mode. In other embodiments, the medical practitioner can use graphical user interface to input or modify the configuration information for the intelligent implant 1002. The doctor office configuration computing device transmits the information (e.g., commands, data, or other information) to the doctor office base station via a wired or wireless network connection (e.g., via a USB connection, Bluetooth connection, or Wi-Fi connection), which in turn transmits some or all the information to the intelligent implant 1002.

The doctor office configuration computing device (not shown in FIG. 7) may also display, to the medical practitioner, other information regarding the intelligent implant 1002, regarding the patient (e.g., personally descriptive information), or the doctor office base station. For example, the doctor office configuration computing device may display the high-resolution data that is collected by the intelligent implant 1002 and transmitted to the doctor office base station (not shown in FIG. 7). The doctor office configuration computing device may also display error information if the intelligent implant 1002 is unable to store or access the configuration information, if the intelligent implant 1002 is unresponsive, if the intelligent implant 1002 identifies an issue with one of the sensors or radio, if the doctor office base station is unresponsive or malfunctions, or for other reasons.

In some embodiments, doctor office configuration computing device (not shown in FIG. 7) may have access to the cloud 1008. In at least one embodiment, the medical practitioner can utilize the doctor office configuration computing device to access data stored in the cloud 1008, which was previously collected by the intelligent implant 1002 and transmitted to the cloud 1008 via one or both of the home base station 1004 and smart device 1005. Similarly, the doctor office configuration computing device can transmit the high-resolution data obtain from the intelligent implant 1002 via the doctor office base station to the cloud 1008. In some embodiments, the doctor office base station may have internet access and may be enabled to transmit the high-resolution data directly to the cloud 1008 without the use of the doctor office configuration computing device.

In some embodiments, the medical practitioner may update the configuration information of the intelligent implant 1002 when the patient is not in the medical practitioner's office. In these cases, the medical practitioner can utilize the doctor office configuration computing device (not shown in FIG. 7) to transmit updated configuration information to the intelligent implant 1002 via the cloud 1008. One or more of the home base station 1004, the smart device 1005, and the connected personal assistant 1007 can obtain updated configuration information from the cloud 1008 and pass updated configuration information to the cloud. This can allow the medical practitioner to remotely adjust the operation of the intelligent implant 1002 without needing the patient to come to the medical practitioner's office. This may also permit the medical practitioner to send messages to the patient (not shown in FIG. 7) in response, for example, to personally descriptive information that was provided by the patient and passed through one or more of the home base station 1004, the smart device 1005, and the connected personal assistant 1007 to the doctor office base station (not shown in FIG. 7). For example, if a patient having undergone spinal fusion speaks “my back hurts when I walk” into the connected personal assistant 1007, then the medical practitioner may issue a prescription for a pain reliever and cause the connected personal assistant to notify the patient by “speaking” “the doctor has called in a prescription for Vicodin® to your preferred pharmacy; the prescription will be ready for pick up at 4pm.”

Although the doctor office base station (not shown in FIG. 7) and the doctor office configuration computing device (not shown in FIG. 7) are described as separate devices, embodiments are not so limited; rather, the functionality of the doctor office configuration computing device and the doctor office base station may be included in a single computing device or in separate devices (as illustrated). In this way, the medical practitioner may be enabled in one embodiment to input the configuration information or markers directly into the doctor office base station and view the high-resolution data (and synchronized marker information) from a display on the doctor office base station.

Still referring to FIG. 7, alternate embodiments are contemplated. For example, one or two of the home base station 1004, the smart device 1005, and the connected personal assistant 1007 may be omitted from the intelligent implant environment 1000. Furthermore, each of the base station 1004, the smart device 1005, and the connected personal assistant 1007 may be configured to communicate with one or both of the implantable device 1002 and the cloud 1008 via another one or two of the base station, the smart device, and the connected personal assistant. Moreover, the smart device 1005 can be temporarily contracted as an interface to the implantable device 1002 and can be any suitable device other than a smart phone, such as a smart watch, a smart patch, and any Internet of Things (IoT) device, such as a coffee pot, capable of acting as an interface to the implantable device 1002. In addition, one or more of the base station 1004, smart device 1005, and connected personal assistant 1007 can act as a communication hub for multiple prostheses implanted in one or more patients. Furthermore, one or more of the base station 1004, smart device 1005, and connected personal assistant 1007 can automatically order or reorder prescriptions or medical supplies in response to patient input or implantable-prosthesis input (e.g., pain level, instability level) if a medical professional and insurance company have preauthorized such an order or reorder; alternatively, one or more of the base station, smart device, and connected personal assistant can be configured to request, from a medical professional or an insurance company, authorization to place the order or reorder. Moreover, one or more of the base station 1004, smart device 1005, and connected personal assistant 1007 can be configured with a personal assistant such as Alexa® or Siri®.

### Intelligent Implant Systems

The present disclosure provides medical devices, including prostheses or medical devices which may be implanted into a patient (implants), which may be utilized to monitor and report the status and/or activities of the medical device, including post-surgical activities and progress of the patient involved, as well as features thereof. The present disclosure provides an intelligent implant or implantable sensor assembly that achieves the benefit of a medical implant, e.g., the benefit afforded by a prosthesis which replaces or supplements a natural function of a patient, while also achieving the benefit of monitoring and reporting, which provides insight into the function and/or condition of the device and/or the patient who has received the implanted device. The medical device can be an implantable device that is an in vivo implantable prosthesis that can be implanted into the body of a living host (also referred to as a patient), for example, to improve the function of, or to replace, a biological structure of the patient's body.

The present disclosure provides intelligent implants, e.g., an implantable medical device with an implantable reporting processor (IRP), also referred to herein as a “cartridge.” When the intelligent implant is included in a component of an implant system, the intelligent implant can monitor displacement or movement of the component or implant system. The intelligent implant can also provide kinematic data that can be used to assess the mobility and health of the patient in which the system is implanted.

Non-limiting and non-exhaustive list of embodiments of intelligent implants include components of a spinal implant coupled to a sensor. Examples of spinal devices and implants include pedicle screws, spinal rods, spinal wires, spinal plates, interbody spacers, spinal cages, artificial discs, facet implants, bone cement, as well as combinations of these (e.g., one or more pedicle screws and spinal rods, one or more pedicle screws and a spinal plate). In addition, medical delivery devices for the placement of spinal devices and implants, along with one or more sensors, may also be an intelligent medical device according to the present disclosure. Examples of medical delivery devices for spinal implants include kyphoplasty balloons, catheters (including thermal catheters and bone tunnel catheters), bone cement injection devices, microdiscectomy tools and other surgical tools.

In some embodiments, spinal devices and implants can be embodied by any type of interbody fusion device (Posterior Lateral Interbody Fusion (PLIF), a Transforaminal LIF (TLIF), an Anterior LIF (ALIF), or Lateral LIF (LLIF)) either in a single level or multiple level fusion (i.e., L4-L5 single level vs L1-L5 multi-level). These implants can be made of any biocompatible materials but are typically PEEK or Titanium.

In some embodiments an intelligent implant can comprise an implantable reporting processor (IRP) incorporated into a spinal cage or an interbody spacer used during spinal fusion. The interbody spacer can be inserted along any point of the patient's spine. For example, the interbody spacer can be inserted to provide for spinal fusion in the lumbar spine, thoracic spine, and/or cervical spine. The interbody spacer is configured to take the load from adjacent vertebrae and can include an opening that can receive an (IRP). In examples where a interbody spacer and separate IRP cartridge is used, the physician will couple the IRP with the interbody spacer to form the intelligent implant. As discussed elsewhere herein, in alternative configurations, the IRP may be integrated with the implant and contain any of the features of the cartridge IRP described herein. The physician can then fill the interbody spacer with material to help retain the IRP and provide for enhanced fusion between the adjacent vertebrae. For example, the interbody spacer can be filled with body material (e.g., bloody sawdust), other biological material, or other synthetics. As will be discussed in more detail below, the IRP can form a cartridge that can be permanently or reversibly insertable into the interbody spacer. The cartridge can include an antenna, an inertial measurement unit (IMU), and/or additional sensors that can sense and track a patient's movements.

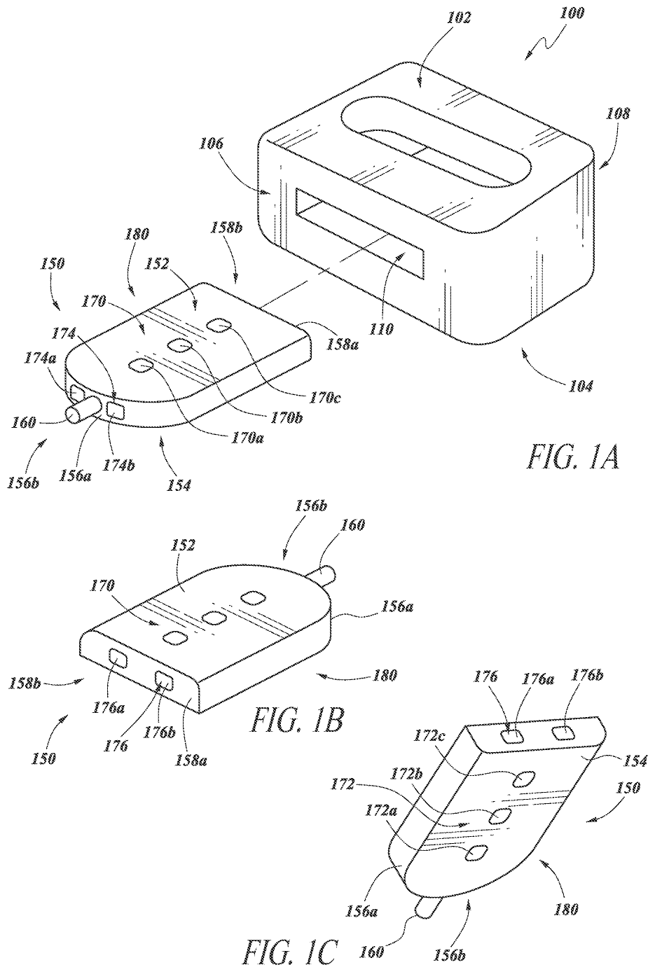

FIG. 1A illustrates a perspective view of an intelligent implant 100 in the form of an interbody spacer that includes an implantable reporting processor 150. The implantable reporting processor 150 can interface with a spinal cage/interbody spacer like that shown in FIG. 1A. For example, the components can be assembled intraoperatively. The interbody spacer can come in a variety of different shapes and sizes. As illustrated, the interbody spacer can comprise a top surface 102, a bottom surface 104, a medial surface 106, and lateral surface 108. The medial surface 106 can include an opening 110 and/or the lateral surface 108 can include an opening 112. The opening 110 and/or the opening 112 can allow the implantable reporting processor 150 to be inserted and secured within the interbody spacer of the intelligent implant 100.

The implantable reporting processor 150 can include a housing 180 that encloses a battery, an electronics assembly, an antenna, and/or one or more sensors. The one or more sensors can include any of the sensors described herein. The housing 180 can include a cover or a casing that encases and secures the various components of the implantable reporting processor 150. For example, as illustrated in FIG. 1A, the implantable reporting processor 150 is in the form of a cartridge for insertion into the patient. The cartridge can be any shape or size such that it can be inserted into the interbody spacer. For example, the implantable reporting processor 150 of FIG. 1A is thin and wafer shaped to allow it to be inserted through the opening 110 or the opening 112 of the interbody spacer. The implantable reportion processor 150 can be generally D-shaped with a generally curved medial end 156b and a generally straight lateral end 158b. However, the implantable reporting processor 150 can be cylindrical or any other shape or size. As shown in FIGS. 1B-1C, the implantable reporting processor 150 can include a housing 180 that covers and encloses the battery and the electronics assembly. In some embodiments, the implantable reporting processor 150 can include a top surface 152, a bottom surface 154, a medial surface 156a, and a lateral surface 158a. In some embodiments, the antenna 160 of the implantable reporting processor 150 can have a radome extending from the exterior profile of the implantable reporting processor 150, for example the medial end 156b of the implantable reporting processor 150. When assembled with the spinal cage/interbody spacer, the radome can protrude from the exterior profile of the spacer. This arrangement allows for communication even if the implantable reporting processor 150 and/or spacer has a metallic material. As described in more detail here, the implantable reporting processor 150 and/or spinal cage/interbody spacer can communicate with external devices, for example using Bluetooth Low Energy, to transmit collected data or receive programming and configuration data. The antenna 160 can be optimized for frequencies in the range of 2.4 to 2.5 GHz.

As will be discussed in more detail below, the implantable reporting processor 150 can include one or more sensors positioned on the various surfaces of the implantable reporting processor 150. As shown in FIGS. 1A-1C, the implantable reporting processor 150 can include at least one sensor 170. The at least one sensor 170 can be hermetically sealed and mounted inside or attached to a surface of the implantable reporting processor 150, for example on the top surface 152 of the implantable reporting processor 150. As illustrated in FIGS. 1A-1C, at least one sensor 170 of the implantable reporting processor 150 can include a sensor 170a, sensor 170b, and sensor 170c. The sensors 170a, 170b, 170c can be positioned in series.

The at least one sensor 170 can include strain or force sensors to detect strain or force on the surface of the implantable reporting processor 150 as means to detect fusion between two adjacent vertebrae in which the spacer is positioned because load increases as fusion progresses.

The at least one sensor 170 can include a vibration sensor detect acoustic emissions associated with scratching/abrasion of the interbody spacer against adjacent vertebrae. The extent of acoustic emissions changes (most likely decreasing) as intervertebral fusion progresses.

The vibration sensor can be combined with accelerometer(s) to collect acoustic emission (vibration) measurements when the patient is in known activities such as walking. The accelerometer can be low power AC or DC accelerometer. The accelerometer(s) can be hermetically sealed within the implantable reporting processor 150. The accelerometer(s) can measure the inclination angle of the spine relative to gravity vector; spine angle relative to the gravity vector provides a center of mass measurement; the center of mass measurement correlates to the recovery, pain level, and/or health status of the patient. The accelerometer(s) can measure activity patterns of the patient (such as walking) and trigger collection of data by the accelerometer(s) to occur during targeted activities such as walking.

Although the at least one sensor 170 is illustrated on the top surface of the implantable reporting processor 150, sensor(s) may be positioned on any other surface of the implantable reporting processor 150 as described below. Moreover, in other embodiments, the at least one sensor 170 may be incorporated into the spacer itself.

Additionally or alternatively, the implantable reporting processor 150 can include at least one sensor 172 on the bottom surface 154 of the implantable reporting processor 150. As illustrated in FIGS. 1A-1C, at least one sensor 172 of the implantable reporting processor 150 can include a sensor 172a, sensor 172b, and sensor 172c. The sensors 172a, 172b, 172c can be positioned in series.

In some configurations, the implantable reporting processor 150 can include at least one sensor 174 on the medial surface 156a of the implantable reporting processor 150. As illustrated in FIGS. 1A-1C, at least one sensor 174 of the implantable reporting processor 150 comprises a sensor 174a and a sensor 174b. However, at least one sensor 174 can include a greater or fewer number of sensors in various configurations. In some configurations, the implantable reporting processor 150 can include at least one sensor 176 on the lateral surface 158a of the implantable reporting processor 150. As illustrated in FIGS. 1A-1C, at least one sensor 176 of the implantable reporting processor 150 can comprises a sensor 176a and a sensor 176b. However, at least one sensor 176 can include a greater or fewer number of sensors in various configurations. In some embodiments, at least one sensor 170, at least one sensor 172, at least one sensor 174, and at least one sensor 176 can be embedded within the material of the housing 180.

The implantable reporting processor 150 can be in the form of a cartridge that can be inserted into the interbody spacer through either the opening 110 of the medial surface 106 or the opening 112 of the lateral surface 108. In some embodiments, the implantable reporting processor 150 can form a positive connection with the interbody spacer before the interbody spacer is inserted into the patient. For example, the positive connection can be any of a number of mechanical connections such as snap fits, locks, twists, mated threads, etc. In some embodiments, the implantable reporting processor 150 can be inserted into the interbody spacer reversibly. The reversible connection between the implantable reporting processor 150 and the interbody spacer can provide the physician with the option of removing the cartridge in situations where any maintenance may need to be performed on the implantable reporting processor 150 once it is inserted into the patient. A reversible connection can allow the implantable reporting processor 150 to be removed in the instance when a battery may need to be replaced.

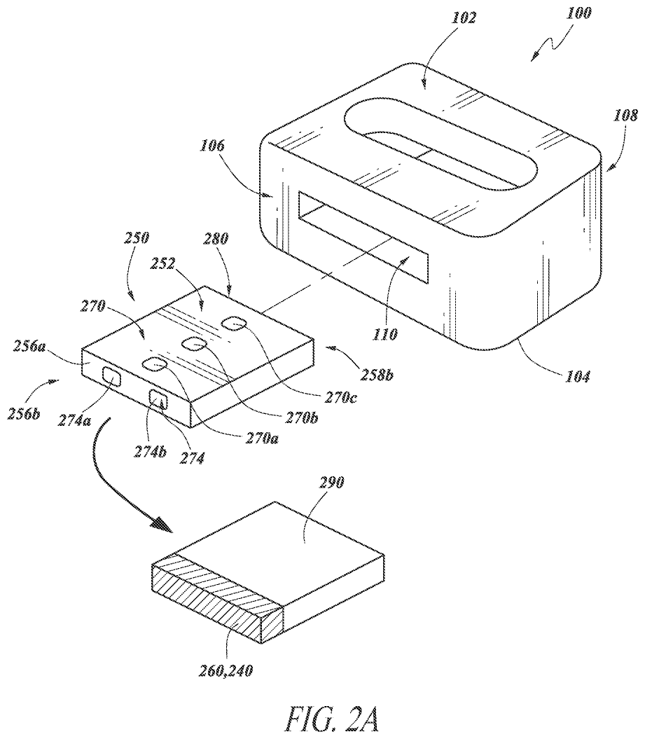

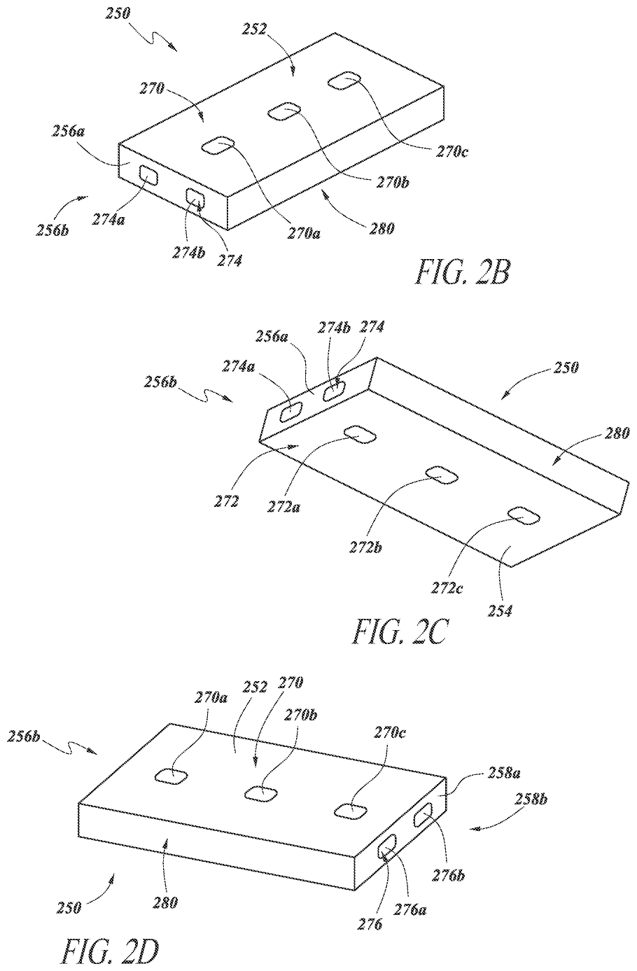

FIG. 2A illustrates the intelligent implant 100 that includes an implantable reporting processor 250 for insertion into the interbody spacer of the intelligent implant 100. The implantable reporting processor 250 can include any of the features described above with respect to implantable reporting processor 150. FIG. 2A illustrates an implantable reporting processor 250 for insertion into the interbody spacer that includes an antenna 260 that does not extend from a surface of the implantable reporting processor 250.

The implantable reporting processor 250, like the implantable reporting processor 150, can include a housing 280 that encloses a battery 290, an electronics assembly 240, an antenna 260, and a plurality of sensors. Similar to the implantable reporting processor 150, the housing 280 of the implantable reporting processor 250 comprises a cover or a casing that encases and secures the various components of the implantable reporting processor 250. For example, as illustrated in FIG. 2A, the implantable reporting processor 250 is in the form of a cartridge for insertion into the patient. The implantable reporting processor 250 can be any shape or size such that it can be inserted into the intelligent implant 100. For example, the implantable reporting processor 250 of FIGS. 2A-2C is thin and wafer shaped to allow it to be inserted through the opening 110 or the opening 112 of the interbody spacer. However, as discussed above with regard to the implantable reporting processor 150, the implantable reporting processor 250 can be cylindrical or any other shape or size. As shown in FIGS. 2A-2C, the implantable reporting processor 250 can include a top surface 252, a bottom surface 254, a medial surface 256a and a lateral surface 258a. The implantable reporting processor 250 can include the housing 280 that covers and encloses the antenna 260 and the electronics assembly 240 at a medial end 256b and the battery 290 at a lateral end 258b of the implantable reporting processor 250.

Similar to the implantable reporting processor 150, the implantable reporting processor 250 can include a plurality of sensors (e.g., ultrasound sensors) positioned on the various surfaces of the implantable reporting processor 250. As shown in FIGS. 2A-2D, the implantable reporting processor 250 can include at least one sensor 270 on the top surface 252 of the implantable reporting processor 250. As illustrated in FIGS. 2A-2C, at least one sensor 270 of the implantable reporting processor 250 are made up of a sensor 270a, a sensor 270b, and a sensor 270c. The sensors 270a, 270b, 270c can be positioned in series along the top surface 252 of the implantable reporting processor 250.

Additionally or alternatively, the implantable reporting processor 250 can include at least one sensor 272 on the bottom surface 254 of the implantable reporting processor 250. As illustrated in FIGS. 2A-2C, at least one sensor 272 can include a sensor 272a, a sensor 272b, and a sensor 272c. The sensors 272a, 272b, 272c can be positioned in series along the bottom surface 254 of the implantable reporting processor 250.

In some configurations, the implantable reporting processor 250 can include at least one sensor 274 on the medial surface 256a of the implantable reporting processor 250. As illustrated in FIGS. 2A-2C, at least one sensor 274 of the implantable reporting processor 250 are made up of a sensor 274a and a sensor 274b. However, at least one sensor 274 can include a greater or fewer number of sensors in any number of arrangements. In some configurations, the implantable reporting processor 250 can include at least one sensor 276 on the lateral surface 258a of the implantable reporting processor 250. As illustrated in FIGS. 2A-2C, at least one sensor 276 of the implantable reporting processor 250 can comprise a sensor 276a and a sensor 276b. However, at least one sensor 276 can include a great or fewer number of sensors in a variety of configurations. In some embodiments, at least one sensor 270, at least one sensor 272, at least one sensor 274, and at least one sensor 276 can be embedded within the material of the housing 280.

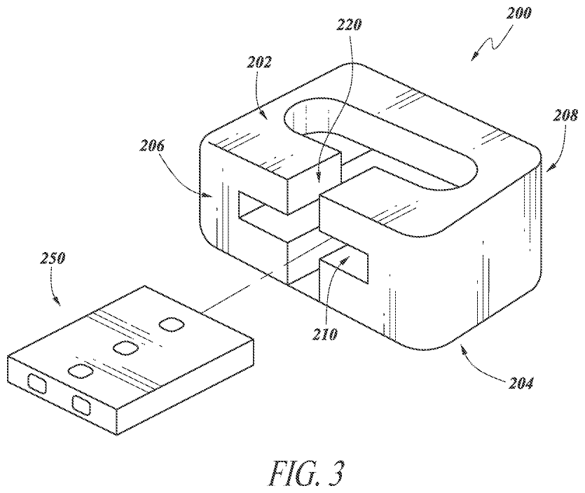

FIG. 3 illustrates an intelligent implant 200 comprising an interbody spacer. The intelligent implant 200 can include any of the features described above with respect to intelligent implant 100. The intelligent implant 200 can include a top surface 202, a bottom surface 204, a medial surface 206, and a lateral surface 208. The intelligent implant 200 includes an opening 210 in the window 220 and an opening 212 in the lateral surface 208. The intelligent implant 200 further includes a window 220 positioned in either the medial end or the lateral end of the intelligent implant 200. In embodiments where the implantable reporting processor includes an antenna that does not extend out of the intelligent implant 200, the window 220 can allow for signals to be transmitted out of the intelligent implant 200 without any interference. This is particularly the case when the housing 280 of the implantable reporting processor 250 comprises a material such as metal. In embodiments where the housing 280 comprises material such as PEEK, signal transmission is not blocked and therefore an intelligent implant 100 without a window can be used.

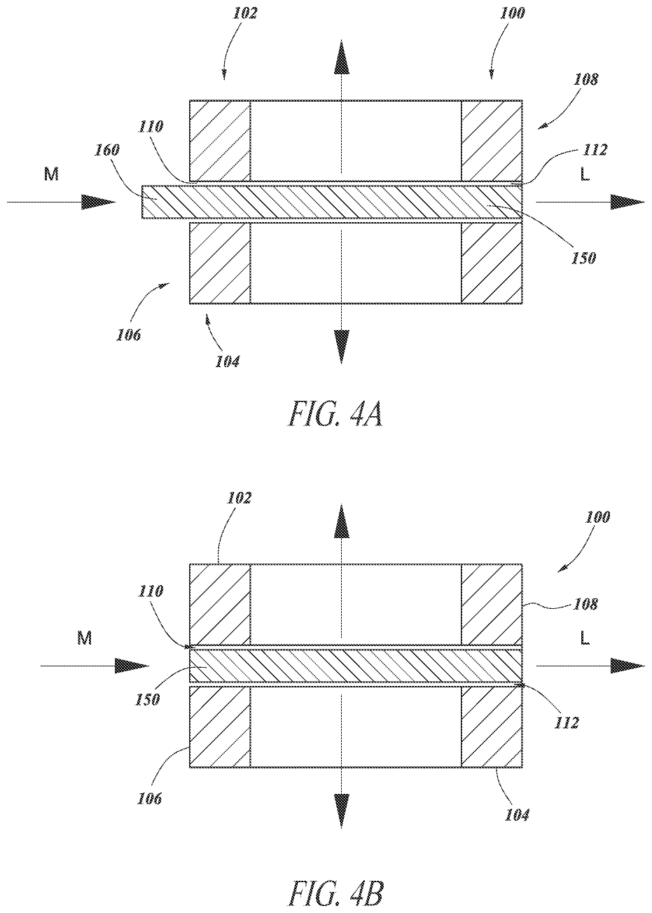

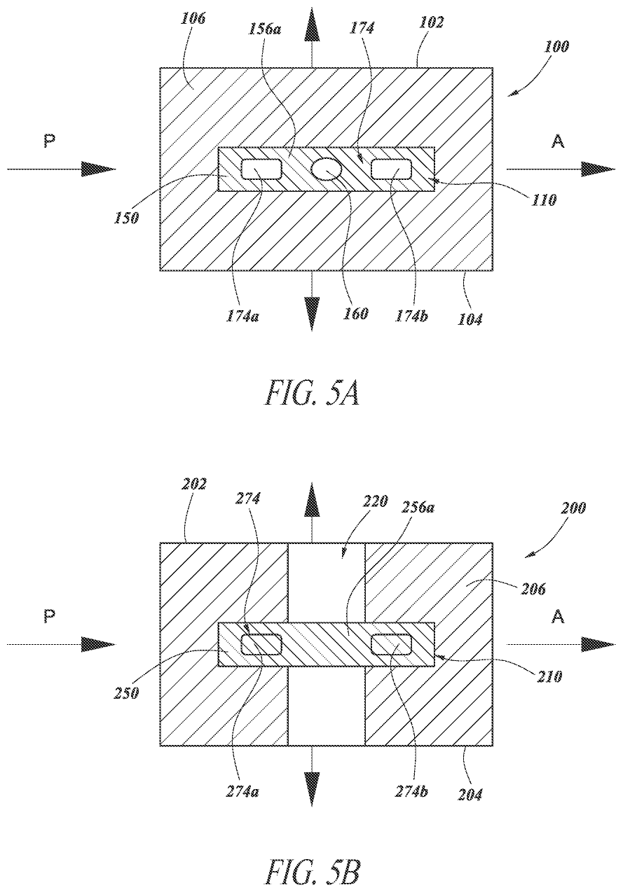

FIGS. 4A-4B illustrate cross-sectional medial-lateral views of the intelligent implant 100 with either the implantable reporting processor 150 or the implantable reporting processor 250 inserted into the interbody spacer. FIG. 4A illustrates a cross-sectional view wherein the antenna 160 extends out of the opening 110 of the medial surface 106. FIG. 4B illustrates a cross-sectional view wherein the entirety of the implantable reporting processor 250 does not extend from either end of the intelligent implant 100 and is contained between the medial surface 106 and the lateral surface 108 of the intelligent implant 100 FIGS. 5A-5B illustrate proximal-anterior views of either the implantable reporting processor 150 or the implantable reporting processor 250 inserted into the intelligent implant 100 or the intelligent implant 200. In FIG. 5B, the window 220 of the implantable reporting processor 250 allows a top and/or bottom portion of the antenna 260 on the medial end 256b of the implantable reporting processor 250 to be exposed. As this is where the antenna 260 is positioned under the housing 280, the antenna 260 can transmit signals out of the intelligent implant 200.

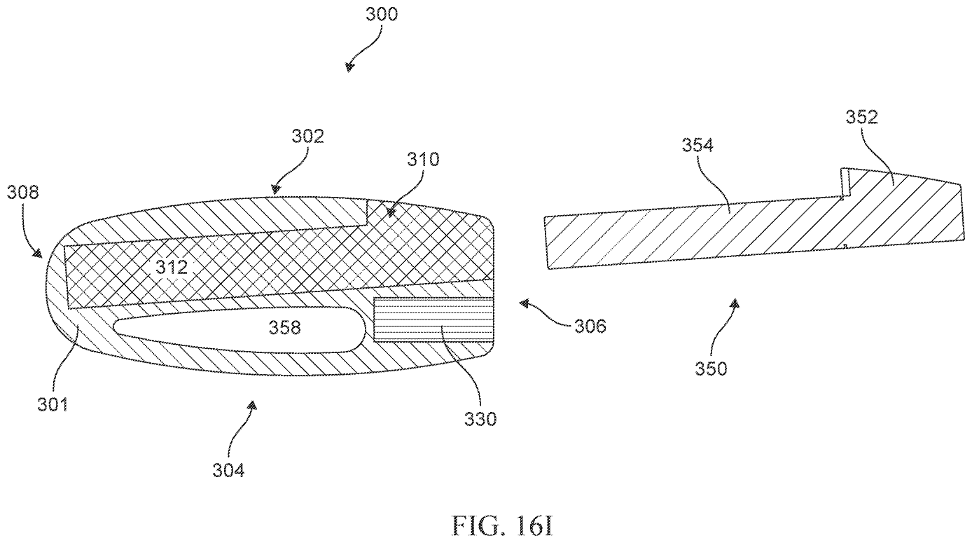

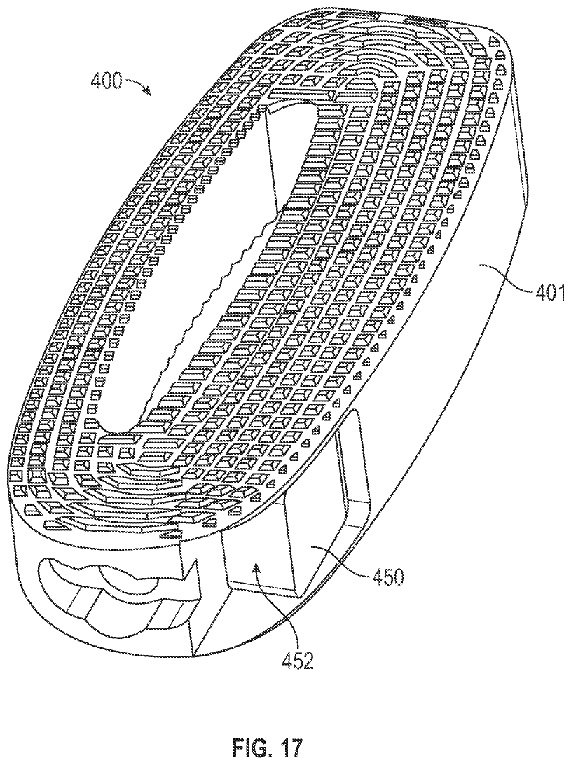

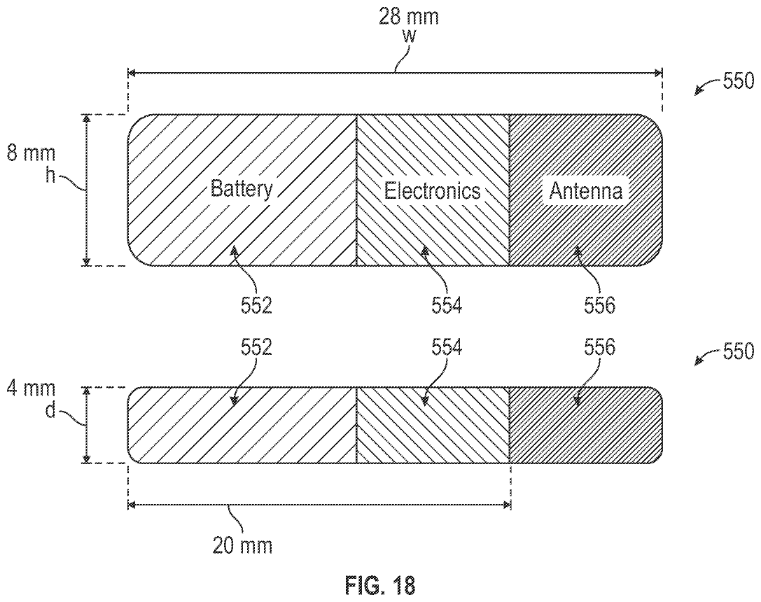

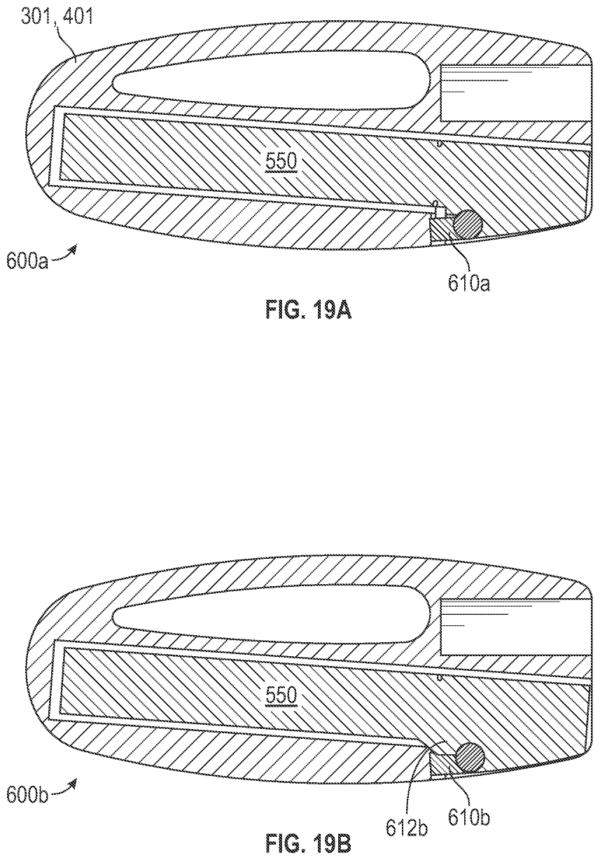

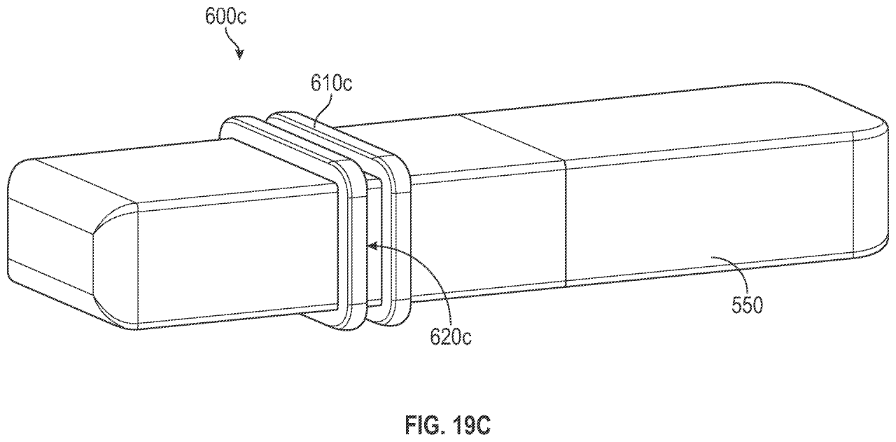

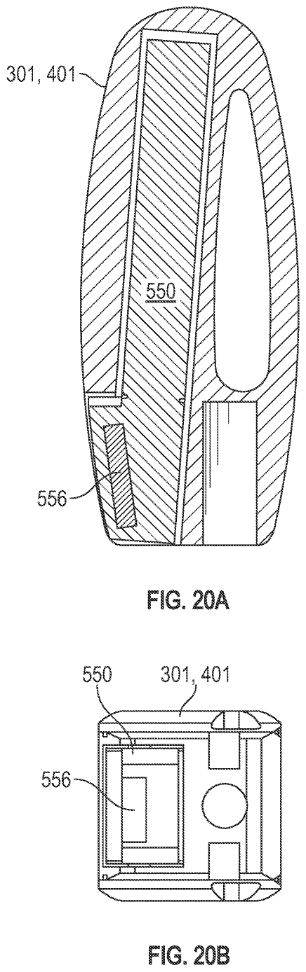

FIGS. 16A-161 illustrates another embodiment of an intelligent implant 300 wherein the intelligent implant 300 is an interbody spacer 301. In some embodiments, the interbody spacer 301 of FIGS. 16A-161 is a PLIF implant. The intelligent implant 300 can include an implantable reporting processor 350 for insertion into the interbody spacer 300. The implantable reporting processor 350 can include any of the features described above with respect to the implantable processor 150, 250. The implantable reporting processor 350 can be inserted into the interbody spacer 301 as shown in FIGS. 16H-161.

As illustrated in FIGS. 16A-16B, the interbody spacer 301 can include a left surface 302, a right surface 304, a first end 306, a second end 308, a bottom surface 305, and a top surface 303. In some embodiments, depending on how the interbody spacer 301 is inserted into the body, the bottom surface 305 can form an inferior side of the interbody spacer 301 and the top surface 303 can form a superior side of the interbody spacer 301 or, alternatively, the bottom surface 305 can form an inferior side of the interbody spacer 301 and the bottom surface 305 can form a superior surface of the interbody spacer 301. The first end 306 can include an opening 310 that allows the implantable reporting processor 350 to be inserted into the interbody spacer 301. As shown in the cross-sectional views of FIGS. 16H-161, the interbody spacer 301 can include a cavity 312 that extends internally through the body 301 from the opening 310 at the first end of the body 301 to the second end of the body 301. The opening 310 and the cavity 312 can allow the implantable reporting processor 350 to be inserted and secured within the interbody spacer 300. In some examples, the interbody spacer 301 can be made of PEEK. The interbody spacer 301 can also include a tooling hole 330 on the first end 306 of the interbody spacer 301. The interbody spacer 301 can also include a plurality of tooling cutouts 340a, 340b at the first end 306 of the interbody spacer 301. The tooling hole 330 and cutouts 340a, 340b allow the physician to insert the interbody spacer 301 into the spine of the patient.

In other examples, interbody spacer 301 can be made of titanium. The intelligent implant 300 can be configured to be inserted into any location of the spine. For example, the intelligent implant 300 can comprise any of a lumbar interbody spacer, a cervical interbody spacer, or a thoracic interbody spacer.

In some embodiments, the interbody spacer 301 can include a plurality of cutouts and openings that better allow the physician to fill the interbody spacer with material to help retain the intelligent implant 300 and provide for enhanced fusion between adjacent vertebrae. For example, the interbody spacer 301 can be filled with body material (e.g., bloody sawdust) and other biological material or synthetics. As illustrated in FIGS. 16A-16H, the interbody spacer 301 can include an opening 350 that forms a throughhole that extends from an anterior side of the interbody spacer 301 to a posterior side of the interbody spacer. As mentioned, the opening can be filled with biological or synthetic materials to aid in the fusion of adjacent vertebrae.

The interbody spacer 301 can include ridged or grooved surfaces that provide scoring to better allow the intelligent implant 300 to be secured between vertebrae in the body. As shown in FIGS. 16A-16B, the top surface 303 includes a plurality or alternating ridges 303a and grooves 303b. Similarly, the bottom surface 305 can also include a plurality of alternating ridges 305a and grooves 305b. The angled scoring formed on the top surface 303 and the bottom surface 305 increase the surface area of the interbody spacer 301 engaging the adjacent vertebrae and provide for better securement of the intelligent implant 300 within the body.

The implantable reporting processor 350 can include a housing 380 that encloses numerous elements for measuring patient kinematics and powering the intelligent implant 300. For example, the housing 380 can enclose a battery, an electronics assembly, an antenna, and one or more sensors. The one or more sensors can include any of the sensors described herein. The housing 380 can comprise a cover or a casing that encases and secures the various components of the implantable reporting processor 350. As shown in FIG. 161, the implantable reporting processor 350 can be in the form of a cartridge for insertion into the intelligent implant 300. The cartridge can be any shape or size such that it can be inserted into the interbody spacer 301. In the embodiment presently illustrated, the implantable reporting processor 350 is shaped to fit within the opening 310 and the cavity 312 of the interbody spacer 301. In some examples, the implantable reporting processor 350 can have a first portion 352 and a second portion 354, wherein the first portion 352 is larger than the second portion 354. The second portion 354 of the implantable reporting processor 350 can be sized such that it can be received within the cavity 312 of the interbody spacer 301. The first portion 352 of the implantable reporting processor 350 can be sized such that it can be received within the opening 310 but not extend into the cavity 312. This can allow the implantable reporting processor 350 to be secured and correctly positioned within the body of the interbody spacer 301.

The implantable reporting processor 350 can be in the form of a cartridge that is insertable into the interbody spacer 301 through the opening 310 of the first end 306. In some embodiments, the implantable reporting processor 350 can form a positive connection with the interbody spacer 301 before the interbody spacer 301 is inserted into the patient. For example, the implantable reporting processor 350 has a locking mechanism (e.g., a tab) that can lock with the interbody spacer 301. positive connection can be any of a number of mechanical connections such as snap fits, locks, twists, mated threads, etc. In some embodiments, the implantable reporting processor 150 can be inserted into the interbody spacer reversibly. The reversible connection between the implantable reporting processor 150 and the interbody spacer can provide the physician with the option of removing the cartridge in situations where any maintenance may need to be performed on the implantable reporting processor 150 once it is inserted into the patient. A reversible connection can allow the implantable reporting processor 150 to be removed in the instance when a battery may need to be replaced. In other embodiments, the locking mechanism is not reversible. The implantable reporting processor can include a screw that can allow the implantable reporting processor 350 to be assembled with the interbody spacer 301 before the intelligent implant 300 is implanted.

In some embodiments, the implantable reporting processor 350 can be configured to communicate with external devices. Communication can occur, for example, using Bluetooth Low Energy, to transmit collected data or receive programming and configuration data. In some embodiments, the implantable reporting processor can include an antenna that can be optimized for frequencies in the range of 2.4 to 2.6 GHz.

As is discussed elsewhere in the application, the implantable reporting processor 350 can include one or more sensors positioned on a surface of the implantable reporting processor 350. In some examples, the at least one sensor can be hermetically sealed and mounted inside or attached to a surface of the implantable reporting processor (e.g., on the top surface of the implantable reporting processor 350).

In some examples, the implantable reporting processor 350 can include strain or force sensors to detect strain or force on the surface of the implantable reporting processor 350 as a way to detect fusion between adjacent vertebrae in which the interbody spacer is positioned because load increases as fusion progresses.

In some embodiments, the at least one sensor of the implantable reporting processor 350 can include a vibration sensor to detect acoustic emissions associated with scratching/abrasion of the interbody spacer 301 against adjacent vertebrae. The extent of acoustic emissions can change (e.g., likely decreasing) as intervertebral fusion progresses. In some embodiments, the vibration sensor can be combined with at least one accelerometer to collect acoustic emission/vibration measurements when the patient is in known activities such as walking. The accelerometer can be a low power AC or DC accelerometer. The at least one accelerometer can be hermetically sealed within the implantable reporting processor 350. In some embodiments, the at least one accelerometer can measure at least one of the inclination angle of the spine relative to gravity vector, the spine angle relative to the gravity vector which provides a center of mass measurement, and the center of mass measurement which correlates to the recover, pain level, and/or health status of the patient. The at least one accelerometer can measure activity patterns of the patient (e.g., walking) and trigger the collection of data by the at least one accelerometer to occur during targeted activities (e.g., walking).

As discussed above, an implantable reporting processor, in the form of a cartridge, can be inserted into the intelligent implant. The cartridge can be inserted into the interbody spacer of the intelligent implant such that it does not extend beyond a perimeter of the interbody spacer's geometry. This can prevent any interference of the cartridge with surrounding soft tissue and neurological tissue (i.e., the spinal cord). This can be seen in FIGS. 16A-161 which illustrates the implantable reporting processor cartridge 350 inserted into the PLIF interbody spacer 301. As illustrated in the cross-sectional view of FIG. 16H, the first portion of the cartridge 350 can lay flush with a perimeter of the PLIF interbody spacer 301. In another embodiment, shown in FIG. 17, the implantable reporting processor cartridge 450 is inserted into a LLIF interbody spacer 401. As shown, a medial end 452 of the cartridge 450 does not lie flush with a perimeter of the LLIF interbody spacer 401, but extends within the perimeter.

In some embodiments, the cartridge 350, 450 can have an outer wall that is configured to provide fracture/yield strengths in order to sustain the expected load on the interbody spacer 301, 401. The outer wall of the cartridge 350, 450 can have a minimum wall thickness of 0.5 mm. In some examples, the wall thickness of the cartridge 350, 450 can be less than 1 mm. In some embodiments, the wall thickness can be 0.4 mm, about 0.5 mm, about 0.6 mm, about 0.7 mm, about 0.8 mm, about 0.9 mm, about 1.0 mm, about 1.1 mm, about 1.2 mm. In some examples, the wall thickness can range between about 0.4 mm-0.6 mm, between about 0.45 mm-0.55 mm, between about 0.9 mm- 1.10 mm, between about 0.95 mm- 1.05 mm, between about 0.5 mm- 0.6 mm, between about 0.6 mm-0.7 mm, between about 0.7 mm-0.8 mm, between about 0.8 mm-0.9 mm, between about 0.9 mm-1.0 mm, and any value in between those ranges listed, including endpoints.

FIG. 18 illustrates an embodiment of a cartridge 500. In some embodiments, the cartridge 550 can be rectangular, however the cartridge can be any shape (e.g., cylindrical). The cartridge 550 can have a 1:2 width to height aspect ratio that can provide for optimized mechanical strength for handling compressive strengths. In some embodiments, the cartridge 550 can have a 2:3 width to heigh aspect ratio which can also provide for mechanical strengths. In some embodiments, the cartridge 550 can have a width to height aspect ratio that is 4:12, 5:12, 6:12, 7:12, 8:12, 9:12, 10:12, between about 4:12-5:12, between about 5:12-6:12, between about 6:12-7:12, between about 7:12-8:12, between about 8:12-9:12, between about 9:12-10:12, and any aspect ratio in between those ranges listed, including end points. In some examples, the width to height aspect ratio can provide mechanical strength for handling compression shear and torsion forces.

As shown in FIG. 18, in some embodiments the cartridge 500 can include a power source such as a batter 552, electronics 554, and an antenna 556. In some embodiments the cartridge 550 can have a height h of approximately 8 mm, a width w of approximately 28 mm, and a distance d of approximately 4 mm. In some embodiments, the length of the cartridge 550 without the antenna is approximately 20 mm.

The implantable reporting processor, in the form of a cartridge, is insertable into the intelligent implant. This feature allows the implantable reporting processor cartridge to be used in various applications and in implants of varying sizes. In some embodiments, the intelligent implant can include a locking structure to retain the implantable reporting processor cartridge within the interbody spacer such that it does not fall out or loosen once the intelligent implant is inserted into the patient's body.

In addition to the embodiments discussed about regarding the securement of the cartridge, FIGS. 19A, 19B, and 19C illustrate various examples of mechanical locking structures. FIG. 19A illustrates a locking mechanism 600a that includes an interbody locking finger 610a. The interbody locking finger 610a of the locking mechanism 600a can be a passive locking spring finger that is formed on the interbody spacer that can deform outward on the interbody as the cartridge 550 is inserted. In some embodiments, the cartridge design could have a mechanical ramp and ledge, such that as the cartridge 550 is inserted, the interbody locking finger 610a deforms outward until the cartridge 550 is fully inserted, and the interbody locking finger 610a moves back into place. In some embodiments, the locking mechanism 600 a can form a minimum 0.5 mm- 1 mm mechanical interference that can retain the cartridge 550 after insertion. In some examples, the interbody locking finger 610a can be manufactured by additive manufacturing as a feature contained within the interbody spacer.

FIG. 19B illustrates a locking mechanism 600b that can include a locking pin 610b and a cartridge locking ledge 612b. In some embodiments, the locking mechanism 600b forms a passive, locking ledge design on the cartridge 550. The locking mechanism 600b is configured to retain the cartridge 550 within the interbody spacer by the locking pin 610b on the interbody spacer. In some embodiments, the locking pin 610b of the cartridge can have a minimum 0.5 mm-1.0 mm mechanical interference to retain the cartridge 550 after insertion within the interbody spacer. In some embodiments, the locking ledge 612b of the cartridge 550 can have the radius of the locking pin 610b. In some examples, the locking pin 610 b may be an inserted pin/rod or a 3D printed feature contained within the interbody spacer.

FIG. 19C illustrates a locking mechanism 600c that is positioned on the body of the cartridge 550. As shown, the locking mechanism 600c can include a locking clip 610c and groove 620c. The locking clip 610c is configured to reduce its profile when compressed and expand its profile when decompressed. In some embodiments, the interbody spacer can include a groove (not illustrated). The locking clip 610c on the cartridge 550 can compress as it is inserted into the interbody spacer, but then expand/decompress when the cartridge 550 is inserted such that the locking clip 610c is positioned within the groove of the interbody spacer.

In some embodiments, after the cartridge is assembled within an interbody spacer, an antenna can be configured to wirelessly communicate with an outside source to transmit data. In some embodiments, the cartridge is fully contained within the interbody spacer while the antenna has a line-of-sight uninterrupted by the interbody spacer. FIGS. 20A and 20B illustrate an embodiment of a cartridge 550 positioned within an interbody spacer 301,401 wherein the cartridge is contained within the interbody spacer 301, 401. As shown, each of the cartridge 550 includes an antenna 556 that is positioned within the outer housing of the cartridge and within the opening of the interbody spacer such that the antenna 556 can transmit signals uninterrupted. Thie can provide for optimal radio frequency efficiency, electric field strength, and radiation. In some embodiments, we want to position the antenna 556 as far away as possible from the Titanium material while maintaining the cartridge 556 fully contained within the perimeter of the interbody spacer 301,401. In some embodiments, the cartridge can have a 0.2 mm clearance around the cartridge 550 within the interbody spacer 301, 401.

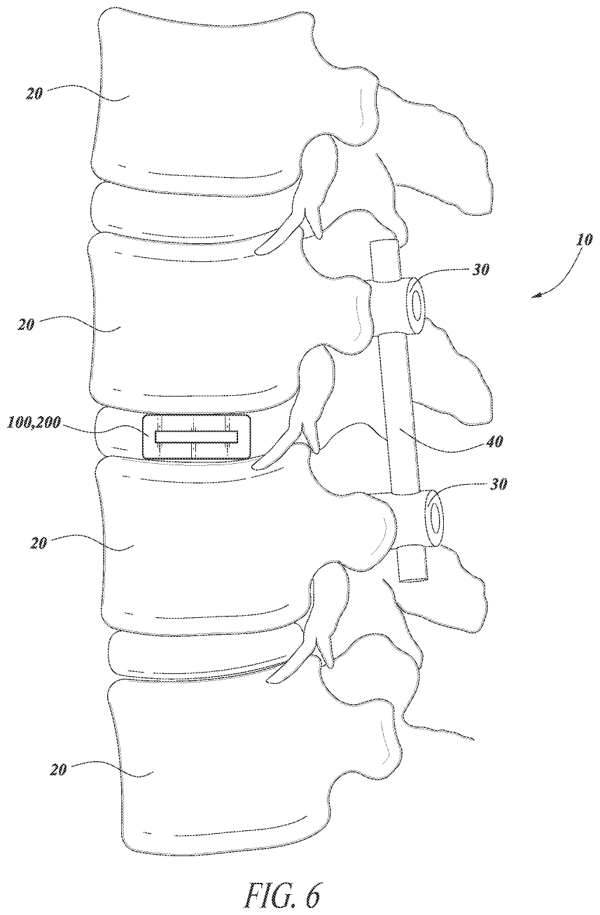

FIG. 6 illustrates a posterior-anterior view of the intelligent implant 100, 200 inserted into the spine 10 of a patient during spinal fusion. As discussed above, the intelligent implant 100, 200 is in the form of an interbody spacer and is inserted between adjacent vertebrae 20. The adjacent vertebrae 20 include a plurality of pedicle screws 30 and rod 40 that help to secure the vertebrae 20 in place during and after spinal fusion.

### Implantable Reporting Processors (IRP)

The present disclosure provides implantable reporting processors (IRP) or cartridges for implant systems for use in spinal procedures. As previously mentioned, the IRP can be a component assembly that is manufactured independent of other components of the implant system and later assembled together with a component of an implant system. But in other configurations, the IRP can be integrated with a component of the implant system during manufacture of the component. When integrated, the IRP can still include any features of the separate cartridge described herein. In some embodiments, the present disclosure provides a reporting processor that is intended to be implanted with a medical device, e.g., a prosthesis, where the reporting processor monitors the state of the device after implantation, typically by obtaining kinematic data in the range of about 10-120 Hz.

As discussed herein, the state of the device may include the integrity of the device, the movement of the device, the forces exerted on the device and other information relevant to the implanted device. The present disclosure also provides medical devices having a structure such that they can be readily fitted with an IRP. An implantable medical device that has been fitted with an IRP is referred to herein as an intelligent implant, in recognition that the implant is monitoring its own state or condition to thereby obtain data, where that data is stored in the implant and then as needed, that data is transmitted to a separate device for review by, e.g., a physician.

For example, an intelligent implant of the present disclosure having suitable internal electronic components can be utilized to monitor and measure the movements of a surgical patient's movements and kinematics after surgery (e.g., activity, gait, steps, posture, lumbar range of motion, etc.) after the interbody spacer or cage is implanted during spinal fusion, store the measurement data and unique identification information of the prosthetic components, and transfer the data to an external recipient (e.g., doctor, clinician, medical assistant, etc.) as required. The IRP will include one or more sensors, such as gyroscopes, accelerometers, and temperature and pressure sensors, and these sensors may be located anywhere within the IRP outer casing, e.g., they may all be located on the PC board. In some embodiments, e.g., when the intelligent implant is a secured within an interbody spacer or cage, the IRP makes kinematic measurements, and in another embodiment the IRP makes only kinematic measurements. Thus, an intelligent implant may include sensors for kinematic measurements, to determine the movements experienced by the implanted prosthesis.

The IRP and the medical device are each intended to be implanted into a living subject, e.g., a mammal, e.g., a human, horse, dog, etc. Accordingly, in some embodiments the IRP is sterile, e.g., is treated with sterilizing radiation or is treated with ethylene oxide. In some examples, the intelligent implant comprising the IRP and the medical device is sterile, again optionally by treatment with sterilizing radiation or ethylene oxide, as two examples. In order to be protected from the in vivo environment, in some embodiments the IRP is hermetically sealed, so that fluids cannot enter the IRP.

In some embodiments, the implantable device needs to be sturdy as well as small or space-efficient because of the limited space within the body and/or within the prosthetic implant to place such devices. Challenges to the commercial success of an implantable device with internal electronic components and either internal or external transmitting antennae are that the devices and/or the transmitting antennae should not be unsuitably large, their power consumption should allow them to operate for a suitably long period of time, i.e., not for limited durations, and they should not be adversely affected by their local biologic environment. An IRP of the present disclosure may have suitable internal or external space-efficient and/or power-efficient antennae.

The IRP typically comprises an outer casing that encloses a plurality of components. Exemplary suitable IRP components include a signal portal, an electronics assembly, and/or a power source. The signal portal functions to receive and transmit wireless signals, and may contain, for example, an antenna for transmitting the wireless signals. The electronics assembly includes a circuit assembly which may comprise, e.g., a PC board and electrical components formed on one or more integrated circuits (ICs) or chips, such as a radio transmitter chip, a real-time clock chip, one or more sensor components, e.g., an Inertial Measurement Unit (IMU) chip, temperature sensor, pressure sensor, tilt sensor, strain sensor, pedometer, a memory chip, and the like. In addition, the electronics assembly may include a header assembly which provides a communication interface between the circuit assembly and the signal portal (e.g., antenna). The power source provides the energy needed to operate the IRP, and may be, for example, a battery. The IRP will also include one or more sensors, such as gyroscopes, accelerometers, pedometers, tilt sensors, strain sensors, and temperature and pressure sensors, and these sensors may be located anywhere within the IRP outer casing, e.g., they may all be located on the PC board. More precisely, an embodiment of the present disclosure is directed to space-efficient, printed circuit assemblies (PCAs) for an implantable reporting processor (IRP). The implantable reporting processor may also include a plurality of transmitting antennae structured in different configurations. As such, an embodiment of the present disclosure is directed to a plurality of enhanced space-efficient and power-efficient antenna configurations for an implantable reporting processor, such as an IRP.

An example of an implantable reporting processor includes an outer casing, or housing, sized to fit in, or to form a part of, an implantable prosthesis that has at least a portion designed to fit in a bone of a living patient. Electronic circuitry is disposed in the housing and is configured to provide, to a destination outside of a patient's body, information related to the prosthesis. The battery is also disposed in the housing and is coupled to the electronic circuitry.

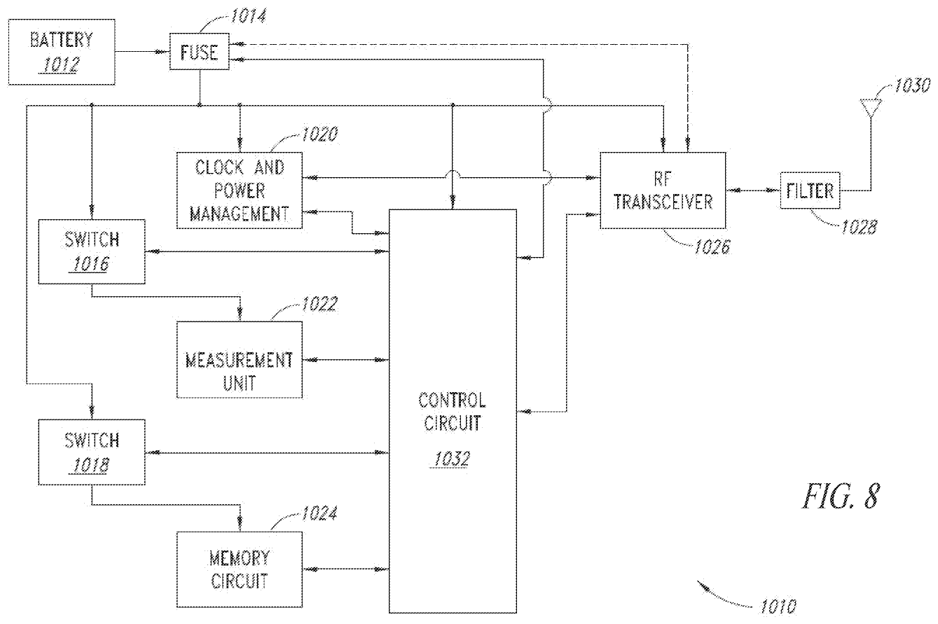

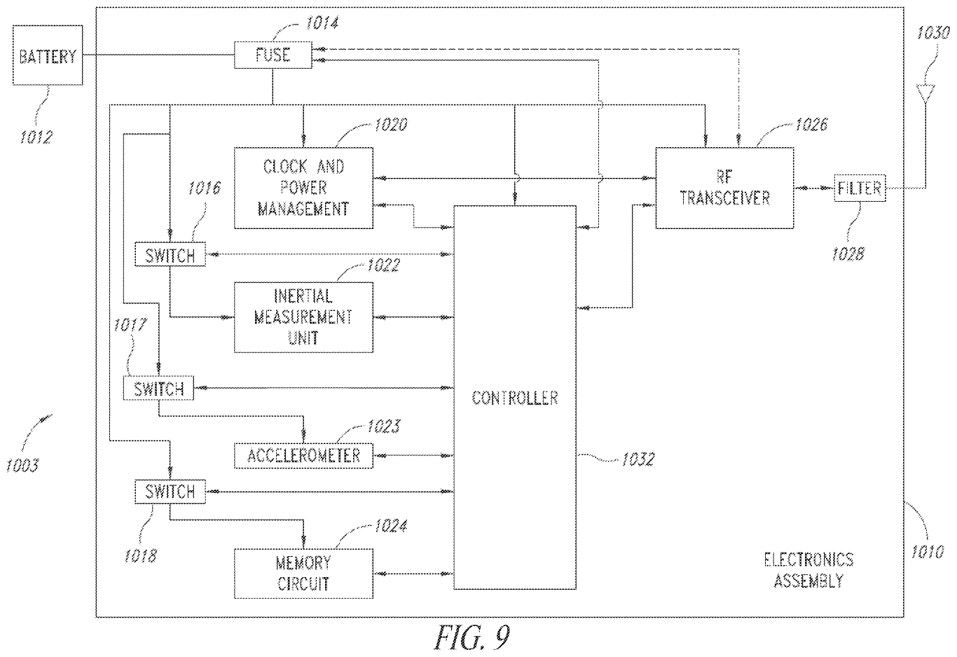

FIGS. 8 and 9 illustrate block diagrams of IRPs that may be associated with an intelligent implant. The components include a battery 1012, an RF antenna 1030, and an electronics assembly 1010 having various circuitry powered by the power source. Depending on the design, the circuitry of the electronics assembly 1010 includes a fuse 1014, one or more switches 1016, 1017, 1018, a clock and power-management circuit 1020, one or more measurement units 1022, accelerometer 1023, memory 1024, communication components, e.g., a radio-frequency (RF) transceiver 1026 and an RF filter 1028, that couple with the RF antenna 1030, and/or a controller 1032. Not shown but optionally present is a tilt sensor. Also not shown but optionally present is a strain sensor.

### Measurement Unit

### Overview

In some embodiments, the measurement unit can monitor one or more aspects of the implant. The measurement unit 1022 may be configured to detect, measure and/or monitor information relevant to the state of the device after implantation. The state of the device may include the integrity of the device, the movement of the device, the forces exerted on the device and other information relevant to the implanted device. This type of measurement unit 1022 may include a processor located on a printed circuit board of the electronics assembly 1010 and one or more sensors, such as gyroscopes, accelerometers, tilt sensors, strain sensors, and temperature and pressure sensors coupled to the processor. These sensors may also be located on a printed circuit board of the electronics assembly 1010. Alternatively, some or all these sensors, e.g., a tilt sensor, a strain sensor, a gyroscope, an accelerometer, may be in or on another structure of the intelligent implant separate from the electronics assembly 1010.

In some embodiments, the measurement unit can monitor one or more aspects of body or body segment/joint condition or function (e.g., healing, motion including measurement of the positions, angles, velocities, and accelerations of body segments and joints). The measurement unit 1022 may be configured to detect, measure and/or monitor information relevant to the state of a body or body segment after implantation of the device. The state of the body or a body segment may include, for example, kinematic information of the body or a body segment, healing information. This type of measurement unit 1022 may include a processor located on a printed circuit board of the electronics assembly 1010 and one or more sensors, such as gyroscopes, accelerometers, tilt sensors, strain sensors, electrodes, and temperature and pressure sensors coupled to the processor. These sensors may also be located on a printed circuit board of the electronics assembly 1010. Alternatively, some or all these sensors may be in or on another structure of the intelligent implant device separate from the electronics assembly 1010.

In some embodiments, can monitor body tissue (e.g., anatomy, physiology, metabolism, and/or function). The measurement unit 1022 may be configured to detect, measure and/or monitor information relevant body tissue after implantation of the device. The body tissue monitoring may include, for example, pressure or pH level. This type of measurement unit 1022 may include a processor located on a printed circuit board of the electronics assembly 1010 and one or more sensors, such as fluid pressure sensors, fluid volume sensors, pulse pressure sensors, blood volume sensors, blood flow sensors, chemistry sensors (e.g., for blood and/or other fluids), metabolic sensors (e.g., for blood and/or other fluids). These sensors may also be located on a printed circuit board of the electronics assembly 1010. Alternatively, some or all these sensors may be in or on a structure of the intelligent implant device separate from the electronics assembly 1010.

The measurement unit may perform one or more of any of the above-described intelligence functions.

Inertial measurement unit

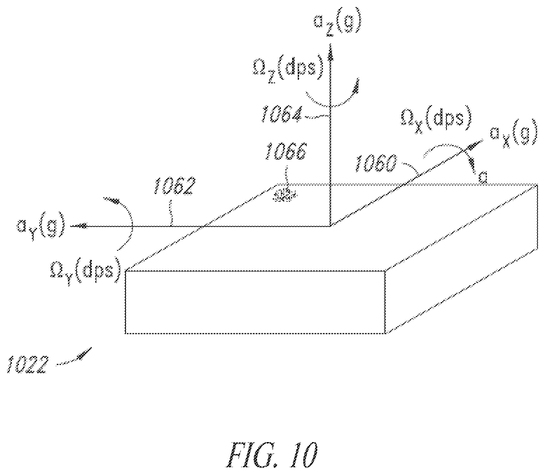

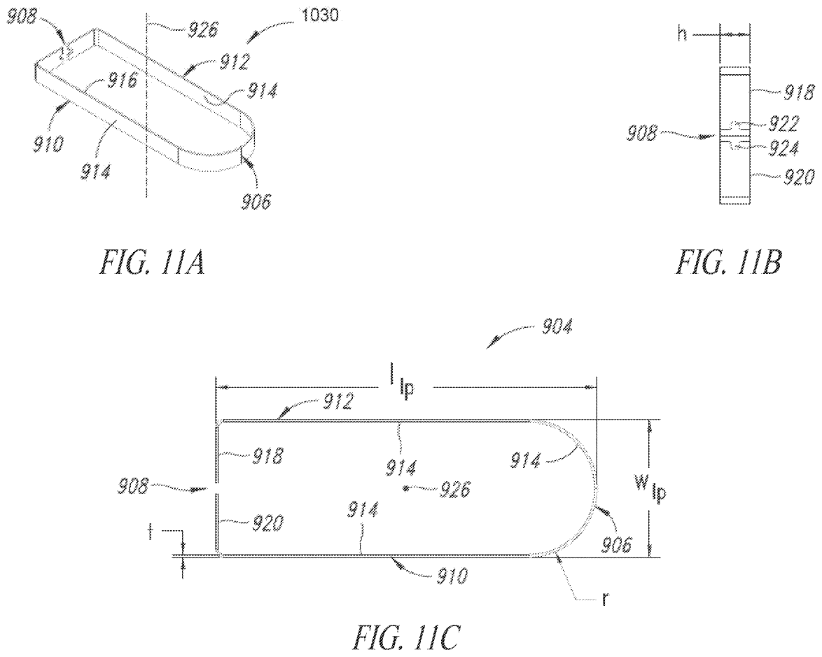

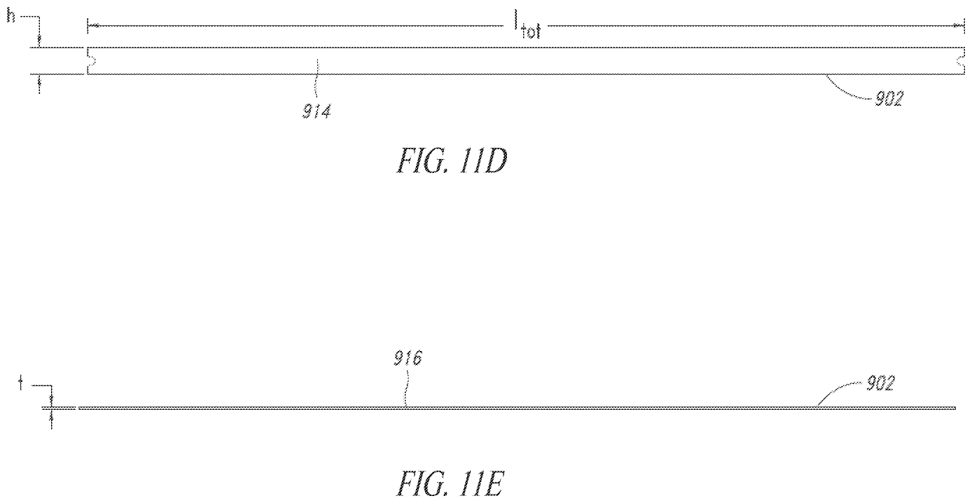

In some embodiments, the measurement unit 1022 is an inertial measurement unit (IMU). For example, the IMU can be a Bosch BMI 160 small, low-power, IMU. As shown in FIG. 10, the measurement unit 1022 includes three measurement axes 1060, 1062, and 1064, which for purposes of description, are arbitrarily labeled x, y, z. That is, in a Cartesian coordinate system, the labels “x,” “y,” and “z” can be applied arbitrarily to the axes 1060, 1062, and 1064 in any order or arrangement. A mark 1066 is a reference that indicates the locations and orientations of the axes 1060, 1062, and 1064 relative to the IMU package. The IMU can include one or more accelerometers, for example three accelerometers, each of which senses and measures a linear acceleration a(t) along a respective one of the axes 1060 (x), 1062 (y), and 1064 (z), where ax(t) is the acceleration along the x axis, ay(t) is the acceleration along the y axis, and az(t) is the acceleration along the z axis. Each accelerometer generates a respective analog sense or output signal having an instantaneous magnitude that represents the instantaneous magnitude of the sensed acceleration along the corresponding axis. For example, the magnitude of the magnitude of the accelerometer output signal at a given time is proportional the magnitude of the acceleration along the accelerometer's sense axis at the same time.

The IMU can include one or more gyroscopes, for example three gyroscopes, each of which senses and measures angular velocity Q(t) about a respective one of the axes 1060 (x), 1062 (y), and 1064 (z), where Qx(t) is the angular velocity along the x axis, Qy(t) is the angular velocity along the y axis, and Qz(t) is the angular velocity along the z axis. Each gyroscope generates a respective analog sense or output signal having an instantaneous magnitude that represents the instantaneous magnitude of the sensed angular velocity about the corresponding axis. For example, the magnitude of the gyroscope output signal at a given time is proportional the magnitude of the angular velocity about the gyroscope's sense axis at the same time.

The IMU can one or more analog-to-digital converters (ADCs) for each axis 1060, 1062, and 1064, for example one ADC for converting the output signal of the corresponding accelerometer into a corresponding digital acceleration signal, and another ADC for converting the output signal of the corresponding gyroscope into a corresponding digital angular-velocity signal. For example, each of the ADCs may be an 8-bit, 16-bit, or 24-bit ADC.

Each ADC may be configured to have respective parameter values that are the same as, or that are different from, the parameter values of the other ADCs. Examples of such parameters having settable values include sampling rate, dynamic range at the ADC input node(s), and output data rate (ODR). One or more of these parameters may be set to a constant value, while one or more others of these parameters may be settable dynamically (e.g., during run time). For example, the respective sampling rate of each ADC may be settable dynamically so that during one sampling period the sampling rate has one value and during another sampling period the sampling rate has another value.

For each digital acceleration signal and for each digital angular-velocity signal, the IMU can be configured to provide the parameter values associated with the signal. For example, the IMU can provide, for each digital acceleration signal and for each digital angular-velocity signal, the sampling rate, the dynamic range, and a time stamp indicating the time at which the first sample or the last sample was taken. The IMU can be configured to provide these parameter values in the form of a message header (the corresponding samples form the message payload) or in any other suitable form.

### Ultrasound

In some embodiments, the IRP can include one or more ultrasound sensors. As will be discussed in more detail below, ultrasound sensors provide for a direct measurement of subsidence of vertebrae and migration of the intelligent implant after spinal fusion.

Ultrasound sensors can be useful as they can take measurements using sound waves that are not impacted by materials or tissue overgrowth. As illustrated above in FIGS. 1A-1C and 2A-2B, in some embodiments, the ultrasound sensors can be positioned on a surface of the intelligent implant.

Ultrasound sensors used can be either M-mode or B-mode ultrasound sensors. In some embodiments, the ultrasound sensors are B-mode ultrasound sensors that are configured to measure distance. As will be discussed in more detail below, the ultrasound sensors positioned about the implantable reporting processor can provide direct measurements of various conditions of the spinal fusion. For example, the sensors (e.g., at least one sensor 174, at least one sensor 176, at least one sensor 274, at least one sensor 276) positioned on the medial and lateral ends of the implantable reporting processor can provide direct measurements of migration of the interbody spacer or spinal cage. This can be useful as it can help to determine whether adjacent vertebrae have fused. If the interbody spacer or spinal cage continues to migrate, this can be an indication to the physician that spinal fusion has not fully occurred.

As another examples, the sensors (e.g., at least one sensor 170, at least one sensor 172, at least one sensor 270, at least one sensor 272) positioned on the top surface and the bottom surface of the implantable reporting processor can provide direct measurements of subsidence of adjacent vertebrae. The sensors positioned on the top surface and the bottom surface of the implantable reporting processor measures the distance between the interbody spacer or spinal cage and adjacent vertebrae post insertion and thereafter as a function of time. As the distance between a top surface of the interbody spacer or spinal cage and a first vertebrae and/or a distance between a bottom surface of the interbody spacer or spinal cage and a second vertebrae changes over time, this can indicate to the physician anatomical changes due to loading and/or bone quality that could result in pain associated with spinal deformation.

In some embodiments, the ultrasound sensor can be ultra-low-power ultrasound sensors that can be drive in the microAmps range.

### Fuse

In some embodiments, the fuse 1014 can be any suitable fuse (e.g., permanent) or circuit breaker (e.g., resettable) configured to prevent the battery 1012, or a current flowing from the battery, from injuring the patient and damaging the battery and one or more components of the electronics assembly 1010. For example, the fuse 1014 can be configured to prevent the battery 1012 from generating enough heat to burn the patient, to damage the electronics assembly 1010, to damage the battery, or to damage structural components of the intelligent implantable implant.

### Communication

RF telemetry

The RF transceiver 1026 can be a transceiver that is configured to allow the controller 1032 (and optionally the fuse 1014) to communicate with a base station (not shown in FIG. 8) configured for use with the intelligent implant. For example, the RF transceiver 1026 can be any suitable type of transceiver (e.g., Bluetooth, Bluetooth Low Energy (BTLE), and WiFi®), can be configured for operation according to any suitable protocol (e.g., MICS, ISM, Bluetooth, Bluetooth Low Energy (BTLE), and WiFi®), and can be configured for operation in a frequency band that is within a range of 1 MHz-5.4 GHz, or that is within any other suitable range.

In some embodiments, the filter 1028 can be any suitable bandpass filter, such as a surface acoustic wave (SAW) filter or a bulk acoustic wave (BAW) filter.

### Antenna—Overview

The antenna 1030 can be any antenna suitable for the frequency band in which the RF transceiver 1026 generates signals for transmission by the antenna, and for the frequency band in which a base station (not shown in FIG. 8 or 9) generates signals for reception by the antenna.

### Loop Antenna

In some embodiments, the antenna 1030 can be a loop antenna. For example, the loop antenna can be a conductive loop formed of platinum-iridium (PtIr=90/10) ribbon with one end connected to the radio transceiver and the other end connected to the battery reference potential (GND). The loop antenna provides a magnetic loop, e.g., AC signal in a conductive loop generates magnetic field. The antenna can be encapsulated by the cover and epoxy backfill, both of which are electrically non-conductive. The antenna can be the only electrically active component of the implantable reporting processor outside the hermetic assembly and under normal operating conditions is insulated by the epoxy backfill and PEEK cover from interacting electrically with surrounding tissue.

FIGS. 11A-11E illustrates a plurality of views of a loop antenna that can be configured to be used with the IRPs and integrated IRPs. The loop antennas can be designed to transmit information generated by the electronics assembly of the IRP to a remote destination outside of the body of a subject in which the intelligent implant is implanted, and to receive information from a remote source outside of the subject's body. The loop antenna can be a flat ribbon 902 configured in a loop 904 having a curved end 906, a flat end 908 opposite the curved end, and a pair of opposed sides 910, 912 that extend between the curved end and the flat end. The flat ribbon 902 forming the loop antenna has a major surface 914 and a minor edge 916. Due to skin-effect on RF transmission it may be desirable to maximize the cross-sectional surface area of the antenna to minimize RF energy loss, while simultaneously minimizing PtIr volume, to thereby minimize cost. The thickness of the flat ribbon 902 represents an approximate minimum to maintain antenna shape during assembly, and the height (h) of the flat ribbon is such to achieve necessary surface area.

With reference to FIGS. 11A-11C, the flat end 908 of the antenna 1030 can be electrically coupled to the electronics assembly. To this end, the flat end 908 of the antenna 1030 comprises a first portion 918 separated by a gap from a second portion 920. The first portion 918 includes a first notch 922 at an edge configured to couple with a first feedthrough pin (not illustrated) of the electronics assembly 1010. The second portion 920 includes a second notch 924 at an edge configured to couple with a second feedthrough pin (not illustrated) of the electronics assembly.

Regarding the material and surface finish of the loop antenna 1030 in some embodiments, the antenna is formed of a material comprising platinum (Pt) with an atomic percentage in a range of 70% to 100%, and iridium (Ir) with an atomic percentage in a range of 0% to 30%. In one example configuration, the antenna 1030 is formed of Pt90Ir 10. Platinum and Pt-Ir alloys are selected for a combination of biocompatibility, ductility, and electrical conductivity.

In some embodiments the major surfaces 914 of the antenna 1030 have a surface finish in a range of 0 micro inches and 15 micro inches maximum. In one example configuration, the surface finish is 6 micro inches maximum.

As described above, in configurations of IRPs the antenna 1030 is coupled to the electronics assembly through a dielectric feedthrough, with a dielectric PEEK cover and a backfill encasing the antenna. The backfill may be silicone or a medical grade epoxy adhesive. Encasing an antenna in dielectrics, coupled with post-implant placement of the antenna in boney tissue and muscle may affect antenna performance. However, the antenna 1030, geometry, orientation, material composition, surface finish, etc., disclosed herein in combination with circuitry of the electronics assembly enables post-implant communication at both 2.45GHz and a medical implant communications system (MICS) frequency band, e.g., 401-406MHz, despite the surrounding presence of dielectrics and tissue.

In some embodiments, a loop antenna 1030 having the physical properties described above with reference to FIGS. 11A-11E is tuned to enable post-implant reception of an ultra-low power wakeup sniff signal on a 2.45 GHz channel, while also being tuned to enable post-implant reception and transmission of data and information on a MICS channel, e.g., a 400 MHz channel.

### Conformal Antenna

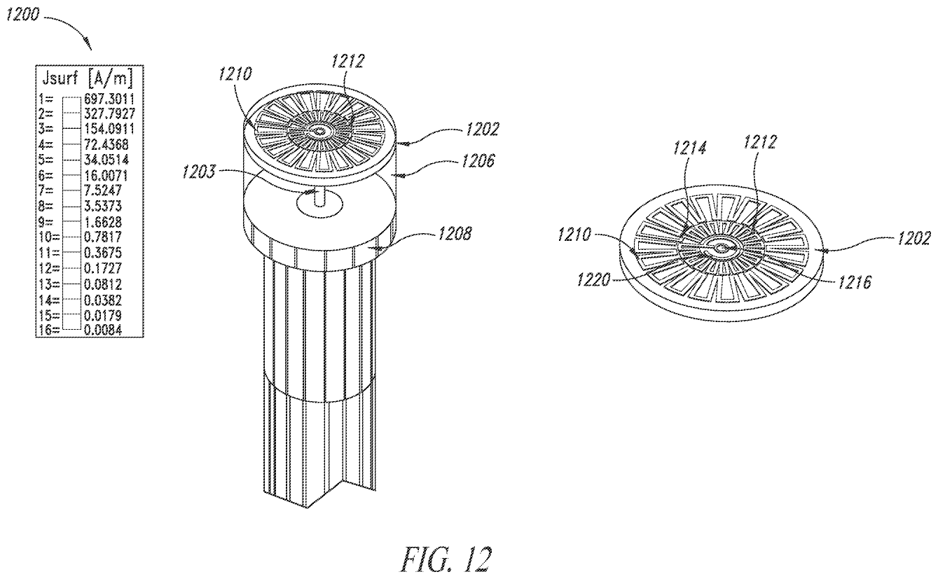

In some embodiments, the antenna 1030 can be a conformal antenna. As illustrated in FIG. 12, a conformal antenna 1200 can be used with the IRP. The conformal antenna 1200 can include conductive traces printed, painted, or otherwise positioned on a substrate 1202. The substrate 1202 can be made of non-conductive biocompatible material, such as, liquid crystal polymer (LCP), polyimide, or polyamide. In some cases, another layer (or seed layer) can be positioned on the substrate 1202 to facilitate adhesion of the conductive traces. Such layer can be made of titanium. The substrate 1202 can be supported by a spacer 1206 supported by a base 1208. A feed 1203 can connect the conformal antenna 1200 to electronic circuitry (not shown), which can include a transmission and receive circuitry. An antenna impedance matching circuitry can connect the antenna feed 1203 and a transceiver. The conformal antenna 1200 can be referred to as a single-layer conformal antenna since the conductive traces are positioned only on one layer of the substrate 1202. In some implementations, as described herein, a conformal antenna can include conductive traces positioned on multiple layers of the substrate 1202 (such as, the top layer and the bottom layer).

The conductive traces can be arranged into a set of outer traces 1210 and a set of inner traces 1212. The two sets of traces 1210 and 1212 can be connected at 1214. The set of inner traces 1212 can be connected to the feed 1203 at a feed point 1216 positioned at the center of the circle. The set of outer traces (or outer ring) 1210 can include plurality of sections shaped as petals, which can be connected to one another. Similarly, the set of inner traces (or inner ring) 1212 can include a plurality of sections shaped as petals, which can be connected to one another. Shaping the traces as petals can result in a design in which traces are symmetrically arranged around the feed point 1216, which has been found to improve the properties of the conformal antenna 1200. Spacing of the petals in one or more sets of traces 1210 or 1212 can affect the resonance of the conformal antenna 1200. For instance, positioning the petals closer together can improve resonance of the conformal antenna 1200 in one or more of the MICS or ISM bands. Adding more petals to the one or more sets of traces 1210 or 1212 can increase the electrical length of the conformal antenna 1200 and improve resonance in one or more of the MICS or ISM bands. Rounding the corners, such as corners that are shaped like a “V,” can affect the resonance of the conformal antenna 1200. Although petal-shaped sections are illustrated in FIG. 12, other symmetrical (or non-symmetrical) shapes can be used. For example, a zig-zag pattern of traces can be used.

The conformal antenna 1200 can include a stub 1220, which can be a round trace connected to the set of inner traces 1212. As described herein, varying the length of the stub 1220 can improve the performance of the conformal antenna 1200.

Although the conformal antenna 1200 is illustrated as circular structure, the antenna may be non-circular in some implementations.

### PIFA Antenna

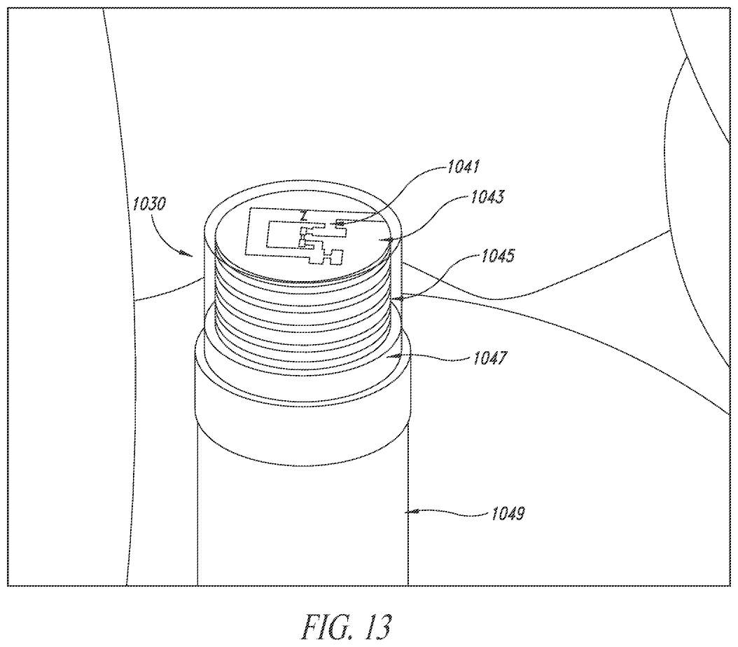

In some embodiments, the antenna 1030 can be a PIFA antenna. FIG. 13 depicts a PIFA antenna supported by a substrate 1043. The PIFA antenna can include conductive traces 1041 printed, painted, or otherwise positioned on the substrate 1043. Conductive traces 1041 can be made of material (such as, one or more of gold, silver, platinum, graphite, copper, etc.) that can be printed, painted, or otherwise positioning on the substrate 1043. The material can be biocompatible. In some cases, the material can be conductive ink. The PIFA antenna can be a type of conformal antenna.

The substrate 1043 can be made of non-conductive biocompatible material, such as, liquid crystal polymer (LCP), polyimide, or polyamide, on which conductive traces can be printed, painted, or otherwise positioned. The material can have long-term biocompatibility. The substrate 1043 can be supported by a spacer 1045, which can separate the traces 1041 from the housing 1049 of the sensor assembly. The spacer 1045 can be made of non-conductive material (such as, thermoplastic) and can serve as a separator between conductive material of the housing 1049 and the conductive antenna traces to improve the antenna performance. As described herein, varying the height of the spacer 1045 can affect the properties of the antenna 1030. To increase the electrical length of the PIFA antenna 1030 (so that antenna can resonate in one or more desired frequency bands), antenna traces can be wound around a spacer 1045 as illustrated in FIG. 13. The spacer 1045 can be supported by a base 1047. A feed (not illustrated) can connect the PIFA antenna 1030 to electronic circuitry (not illustrated), which can include a transmission and receive circuitry. An antenna impedance matching circuitry can connect the antenna feed and a transceiver. Although the PIFA antenna 1030 is illustrated as a circular structure, the antenna may be non-circular in some implementations.

### Helix Antenna

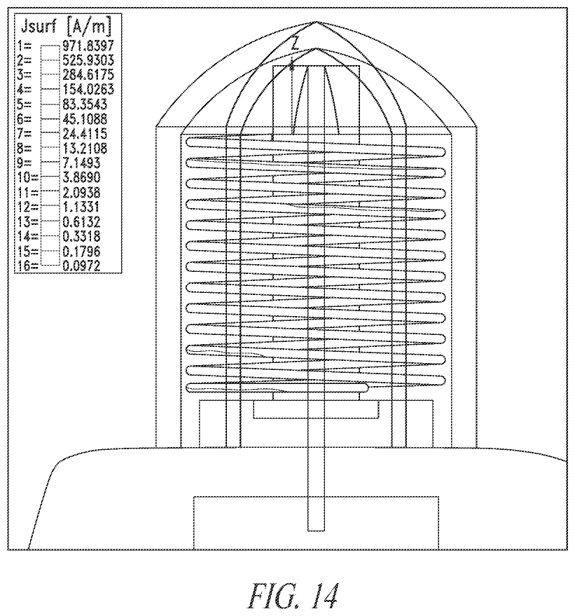

In some embodiments, the antenna 1030 can be a helix antenna. FIG. 14 illustrates an embodiment of a helix or helical antenna.

### Clock and Power Management Circuit

Referring to FIGS. 8 and 9, the clock and power management circuit 1020 can be configured to generate a clock signal for one or more of the other components of the electronics assembly 1010 and can be configured to generate periodic commands or other signals (e.g., interrupt requests) in response to which the controller 1032 causes one or more components of the implantable circuit to enter or to exit a sleep, or other low-power, mode. The clock and power management circuit 1020 also can be configured to regulate the voltage from the battery 1012, and to provide a regulate power-supply voltage to some or all the other components of the electronics assembly 1010.

### Memory

Referring to FIGS. 8 and 9, the memory 1024 can be any suitable nonvolatile memory, such as EEPROM or FLASH memory, and can be configured to store data written by the controller 1032, and to provide data in response to a read command from the control circuit.

### Switch

Referring to FIGS. 8 and 9, the switch 1016 can configured to couple the battery 1012 to, or to uncouple the battery from, the one or more measurement units 1022 in response to a control signal from the controller 1032. For example, the controller 1032 may be configured to generate the control signal having an open state that causes the switch 1016 to open, and, therefore, to uncouple power from the one or more measurement units 1022, during a sleep mode or other low-power mode to save power, and, therefore, to extend the life of the battery 1012. Likewise, the controller 1032 also may be configured to generate the control signal having a closed state that causes the switch 1016 to close, and therefore, to couple power to the one or more measurement units 1022, upon “awakening” from a sleep mode or otherwise exiting another low-power mode. Such a low-power mode may be for only the one or more measurement units 1022 or for the measurement units and one or more other components of the electronics assembly 1010.

The switch 1018 can be configured to couple the battery 1012 to, or to uncouple the battery from, the memory 1024 in response to a control signal from the controller 1032. For example, the controller 1032 may be configured to generate the control signal having an open state that causes the switch 1018 to open, and, therefore, to uncouple power from the memory 1024, during a sleep mode or other low-power mode to save power, and, therefore, to extend the life of the battery 1012. Likewise, the controller 1032 also may be configured to generate the control signal having a closed state that causes the switch 1018 to close, and therefore, to couple power to the memory 1024, upon “awakening” from a sleep mode or otherwise exiting another low-power mode. Such a low-power mode may be for only the memory 1024 or for the memory and one or more other components of the electronics assembly 1010.

### Power Source

### Battery

The intelligent implant will optionally have a power source needed to run the electronics inside the IRP that measures, records and transmits data concerning the state of the implant. Some medical implants already have a power supply. In some embodiments, this power supply is in the form of a battery.

The power profile of the electronic circuitry of the implantable reporting processor can be configured so that the battery has a desired anticipated lifetime suitable for the type of prosthesis (or other device) with which the battery is associated. For example, such a desired anticipated lifetime may range from 1 to 15+ years, e.g., 10 years. In some embodiments, the battery is configured to power the electronic circuitry of the IRP over the entirety (e.g., 18+ years) or the anticipated lifetime of the IPR. An embodiment of such circuitry includes a supply node configured to be coupled to a battery, at least one peripheral circuit, a processing circuit coupled to the supply node and configured to couple at least one peripheral circuit to the supply node, and a timing circuit coupled to the supply node and configured to activate the processing circuit at a set time or set times.

With its LiCFx chemistry, the battery can provide, over its lifetime, about 360 milliampere hours (mAh) at 3.7 volts (V), although one can increase this output by about 36 mAh for each 5 mm of length added to the battery (similarly, one can decrease this output by about 36 mAh for each 5 mm of length subtracted from the battery). It is understood that other battery chemistries can be used if they can achieve the appropriate power requirements for a given application subject to the size and longevity requirements of the application. Some additional potential battery chemistries include, but are not limited to, Lithium ion (Li-ion), Lithium Manganese dioxide (Li-MnO2), silver vanadium oxide (SVO), Lithium Thionyl Chloride (Li-SOCl2), Lithium iodine, and hybrid types consisting of combinations of the above chemistries such as CFx-SVO.

Unfortunately, removing a prosthesis to replace a battery is often undesirable, at least because it involves an invasive procedure that can be relatively expensive and that can have adverse side effects, such as infection and soreness. Thus, the implantable reporting process (IRP) may contain a power source (e.g., a battery) as well as mechanisms to manage the power output of an implanted power source, so that the power source will provide power for a sufficient period of time regardless of the location of the power source within a body of a patient. The IRP may contain the only power source present in the intelligent implant.

The battery can be disposed directly in the prosthesis or can be configured for disposal in a portion of the implantable reporting processor. Or the battery can be configured for disposal in a region of a living body other than the intelligent implant.

### Extending Battery Life

The intelligent implant 1002 can operate in various modes to detect different types of movements. In this way, when a predetermined type of movement is detected, the intelligent implant 1002 can increase, decrease, or otherwise control the amount and type of kinematic data and other data that is collected.

In one example, the intelligent implant 1002 may determine if the patient is moving. The implantable device 1002 can, upon determination that movement has occurred for 10 seconds, begin to store data into memory. In response to the determination, the amount and type of data collected can be started, stopped, increased, decreased, or otherwise suitably controlled. The intelligent implant 1002 may further control the data collection based on certain conditions, such as when the patient stops moving, when a selected maximum amount of data is collected for that collection session, when the intelligent implant 1002 times out, or based on other conditions. After data is collected in a particular session, the intelligent implant 1002 may stop collecting data until the next day, the next time the patient is moving, after previously collected data is offloaded (e.g., by transmitting the collected data to the home base station 1004), or in accordance with one or more other conditions.

In some embodiments, referencing FIG. 9, an IRP 1003 of an intelligent implant may be configured to be placed in different modes of operation. These modes can include, for example: deep-sleep mode, standby mode, low-resolution mode, medium-resolution mode, and/or high-resolution mode.

Deep sleep mode. During deep sleep mode, the IRP 1003 is in an ultra-low power state during storage to preserve shelf life prior to implantation. In this mode, only the clock and power management circuit 1020 circuit and the RF transceiver 1026 wake-up circuitry are active. To this end and with reference to FIG. 9, the battery 1012 provides power to the clock and power management circuit 1020 and the RF transceiver 1026 and the switches 1016, 1017, 1018 are open to decouple the battery 1012 from the IMU 1022, the accelerometer 1023 and the memory 1024.

Standby mode. During stand-by mode, the IRP 1003 can be placed into a low power state, during which the implant is ready for wireless communications with an external device.

Low-resolution mode. During low-resolution mode, the IRP 1003 collects low resolution linear acceleration data for detecting and counting simple motion events and detection of significant motion. In some embodiments, the low-resolution mode is characterized by activation of a first set of sensors, (e.g., the discrete accelerometer 1023 or one or more accelerometers of the IMU 1022) that enable the detection of simple motion events using a sampling rate in the range of 12Hz to 100Hz. To this end and with reference to FIG. 9, one of switches 1016, 1017 is closed to couple the battery 1012 to the IMU 1022 or the discrete accelerometer 1023. When in low-resolution mode, the first set of sensors counts simple motion events and sends significant motion notifications to the controller 1032. In some examples, when exiting the low-resolution mode, the IMU 1022 or discrete accelerometer 1023 reports a count of simple motion events to the controller 1032. The low-resolution mode may be entered on a scheduled time and exited on a schedule time in accordance with a sampling schedule. During low-resolution mode data is continuously collected by the first set of sensors.

Medium-resolution mode. While in medium-resolution mode, the IRP 1003 collects both linear acceleration and rotational motion data. In some embodiments, the medium-resolution mode is characterized by activation of a second set of sensors, (e.g., three accelerometers and three gyroscopes of an IMU 1022) that enable the detection of linear acceleration and rotational velocity using a sampling rate in the range of 12 Hz to 100 Hz. To this end and with reference to FIG. 9, the switch 1016 is closed to couple the battery 1012 to the IMU 1022. In some examples, the medium-resolution mode may be initiated when an unspecified detection of a significant motion event occurs during a configured medium-resolution window of the day, or by a manual command sent wirelessly from an external device, e.g., a base station. The medium-resolution mode may be exited after a pre-determined event related to the detected significant motion.

High-resolution mode. In some embodiments, while in high-resolution mode, the IRP 1003 may collect linear acceleration data, or it may collect both linear acceleration and rotational motion data, or it may collect ultrasound data, or it may collect a combination of all modalities noted. In some embodiments the high-resolution mode is characterized by activation of a third set of sensors (e.g., three accelerometers of an IMU 1022 when collecting only acceleration data or three accelerometers and three gyroscopes of the IMU (when collecting both acceleration and rotational motion data) that enable the detection of acceleration and rotational motion data using a sampling rate in the range of 200 Hz to 5000 Hz. In some embodiments, the high-resolution mode is characterized by the activation of ultrasound sensors to detect implant position using a sampling rate in the 200 Hz to 20,000 Hz range. To this end and with reference to FIG. 9, the switch 1016 is closed to couple the battery 1012 to the IMU 1022. In some examples, the high-resolution mode may be initiated when a specified detection of a significant motion event occurs during a configured medium-resolution window of the day, or by a manual command sent wirelessly from an external device. The high-resolution mode can have a built-in time limit after which acquisition is automatically terminated.

One or more of these modes can be used to collect data passively and autonomously at varying frequencies during the life of the intelligent implant without patient involvement. The intelligent implant may start collecting data on post-operative day 2 and has the capability to store up to 30 days of data in memory. Thereafter, data is transmitted to the cloud daily. In some examples, if the data cannot be transmitted due to connectivity issues with a base station and the implant has reached its memory limit, new data will overwrite the oldest data. Additionally, the base station can store up to 45 days of transmitted data if it is not able to connect to the cloud but is still able to communicate with the implant locally.

In some embodiments, the intelligent implant 1002 can include a fourth set of sensors (e.g., fourth accelerometer on an IMU 1022) that is positioned on a separate board or circuit (not illustrated). In some examples, this separate board or circuit can receive power independent of powering the intelligent implant 1002/IMU 1022 that includes the three sets of sensors. In some embodiments, this allows power to be provided to one accelerometer to detect if walking is occurring rather than having to provide power to the entire intelligent implant 1002/IMU 1022 for the sole purpose of detecting if walking was happening.

### Control Circuit/Controller/Processor

Referring to FIGS. 8 and 9, the controller 1032, which can be any suitable microcontroller or microprocessor, is configured to control the configuration and operation of one or more of the other components of the electronics assembly 1010. For example, the controller 1032 can be configured to control the one or more measurement units 1022 to sense relevant measurement data, to store the measurement data generated by the one or more sensors in the memory 1024, to generate messages include the stored data as a payload, to packetize the messages, to provide the message packets to the RF transceiver 1026 for transmission to the base station (not shown). The controller 1032 also can be configured to execute commands received from a base station (not shown) via the antenna 1030, filter 1028, and RF transceiver 1026. For example, the controller 1032 can be configured to receive configuration data from the base station, and to provide the configuration data to the component of the electronics assembly 1010 to which the base station directed the configuration data. If the base station directed the configuration data to the controller 1032, then the control circuit is configured to configure itself in response to the configuration data.

### Operation of an IRP

Still referring to FIGS. 8 and 9, operation of an implantable reporting processor (IRP) is described in relation to an implanted intelligent implant in which the IRP is disposed, or with which the IRP is otherwise associated.

The fuse 1014, which is normally electrical closed, is configured to open electrically in response to an event that can injure the patient in which the electronics assembly 1010 resides, or damage the battery 1012 of the implantable circuit, if the event persists for more than a safe length of time. An event in response to which the fuse 1014 can open electrically includes an overcurrent condition, an overvoltage condition, an overtemperature condition, an over-current-time condition, and over-voltage-time condition, and an over-temperature-time condition. An overcurrent condition occurs in response to a current through the fuse 1014 exceeding an overcurrent threshold. Likewise, an overvoltage condition occurs in response to a voltage across the fuse 1014 exceeding an overvoltage threshold, and an overtemperature condition occurs in response to a temperature of the fuse exceeding a temperature threshold. An over-current-time condition occurs in response to an integration of a current through the fuse 1014 over a measurement time window (e.g., ten seconds) exceeding a current-time threshold, where the window can “slide” forward in time such that the window always extends from the present time back the length, in units of time, of the window. Alternatively, an over-current-time condition occurs if the current through the fuse 1014 exceeds an overcurrent threshold for more than a threshold time. Similarly, an over-voltage-time condition occurs in response to an integration of a voltage across the fuse 1014 over a measurement time window, and an over-temperature-time condition occurs in response to an integration of a temperature of the fuse over a measurement time window. Alternatively, an over-voltage-time condition occurs if the voltage across the fuse 1014 exceeds an overvoltage threshold for more than a threshold time, and an over-temperature-time condition occurs if a temperature associated with the fuse 1014, battery 1012, or electronics assembly 1010 exceeds an overtemperature threshold for more than a threshold time. But even if the fuse 1014 opens, thus uncoupling power from the electronics assembly 1010, the mechanical and structural components of the intelligent implant (not shown in FIG. 8) are still fully operational.

The controller 1032 can cause the one or more measurement units 1022 to measure movements of the patient and to determine if the measurement is a qualified or valid measurement, to store the data representative of a valid measurement, and to cause the RF transceiver 1026 to transmit the stored data to a base station or other source external to the prosthesis.

Still referring to FIGS. 8 and 9, in response to being polled by a base station (not shown) or by another device external to the implanted device, the controller 1032 can generate conventional messages having payloads and headers. The payloads include the stored samples of the signals that the one or more measurement units 1022 generated, and the headers include the sample partitions in the payload, a time stamp indicating the time at which the measurement unit 1022 acquired the samples, an identifier (e.g., serial number) of the implantable prosthesis, and a patient identifier (e.g., a number or name).

The controller 1032 generates data packets that include the messages according to a conventional data-packetizing protocol. Each packet can also include a packet header that includes, for example, a sequence number of the packet so that the receiving device can order the packets properly even if the packets are transmitted or received out of order.

The controller 1032 encrypts some or all parts of each of the data packets, for example, according to a conventional encryption algorithm, and error encodes the encrypted data packets. For example, the controller 1032 encrypts at least the prosthesis and patient identifiers to render the data packets compliant with the Health Insurance Portability and Accountability Act (HIPAA).

The controller 1032 provides the encrypted and error-encoded data packets to the RF transceiver 1026, which, via the filter 1028 and antenna 1030, transmits the data packets to a destination, such as the base station 1004 (FIG. 7), external to the implantable prosthesis. The RF transceiver 1026 can transmit the data packets according to any suitable data-packet-transmission protocol.

Still referring to FIGS. 8 and 9, alternate embodiments of the electronics assembly 1010 are contemplated. For example, the RF transceiver can perform encryption or error encoding instead of, or complementary to, the controller 1032. Furthermore, one or both of the switches 1016 and 1018 can be omitted from the electronics assembly 1010. Moreover, the electronics assembly 1010 can include components other than those described herein and can omit one or more of the components described herein.

### Base Station

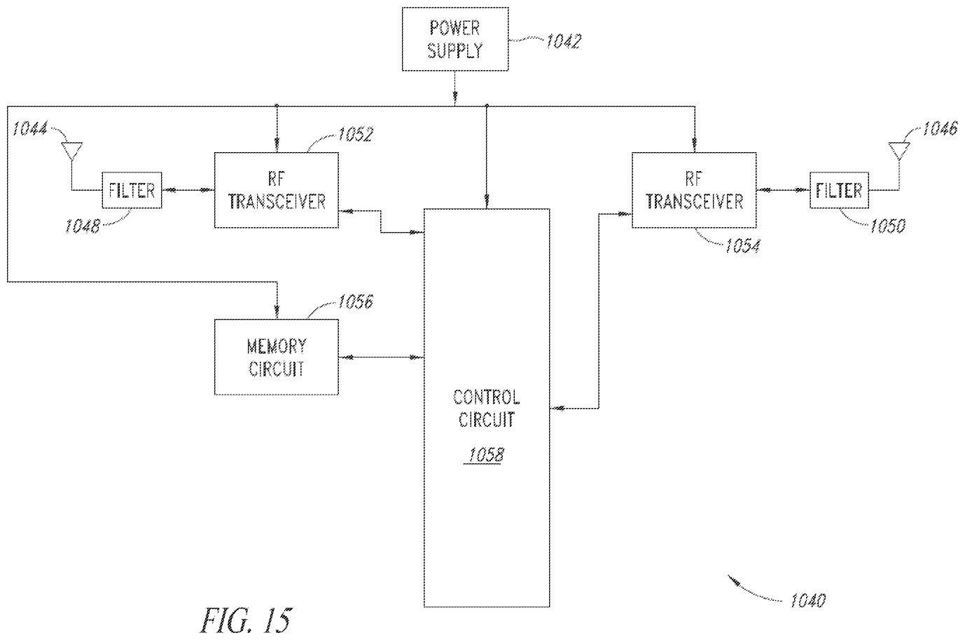

FIG. 15 illustrates an embodiment of a diagram of a base-station circuit 1040, which is configured for inclusion within, or otherwise for use with, a base station, such as the home base station 1004 of FIG. 7, configured for communication with the electronics assembly 1010 of FIG. 8 or 9, according to an embodiment discussed above.

### Configuration

In some embodiments, the base-station circuit 1040 is powered by a power supply 1042, and includes first and second antennas 1044 and 1046, first and second RF filters 1048 and 1050, first and second RF transceivers 1052 and 1054, memory 1056, and a base-station control circuit 1058.

As discussed above, the power supply 1042 can be any suitable power supply, such as a battery or a supply that receives power from an electrical outlet; if the power supply is of the latter type, then the power supply also can include a battery backup for power outages or for while the base-station circuit 1040 is “unplugged.”

The antenna 1044 can be any antenna suitable for the frequency band in which the RF transceiver 1052 communicates with the electronics assembly 1010 of FIG. 8 or 9. Likewise, the antenna 1046 can be any antenna suitable for the frequency band in which the RF transceiver 1054 communicates with a component, e.g., a WiFi® router, access point, or repeater, of the home network 1006 of FIG. 7.

In some embodiments, each of the filters 1048 and 1050 can be any suitable bandpass filter, such as a surface acoustic wave (SAW) filter or a bulk acoustic wave (BAW) filter.

The RF transceiver 1052 can be a transceiver that is configured to allow the control circuit 1058 to communicate with the electronics assembly 1010 of FIG. 8 or 9 while the implant circuit is disposed within or is otherwise associated with an intelligent implant 1002 of FIG. 7. For example, the RF transceiver 1052 can be any suitable type of transceiver (e.g., Bluetooth, Bluetooth Low Energy (BTLE), and WiFi®), can be configured for operation according to any suitable protocol (e.g., MICS, ISM, Bluetooth, Bluetooth Low Energy (BTLE), and WiFi®), and can be configured for operation in a frequency band that is within a range of 1 MHz-5.4 GHz, or that is within any other suitable range.

Likewise, the RF transceiver 1054 can be any transceiver that is configured to allow the control circuit 1058 to communicate with a component, e.g., a WiFi® router, access point, or repeater, of the home network 1006 of FIG. 7, or with one or more of the home base station 1004, the smart device 1005, and the connected personal assistant 1007 of FIG. 7. For example, the RF transceiver 1026 can be any suitable type of transceiver (e.g., Bluetooth, Bluetooth Low Energy (BTLE), and WiFi®), can be configured for operation according to any suitable protocol (e.g., MICS, ISM, Bluetooth, Bluetooth Low Energy (BTLE), and WiFi®), and can be configured for operation in a frequency band that is within a range of 1 MHz-5.4 GHz, or that is within any other suitable range.

The memory 1056 can be any suitable nonvolatile memory, such as EEPROM or FLASH memory, and can be configured to store data written by the control circuit 1058, and to provide data in response to a read command from the control circuit. For example, the control circuit 1058 can store, in the memory 1056, data packets received from the electronics assembly 1010 of FIG. 15 and can store data packets received from a cloud server via the RF transceiver 1054, where the data packets include, for example, commands, instructions, or configuration data for the electronics assembly 1010 of FIG. 8 or 9. Alternatively, the memory 1056 can include volatile memory.

The base-station control circuit 1058, which can be any suitable processor such as a microcontroller or microprocessor, is configured to control the configuration and operation itself and of one or more of the other components of the base-station circuit 1040. For example, the base-station control circuit 1058 can be configured to receive data packets from the electronics assembly 1010 of FIG. 8 or 9 via the RF transceiver 1052, to convert the received data packets into data packets suitable for transmission to the home network 1006 of FIG. 7, and to transmit the converted data packets to the home network via the RF transceiver 1054. And the base-station control circuit 1058 also can be configured to receive data packets from the home network 1006 via the RF transceiver 1054, to convert the received data packets into data packets suitable for transmission to the electronics assembly 1010 of FIG. 8 or 9, and to transmit the converted data packets to the implantable circuit via the RF transceiver 1052.

Still referring to FIG. 15, operation of the base-station circuit 1040 is described in relation to an implanted intelligent device with which the base-station circuit communicates.

The control circuit 1058 polls the electronics assembly 1010 (FIG. 8 or 9) of the implanted intelligent device (not shown) at regular intervals, such as once per day, once every other day, once per week, or once per month. If the control circuit 1058 receives no response to a poll, then the control circuit may poll the electronics assembly 1010 more frequently (e.g., every 5 minutes, every 30 minutes, every hour) until it receives a response or determines that the implanted prosthesis is out of range of the base station.

The electronics assembly 1010 (FIG. 8 or 9) responds to a poll by transmitting all the data packets of samples that the one or more measurement units 1022 of the electronics assembly 1010 generated since the last transmission of data packets.

The antenna RF transceiver 1052 receives the data packets from the electronics assembly 1010 (FIG. 8 or 9) via the antenna 1044 and filter 1048 and provides the received data packets to the base-station control circuit 1058, which decodes and decrypts the data packets, parses the messages from the data packets, and stores the parsed messages in the memory 1056. Before storing the parsed messages, the base-station control circuit 1058 may encrypt part of all of each of the parsed messages for compliance with HIPAA.

Then, the base-station control circuit 1058 reformats the stored messages or generates new messages in response to the headers and payloads of the stored messages. For example, the base-station control circuit 1058 may generate new messages that each include a respective payload and header from a received message, but that each include additional header information such as an identifier of the base station 1004 (FIG. 8 or 9), a time of reception of the original message from the electronics assembly 1010 (FIG. 8 or 9), and time of generation of the new message. Before generating the new messages, the base-station control circuit 1058 may decrypt the parsed messages stored in the memory 1056.

The base-station control circuit 1058 can then generates data packets that include the new messages, encrypts part or all of each of the data packets, and error encodes the data packets, and provides the encrypted and encoded data packets to the RF transceiver 1054, which transmits the encrypted and encoded data packets to the home network 1006 via the filter 1050 and the antenna 1046. The base-station control circuit 1058 may store the encrypted and encoded data packets in the memory 1056 temporarily (e.g., in a buffer) before providing the data packets to the RF transceiver 1054.

In an alternative embodiment, the base-station control circuit 1058 “passes through” the data packets received from the electronics assembly 1010 (FIG. 8 or 9) to the home network 1006 (FIG. 7). That is, the base-station control circuit 1058 receives one or more data packets from the electronics assembly 1010 via the RF transceiver 1052, temporarily stores the one or more data packets in the memory 1056 and causes the RF transceiver 1054 to transmit the one or more data packets to the home network 1006.

In yet another alternative, the control circuit 1058 modifies the one or more data packets received from the electronics assembly 1010 (FIG. 8 or 9) without first parsing the one or more data packets, or with parsing some, but not all, of each data packet.

The home network 1006 (FIG. 7) may “pass through” the one or more data packets received from the base station 1004 to a destination such as a server on the cloud 1008 (FIG. 7) or may modify the one or more data packets according to a suitable communication protocol before sending the one or more data packets to the destination.

Not withstanding above, it is recognized that any device (e.g., smart phones, home computers, tablets, wearable device, etc.) may be used as a home base station to carry out any one or more of the base station functions described herein. For example, the device could be any device having a customized application that allows secure communication, data transfer, storage, and pass through functionality required by the smart implant thereby allowing secure data transfer to the smart implant's Cloud based data storage and analysis system. The device may have compatible wireless communication protocols and circuitry such as but not limited to cellular, Wi-Fi, Zigbee, Bluetooth or BTLF technology.

### Clinical Applications

The following disclosure focuses on spinal fusion by implantation of an interbody spacer or spinal cage; however, this disclosure more generally applies to any of the medical implants as disclosed herein. Currently, post-operative, in-hospital monitoring of spinal fusion surgery is conducted through personal visits by the hospital staff and medical team, physical examination of the patient, medical monitoring (vital signs, etc.), evaluation of range of motion (ROM), physiotherapy (including early mobilization and activity), and diagnostic imaging studies and blood work as required. Once the patient is discharged from hospital, patient satisfaction is checked during periodic doctor's office visits where a thorough history, physical exam and supplemental imaging and diagnostic studies are used to monitor patient progress and identify the development of any potential complications. During such visits, the surgeon typically evaluates the patient's range of motion, attempts to identify any pain that occurs during certain motions or actions, and questions the patient to determine activity levels, daily functioning, pain control, and rehabilitation progress.

Unfortunately, most of the patient's recuperative period occurs between hospital and/or office visits. It can, therefore, be very difficult to accurately measure and follow full range of motion (ROM can change depending on pain control, degree of anti-inflammatory medication, time of day, recent activities, and/or how the patient is feeling at the time of the examination), patient activity levels, exercise tolerance, and the effectiveness of rehabilitation efforts (physiotherapy, medications, etc.) from the day of surgery through to full recovery. For much of this information, the physician is dependent upon patient self-reporting or third party observation to obtain insight into post-operative treatment effectiveness and recovery and rehabilitation progress; in many cases this is further complicated by a patient who is uncertain what to look for, has no knowledge of what “normal/expected” post-operative recovery should be, is non-compliant, or is unable to effectively communicate their symptoms. Furthermore, identifying and tracking complications (in and out of hospital) prior to them becoming symptomatic, arising between doctor visits, or those whose presence is difficult for the patient (and/or the physician) to detect would also provide beneficial, additional information to the management of patient recovery. Currently, in all instances, neither the physician nor the patient has access to the type of “real time,” continuous, objective, prosthesis performance measurements that they might otherwise like to have.

The IMU of the active implant of the present disclosure can provide the surgeon with accurate, numeric, quantitative range of motion data; this data can be compared to expected values to assess efficacy of the procedure post surgery and can serve as a baseline value for comparison to functional values obtained post-operatively. Any abnormalities in vibration (e.g., micromotion which can represent the potential for fusion), rotation (e.g., stiffness of a patient which can identify whether fusion has occurred), and acceleration (e.g., can determine patient movement such as walking speed) can be monitored and the clinician can utilize this data to provide optimal patient care.

Shortly after the spinal fusion and implantation of the lumbar interbody spacer or cage, and following a suitable post-operative recuperating period, the implant begins measuring the movements of the patient to determine quality of life measurements. This can include, for example, step count, cadence, average walking speed, center of mass/angle, and/or bending. The accelerometers can measure the movement and tracking of the spine during movement. As the patient continues to improve their range of motion postoperatively, the acceleration experienced at different locations along the spine, (e.g., the lumbar spine, thoracic spine, the cervical spine) can be monitored. It will be expected that as the patient heals from the surgery, activity levels will progressively increase, and movement will improve and increase. The effects of exercise and various activities can be monitored by the various accelerometers and can be compared to patient's subjective experiences to determine which life activities are improving (or inhibiting) post-operative recovery and rehabilitation.

Integrating the data collected by the sensors described herein (e.g., accelerometers and gyroscopes) with simple, widely available, commercial analytical technologies such as pedometers, allows further clinically important data to be collected such as, but not restricted to: patient activity levels (frequency of activity, duration, intensity), exercise tolerance (work, calories, power, training effect), range of motion (discussed elsewhere herein) and prosthesis performance under various “real world” conditions. It is difficult to overstate the value of this information in enabling better management of the patient's recovery. An attending physician (or physiotherapist, rehabilitation specialist) only observes the patient episodically during scheduled visits; the degree of patient function at the exact moment of examination can be impacted by a multitude of disparate factors such as: the presence or absence of pain, the presence or absence of inflammation, stiffness, time of day, compliance and timing of medication use (pain medications, anti-inflammator recent activity and exercise levels, patient strength, mental status, language barriers, the nature of their doctor-patient relations, or even the patient's ability to accurately articulate their symptoms-to name just a few. Continuous monitoring and data collection can allow the patient and the physician to monitor progress objectively by supplying objective information about patient function under numerous conditions and circumstances, to evaluate how performance has been affected by various interventions (pain control, exercise, physiotherapy, anti-inflammatory medication, rest, etc.), and to compare rehabilitation progress versus previous function and future expected function. Better therapeutic decisions and better patient compliance can be expected when both the doctor and the patient have the benefit of observing the impact of various treatment modalities on patient rehabilitation, activity, function, and overall performance.

The goal of the intelligent implants included herein, is to provide for quality of life improvements of a patient, to address causes of pain (e.g., multi-focal and radicular), and facilitate pain management. The determination of a patient's condition can be done directly and/or indirectly. As discussed above, indirect measurements of a patient's condition can be determined by considering a patient's quality of life. For example, a physician can consider whether a patient's movements increase or decrease over time; whether a patient is able to return to previously enjoyed activities (e.g., exercising, cooking, recreation); whether a patient is able to continue taking care of themselves or maintaining independence; etc. Each of these examples can be determined by the raw data generated by the accelerometers and gyroscopes on the implantable reporting processor. This raw data can be stored and uploaded into the cloud where the data is used to determine a patient's movements that include, for example, step count, cadence, average walking speed, the center of mass of the patient, to name a few.

In some embodiments, as indirect measurements and calculations of kinematic data are done under excitation (e.g., loading and moving), the intelligent implant can determine a patient's baseline movement (e.g., when the patient is static) to use as a normative reference. This normative reference can be used to determine a patient's relative reference (e.g., how quickly a patient returns to regular activities). In some embodiments, the patient's normative reference can be determined by taking a calibration of the patient after the operation (e.g., the spinal fusion) has been complete. In some examples, a demarcation is taken that sets a reference at a known position. This can be, for example, when the patient is against a wall or at a known position (e.g., when the patient's back is straight or at a pre-determined angle).

The intelligent implant, in addition to monitoring a patient's kinematic information, can monitor other data points that can indicate potential patient discomfort or problems during recovery. For example, the sensors within the intelligent implant can monitor a patient's posture. In some embodiments, the intelligent implant can be configured to monitor range of motion (ROM) at the point of fusion (e.g., lumbar ROM, cervical ROM, thoracic ROM). In some examples, the intelligent implant can detect whether a screw of an implant is loosening. As will be discussed in more detail below, the intelligent implant will through direct and indirect measurements-determine whether interbody fusion has occurred, whether the interbody spacer has migrated, and/or whether there is segment subsidence where the intelligent implant is implanted.

In some embodiments, the indirect measurement of patient improvement can be done by measuring the fusion, migration, and subsidence of the intelligent implant. The inertial measurement movement can be used to generate kinematic data to measure the amount of fusion, migration, and/or subsidence of the intelligent implant.

Fusion. As the interbody spacer or spinal cage is press-fit into a space between two adjacent vertebrae, fusion measures the amount of micro-motion in the interbody spacer or spinal cage to determine whether the two adjacent vertebrae have fused. If micro-motion is detected, it indicates to the physician that the patient's vertebrae have not fully fused.

Migration. Migration determines the amount of translation of the interbody spacer or spinal cage positioned between the vertebrae from its initial implantation position. In some embodiments, the inertial measurement unit on the implantable reporting processor can determine the patient's step count, cadence, average walking speed, angle of motion, etc. As discussed above, the increase and/or decrease of migration of the intelligent implant (i.e., the interbody spacer or spinal cage) can indirectly indicate whether a patient's condition is improving. For example, of a patient's movement is decreasing, or there is a change in angle of motion or cadence, this can indicate to a physician that the patient is in pain which can be due to the implant's migration and resulting loss in spacing between the associated vertebral bodies.

Subsidence. Subsidence measures the decrease in vertical height of the vertebral disc space prior to complete fusion. Subsidence is an important consideration as the reduction in disc space between adjacent vertebrae can detrimentally affect mechanical correction and clinical outcomes. As a patient's bone consistency can vary and/or the amount of host site bone preparation by the physician is variable, the amount of potential subsidence experienced by a patient can vary after the spinal fusion has been performed. In some embodiments, changes in a patient's gait detected by the inertial measurement unit may be associated with pain due to a reduction in the intervertebral height and associated nerve compression. This can indicate to a physician that subsidence has occurred. In this case, the physician would call in the patient for direct imaging studies to make a final diagnosis.

In addition to indirect measurements, the intelligent implant presently disclosed can be used to provide direct measurements of migration and subsidence of the interbody spacer or spinal cage. As illustrated in FIGS. 1A-1C, 2A-2D, and 3 provided herein, the implantable reporting processor (e.g., implantable reporting processor 150, 250) can include a plurality of sensors positioned on a top surface, a bottom surface, a medial surface, and a lateral surface to directly measure movement of the interbody spacer or spinal cage.

Migration. As discussed above, migration measures the movement of the interbody spacer or spinal cage. In some embodiments, at least one ultrasound sensor positioned on the medial surface of the cartridge can scan the medial and lateral sides of the interbody spacer or spinal cage to directly measure the amount of movement the interbody spacer or spinal cage is experiencing from its initial implant position by measuring the distance between it and an anatomical reference. In some embodiments, migration of the interbody space or spinal cage can be measure through angular detection (e.g., through a tilt sensor). Migration can be determined by sampling data that measure the position of the interbody spacer or spinal cage when the patient is in a known position. For example, a physician can take a measurement of migration when the patient is sitting in a chair or standing during a visit to the doctor's office. Alternatively, measurements can be taken of the position of the interbody spacer or spinal cage during a time when the patient is in a known position (e.g., at night while the patient is asleep). In some embodiments, a gyroscope can be helpful to determine the orientation of the interbody spacer or spinal cage.

Subsidence. As discussed above, subsidence measures the vertical height of the vertebral disc. In some embodiments, at least one ultrasound sensor positioned on a top surface and a bottom surface of the cartridge can be used to directly measure the position of the bone above and below the interbody spacer or spinal cage. Any changes in the distance between the adjacent vertebrae and a surface of the interbody spacer or spinal cage from the distance measured at the time of implantation can indicate to a physician that the height of the vertebral disc has decreased and subsidence has occurred.

As discussed above, the intelligent implant can be used as a diagnostic tool to help a physician understand the success of spinal fusion. For example, the indirect and direct determinations of fusion, migration, and subsidence of the interbody spacer or spinal cage can provide data to a physician to help diagnose the source of a patient's discomfort or pain after spinal fusion. The information provided by the intelligent implant can help a patient with pain management and/or return to normal activities.

### Disclaimer

The devices, methods, systems etc. of the present disclosure have been described broadly and generically herein. Each of the narrower species and subgeneric groupings falling within the generic disclosure also form part of the present disclosure. This includes the generic description of the devices, methods, systems etc. of the present disclosure with a proviso or negative limitation removing any subject matter from the genus, regardless of whether the excised material is specifically recited herein.

As used herein, the relative terms “medial,” “lateral,” “anterior,” and “posterior” shall be defined from the perspective of device and not necessarily the anatomy. For example, sensors on the “lateral” end could be anatomically facing the posterior direction to detect anterior migration.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the present disclosure are described in terms of Markush groups, it is intended, and those skilled in the art will recognize, that the present disclosure embraces and is also thereby described in terms of any individual member and any subgroup of members of the Markush group, and Applicants reserve the right to revise the application or claims to refer specifically to any individual member or any subgroup of members of the Markush group.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. For example, the term “a sensor” refers to one or more sensors, and the term “a medical device comprising a sensor” is a reference to a medical device that includes at least one sensor. A plurality of sensors refers to more than one sensor. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all the associated items or ideas and one or more other alternative embodiments that include fewer than all the associated items or ideas.

Unless the context requires otherwise, throughout the specification and claims that follow, the word “comprise” and synonyms and variants thereof such as “have” and “include,” as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

Any headings used within this document are only being utilized to expedite its review by the reader, and should not be construed as limiting the disclosure, invention or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the disclosure, invention, or claims. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the term “about” means±20% of the indicated range, value, or structure, unless otherwise indicated.

All the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the present disclosure. The publications discussed above and throughout the text are provided solely for their disclosure prior to the filing date of the present application. Nothing herein is to be construed as an admission that the inventors are not entitled to antedate any referenced publication by virtue of prior invention.

All patents, publications, scientific articles, web sites, and other documents and materials referenced or mentioned herein are indicative of the levels of skill of those skilled in the art to which the disclosure pertains, and each such referenced document and material is hereby incorporated by reference to the same extent as if it had been incorporated by reference in its entirety individually or set forth herein in its entirety. Applicants reserve the right to physically incorporate into this specification all materials and information from any such patents, publications, scientific articles, web sites, electronically available information, and other referenced materials or documents.

In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

As mentioned above, in the following claims, the terms used should not be construed to limit the claims to the specific embodiments disclosed in the specification and the claims but should be construed to include all possible embodiments along with the full scope of equivalents to which such claims are entitled. For example, described embodiments with one or more omitted components or steps can be additional embodiments contemplated and covered by this application.

### EXAMPLE EMBODIMENTS

Embodiment 1: An implantable sensor assembly for use during spinal fusion, the implantable sensor assembly comprising:
a component of an implantable prosthesis; and
an implantable cartridge associated with the component, the implantable cartridge comprising:
   at least one sensor capable of detecting one or more kinematic measurements associated with a patient and generating sensor data, and
   an antenna in electrical communication with at least one sensor, wherein the antenna transmits sensor data to a receiver at a remote location.

Embodiment 2: The implantable sensor assembly of Embodiment 1, wherein the implantable cartridge further comprises a battery.

Embodiment 3: The implantable sensor assembly of Embodiment 1, wherein the implantable cartridge further comprises an inertial measurement unit having a plurality of accelerometers and/or a plurality of gyroscopes.

Embodiment 4: The implantable sensor assembly of Embodiment 3, wherein the inertial measurement unit comprises:
a first accelerometer and/or a first gyroscope for measuring data associated with a first measurement axis,
a second accelerometer and/or a second gyroscope for measuring data associated with a second measurement axis, and
a third accelerometer and/or a third gyroscope for measuring data associated with a third measurement axis.

Embodiment 5: The implantable sensor assembly of Embodiment 1, wherein the implantable cartridge has a length in conjunction with a circular, oval, square, or rectangular cross section.

Embodiment 6: The implantable sensor assembly of Embodiment 1, wherein the implantable cartridge has a cross-section with a corner radius associated with a square or rectangular cross-section.

Embodiment 7: The implantable sensor assembly of Embodiment 1, wherein the implantable cartridge is positioned within the component.

Embodiment 8: The implantable sensor assembly of Embodiment 1, wherein the implantable cartridge is insertable into a slot of the component.

Embodiment 9: The implantable sensor assembly of Embodiment 1, wherein the implantable cartridge is mechanically coupled to the component.

Embodiment 10: The implantable sensor assembly of Embodiment 8, wherein the implantable cartridge is reversibly coupled to the component.

Embodiment 11: The implantable sensor assembly of Embodiment 8, wherein the implantable cartridge forms a one-way positive connection with the component.

Embodiment 12: The implantable sensor assembly of Embodiment 8, wherein the implantable cartridge is mechanically coupled to the component using at least one of corresponding snap rings, locks, twists, threads, or chemical adhesives.

Embodiment 13: The implantable sensor assembly of Embodiment 8, wherein the cartridge is press-fit into the component.

Embodiment 14: The implantable sensor assembly of Embodiment 1, wherein the implantable cartridge comprises a plurality of sensors positioned on a top surface of the implantable cartridge.

Embodiment 15: The implantable sensor assembly of Embodiment 14, wherein the implantable cartridge comprises a plurality of sensors positioned on a bottom surface of the implantable cartridge.

Embodiment 16: The implantable sensor assembly of Embodiment 15, wherein the implantable cartridge comprises at least one sensor positioned on a proximal surface of the implantable cartridge.

Embodiment 17: The implantable sensor assembly of Embodiment 16, wherein the implantable cartridge comprises at least one sensor positioned on a distal surface of the implantable cartridge.

Embodiment 18: The implantable sensor assembly of Embodiment 16, wherein the plurality of sensors positioned on the top surface of the implantable cartridge and the plurality of sensors positioned on the bottom surface of the implantable cartridge is configured to measure subsidence of the implantable sensor assembly.

Embodiment 19: The implantable sensor assembly of Embodiment 17, wherein at least one sensor positioned on a proximal surface of the implantable cartridge and a distal surface of the implantable cartridge provide translational and orientation related movements of the implantable sensor assembly.

Embodiment 20: The implantable sensor assembly of Embodiment 14, wherein the plurality of sensors positioned on the top surface of the implantable cartridge are in series.

Embodiment 21: The implantable sensor assembly of Embodiment 15, wherein the plurality of sensors positioned on the bottom surface of the implantable cartridge are in series.

Embodiment 22: The implantable sensor assembly of Embodiment 1, wherein the implantable cartridge comprises a series of three sensors on a top surface of the implantable cartridge, a series of three sensors on a bottom surface of the implantable cartridge, a plurality of sensors on a proximal end of the implantable cartridge, and a plurality of sensors on a distal end of the implantable cartridge.

Embodiment 23: The implantable sensor assembly of Embodiment 1, wherein the antenna extends from a proximal end or a distal end of the implantable cartridge.

Embodiment 24: The implantable sensor assembly of Embodiment 1, wherein the antenna is contained within a gap of the component.

Embodiment 25: The implantable sensor assembly of Embodiment 21, wherein the antenna is contained within the component whose material composition enables signal transmission from the antenna Embodiment 26: The implantable sensor assembly of Embodiment 25, wherein the component comprises PEEK.

Embodiment 27: The implantable sensor assembly of Embodiment 1 further comprising a processor.

Embodiment 28: The implantable sensor assembly of Embodiment 1, wherein the component of an implantable prosthesis is an interbody spacer for use during spinal fusion.

Embodiment 29: The implantable sensor assembly of Embodiment 28, wherein the interbody spacer is a lumbar interbody spacer.

Embodiment 30: The implantable sensor assembly of Embodiment 28, wherein the interbody spacer is a cervical interbody spacer.

Embodiment 31: The implantable sensor assembly of Embodiment 28, wherein the interbody spacer is a thoracic interbody spacer.

Embodiment 32: The implantable sensor assembly of Embodiment 1, wherein at least one sensor is an ultrasonic sensor.

Embodiment 33: The implantable sensor assembly of Embodiment 32, wherein the ultrasonic sensor is an M-mode sensor.

Embodiment 34: The implantable sensor assembly of Embodiment 32, wherein the ultrasonic sensor is a B-mode sensor.

Embodiment 35: The implantable sensor assembly of Embodiment 32, wherein the ultrasonic sensor is a low-power sensor.

Embodiment 36: The implantable sensor assembly of Embodiment 1, wherein the antenna is a loop antenna.

Embodiment 37: The implantable sensor assembly of Embodiment 1, wherein the antenna is a conformal antenna.

Embodiment 38: The implantable sensor assembly of Embodiment 1, wherein the antenna continuously transmits sensor data.

Embodiment 39: The implantable sensor assembly of Embodiment 1, wherein the antenna intermittently transmits sensor data.

Embodiment 40: The implantable sensor assembly of Embodiment 1, wherein at least one sensor continuously detects one or more physiological parameters.

Embodiment 41: The implantable sensor assembly of Embodiment 1, wherein at least one sensor intermittently detects one or more physiological parameters.

Embodiment 42: The implantable sensor assembly of Embodiment 1, further comprises a power source for providing power to at least one sensor.

Embodiment 43: The implantable sensor assembly of Embodiment 42, wherein the power source is rechargeable.

Embodiment 44: The implantable sensor assembly of Embodiment 1, wherein at least one sensor is capable of being powered by a power source outside a body of a patient.

Embodiment 45: The implantable sensor assembly of Embodiment 1, further comprising a memory device for storing data from at least one sensor.

Embodiment 46: The implantable sensor assembly of Embodiment 1, further comprising a memory device with sufficient memory to enable firmware upgrades of the implantable sensor assembly.

Embodiment 47: A spinal implant assembly for use during spinal fusion, the spinal implant assembly comprising:
an interbody spacer; and
a cartridge mechanically coupled to the interbody spacer, the cartridge comprising:
   at least one sensor capable of detecting one or more physiological parameters of a patient and generating sensor data,
   an antenna in electrical communication with the sensor, wherein the antenna provides bi-directional data communication to a receiver at a remote location, and
   wherein the cartridge is insertable into a mating female cavity within the interbody spacer.

Embodiment 48: The spinal implant assembly of Embodiment 47, wherein the interbody spacer further comprises a battery.

Embodiment 49: The spinal implant assembly of Embodiment 47, wherein the interbody spacer further comprises an inertial measurement unit having a plurality of accelerometers and a plurality of gyroscopes.

Embodiment 50: The spinal implant assembly of Embodiment 49 wherein the inertial measurement unit comprises:
a first accelerometer and a first gyroscope for measuring data associated with a first measurement axis,
a second accelerometer and a second gyroscope for measuring data associated with a second measurement axis, and
a third accelerometer and a third gyroscope for measuring data associated with a third measurement axis.

Embodiment 51: The spinal implant assembly of Embodiment 47, wherein the cartridge has a length in conjunction with a circular, oval, square, or rectangular cross section.

Embodiment 52: The spinal implant assembly of Embodiment 47, wherein a cartridge cross-section has a corner radius associated with a square or rectangular cross-section.

Embodiment 53: The spinal implant assembly of Embodiment 47, wherein the cartridge is mechanically coupled to the interbody spacer.

Embodiment 54: The spinal implant assembly of Embodiment 47, wherein the cartridge is reversibly coupled to the interbody spacer.

Embodiment 55: The spinal implant assembly of Embodiment 47, wherein the cartridge forms a one-way positive connection with the interbody spacer.

Embodiment 56: The spinal implant assembly of Embodiment 47, wherein the cartridge is mechanically coupled to the interbody spacer using at least one of corresponding snap rings, locks, twists, threads, or a chemical adhesive.

Embodiment 57: The spinal implant assembly of Embodiment 47, wherein the cartridge is press-fit into the interbody spacer.

Embodiment 58: The spinal implant assembly of Embodiment 47, wherein the cartridge comprises a plurality of sensors positioned on a top surface of the cartridge.

Embodiment 59: The spinal implant assembly of Embodiment 58, wherein the cartridge comprises a plurality of sensors positioned on a bottom surface of the cartridge.

Embodiment 60: The spinal implant assembly of Embodiment 59, wherein the cartridge comprises at least one sensor positioned on a proximal surface of the cartridge.

Embodiment 61: The spinal implant assembly of Embodiment 60, wherein the cartridge comprises at least one sensor positioned on a distal surface of the cartridge.

Embodiment 62: The spinal implant assembly of Embodiment 59, wherein the plurality of sensors positioned on the top surface of the cartridge and the plurality of sensors positioned on the bottom surface of the cartridge is configured to measure subsidence of the interbody spacer.

Embodiment 63: The spinal implant assembly of Embodiment 61, wherein at least one sensor positioned on a proximal surface of the cartridge and a distal surface of the cartridge provide translational and orientation related movements of the interbody spacer.

Embodiment 64: The spinal implant assembly of Embodiment 58, wherein the plurality of sensors positioned on the top surface of the cartridge are in series.

Embodiment 65: The spinal implant assembly of Embodiment 59, wherein the plurality of sensors positioned on the bottom surface of the cartridge are in series.

Embodiment 66: The spinal implant assembly of Embodiment 47, wherein the cartridge comprises a series of three sensors on a top surface of the cartridge, a series of three sensors on a bottom surface of the cartridge, a plurality of sensors on a proximal end of the cartridge, and a plurality of sensors on a distal end of the cartridge.

Embodiment 67: The spinal implant assembly of Embodiment 47, wherein the antenna extends from a proximal end or a distal end of the cartridge.

Embodiment 68: The spinal implant assembly of Embodiment 47 further comprising a processor.

Embodiment 69: The spinal implant assembly of Embodiment 47, wherein the interbody spacer is inserted into a portion of a lumbar spine of a patient.

Embodiment 70: The spinal implant assembly of Embodiment 47, wherein the interbody spacer is inserted into a portion of a cervical spine of a patient.

Embodiment 71: The spinal implant assembly of Embodiment 47, wherein the interbody spacer is inserted into a portion of a thoracic spine of a patient.

Embodiment 72: The spinal implant assembly of Embodiment 47, wherein at least one sensor is an ultrasonic sensor.

Embodiment 73: The spinal implant assembly of Embodiment 72, wherein the ultrasonic sensor is an M-mode sensor.

Embodiment 74: The spinal implant assembly of Embodiment 72, wherein the ultrasonic sensor is a B-mode sensor.

Embodiment 75: The spinal implant assembly of Embodiment 72, wherein the ultrasonic sensor is a low-power sensor.

Embodiment 76: The spinal implant assembly of Embodiment 47, wherein the antenna is a loop antenna.

Embodiment 77: The spinal implant assembly of Embodiment 47, wherein the antenna is a conformal antenna.

Embodiment 78: The spinal implant assembly of Embodiment 47, wherein the antenna continuously transmits sensor data.

Embodiment 79: The spinal implant assembly of Embodiment 47, wherein the antenna intermittently transmits sensor data.

Embodiment 80: The spinal implant assembly of Embodiment 47, wherein at least one sensor continuously detects one or more physiological parameters.

Embodiment 81: The spinal implant assembly of Embodiment 47, wherein at least one sensor intermittently detects one or more physiological parameters.

Embodiment 82: The spinal implant assembly of Embodiment 47, further comprises a power source for providing power to at least one sensor.

Embodiment 83: The spinal implant assembly of Embodiment 82, wherein the power source is rechargeable.

Embodiment 84: The spinal implant assembly of Embodiment 47, wherein at least one sensor is capable of being powered by a power source outside a body of a patient.

Embodiment 85: The spinal implant assembly of Embodiment 47, further comprising a memory device for storing data from at least one sensor.

Embodiment 86: A method of sampling data from an implantable cartridge coupled to an interbody spacer implanted in a patient, the method comprising:
detecting one or more kinematic measurements associated with a patient's movements and generating sensor data; and
transmitting sensor data to and receiving data from a receiver at a remote location.
Embodiment 87: The method of Embodiment 86, wherein detecting one or more kinematic measurements occurs during movement of the patient.

Embodiment 88: The method of Embodiment 86, wherein detecting one or more kinematic measurements occurs wherein the interbody spacer is under load.

Embodiment 89: The method of Embodiment 86, wherein the method further comprises calibrating the implantable cartridge when the patient is at a known position.

Embodiment 90: The method of Embodiment 89, wherein the known position is when the patient is laying down.

Embodiment 91: The method of Embodiment 89, wherein the known position is when the patient is standing against a wall.

Embodiment 92: The method of Embodiment 89, wherein the known position is when a patient's back is at a predetermined angle while the patient is in a sitting position.

Embodiment 93: The method of Embodiment 92, wherein the predetermined angle is 30 degrees, 45 degrees, or 90 degrees.

Embodiment 94: The method of Embodiment 87, wherein the one or more kinematic measurements are used to determine fusion of the interbody spacer.

Embodiment 95: The method of Embodiment 87, wherein the one or more kinematic measurements are used to determine subsidence of the interbody spacer.

Embodiment 96: The method of Embodiment 95, wherein subsidence measures an amount of force being applied by vertebrae adjacent to the interbody spacer.

Embodiment 97: The method of Embodiment 87, wherein the one or more kinematic measurements are used to determine migration of the interbody spacer.

Embodiment 98: The method of Embodiment 97, wherein migration measures translation of the interbody spacer at a point of implantation.

Embodiment 99: The method of Embodiment 86, wherein migration measures changes in kinematics of the interbody spacer at a point of implantation.

Embodiment 100: The method of Embodiment 86, wherein the one or more kinematic measurements are used to determine patient movement.

Embodiment 101: The method of Embodiment 98, wherein a determined patient movement is at least one of step count, cadence, walking speed, angle of motion, and gait.

Embodiment 102: The method of Embodiment 86, further comprising determining how quickly the patient can return to regular activities.

Embodiment 103: The method of Embodiment 86, wherein the implantable cartridge comprises:
at least one sensor capable of detecting one or more kinematic measurements associated with a patient and generating sensor data, and
an antenna in electrical communication with at least one sensor, wherein the antenna transmits sensor data to and receives data from a receiver at a remote location.

Embodiment 104: The method of Embodiment 101, wherein the implantable cartridge further comprises a battery.

Embodiment 105: The method of Embodiment 86, wherein detecting one or more kinematic measurements comprising obtaining the one or more kinematic measurements from an inertial measurement unit having a plurality of accelerometers and/or a plurality of gyroscopes.

Embodiment 106: The method of Embodiment 105, further comprising:
measuring data associated with a first measurement axis, wherein the data associated with the first measurement axis is obtained from a first accelerometer and a first gyroscope of the inertial measurement unit;
measuring data associated with a second measurement axis, wherein the data associated with the second measurement axis is obtained from a second accelerometer and a second gyroscope of the inertial measurement unit; and
measuring data associated with a third measurement axis, wherein the data associated with the third measurement axis is obtained from a third accelerometer and a third gyroscope of the inertial measurement unit.
Embodiment 107: A spinal implant assembly for use during spinal fusion, the spinal implant assembly comprising:
a spinal implant comprising:
a body,
an opening on a first end of the body,
a cavity extending through the body of the spinal implant from the opening towards a second end side of the body, and
a cartridge configured to be inserted into the cavity, wherein the cartridge comprises an outer wall configured to house a plurality of components.

Embodiment 108: The spinal implant assembly of Embodiment 107, further comprising a locking structure for securing the spinal implant with the cartridge.

Embodiment 109: The spinal implant assembly of Embodiment 107, wherein at least one of a top surface of the body or a bottom surface of the body comprises a plurality of ridges, wherein the plurality of ridges is configured to improve engagement of the spinal implant with an adjacent vertebrae.

Embodiment 110: The spinal implant assembly of Embodiment 107, further comprising a hole extending from a top surface of the body to a bottom surface of the body, the hole of the spinal implant is configured to be filled with biological or synthetic material to aid in spinal fusion.

Embodiment 111: The spinal implant assembly of Embodiment 107, wherein the cartridge includes a power source.

Embodiment 112: The spinal implant assembly of Embodiment 111, wherein the power source is either a single use or a rechargeable battery.

Embodiment 113: The spinal implant assembly of Embodiment 107, wherein the cartridge includes an antenna.

Embodiment 114: The spinal implant assembly of Embodiment 113, wherein the antenna is positioned on the cartridge within the opening of the spinal implant.

Embodiment 115: The spinal implant assembly of Embodiment 113, wherein the antenna is positioned within the outer wall of the cartridge.

Embodiment 116: The spinal implant assembly of Embodiment 113, wherein the antenna is at least one of a loop antenna and a conformal antenna.

Embodiment 117: The spinal implant assembly of Embodiment 113, wherein the antenna continuously transmits sensor data.

Embodiment 118: The spinal implant assembly of Embodiment 113, wherein the antenna intermittently transmits sensor data.

Embodiment 119: The spinal implant assembly of Embodiment 107, wherein the cartridge comprises a processor.

Embodiment 120: The spinal implant assembly of Embodiment 107, wherein the cartridge comprises at least one sensor.

Embodiment 121: The spinal implant assembly of Embodiment 120, wherein the at least one sensor is an ultrasonic sensor.

Embodiment 122: The spinal implant assembly of Embodiment 121, wherein the ultrasonic sensor is a lower-power sensor.

Embodiment 123: The spinal implant assembly of Embodiment 120, wherein the at least one sensor continuously or intermittently detects one or more physiological parameters.

Embodiment 124: The spinal implant assembly of Embodiment 120, wherein the cartridge comprises a memory device for storing data from the at least one sensor.

Embodiment 125: The spinal implant assembly of Embodiment 107, wherein the cartridge has a length in conjunction with a circular, oval, square, or rectangular cross-section.

Embodiment 126: The spinal implant assembly of Embodiment 107, wherein the outer wall has a wall thickness about 0.5 mm.

Embodiment 127: The spinal implant assembly of Embodiment 107, wherein the outer wall has a wall thickness less than 1 mm.

Embodiment 128: The spinal implant assembly of Embodiment 107, wherein the cartridge has a width to height ratio of 1:2.

Embodiment 129: The spinal implant assembly of Embodiment 107, wherein the cartridge has a width to height ratio of 2:3.

Embodiment 130: The spinal implant assembly of Embodiment 107, wherein the cartridge has a width of 8 mm, a thickness of 4 mm, and a length of 28 mm.

Embodiment 131: The spinal implant assembly of Embodiment 107, wherein the cartridge is reversibly coupled to the spinal implant.

Embodiment 132: The spinal implant assembly of Embodiment 108, wherein the locking structure comprises a locking spring finger configured to deform outward on the spinal implant as the cartridge is inserted and is configured to move back into place once the cartridge is fully inserted.

Embodiment 133: The spinal implant assembly of Embodiment 108, wherein the locking structure comprises a pin positioned on the spinal implant and a locking ledge positioned on the cartridge, wherein the pin on the spinal implant is configured to retain the locking ledge to retain the cartridge after insertion.

Embodiment 134: The spinal implant assembly of Embodiment 133, wherein the locking ledge has a radius of the pin.

Embodiment 135: The spinal implant assembly of Embodiment 108, wherein the locking structure comprises a clip or groove positioned along a length of the cartridge, wherein the clip or groove is configured to retain the cartridge within the spinal implant.

Embodiment 136: The spinal implant assembly of Embodiment 107, wherein the spinal implant is an interbody spacer or a spinal cage.

Embodiment 137: An intelligent implant assembly for implantation within a patient, the intelligent implant assembly comprising:
an implant body comprising:
an opening on a first end of the implant body, and
a cavity extending through the implant body from the opening towards a second side of the implant body;
a cartridge inserted into the intelligent implant, wherein the cartridge is retained within the cavity of the implant body and within an outer perimeter of the implant body, and wherein the cartridge comprises an outer wall configured to house a plurality of components; and
a locking structure for securing the spinal implant with the spinal implant.

Embodiment 138: The intelligent implant assembly of Embodiment 137, wherein at least one of a left side surface of the body or a right side surface of the body comprises a plurality of ridges, wherein the plurality of ridges is configured to improve engagement of the spinal implant with an adjacent vertebrae.

Embodiment 139: The intelligent implant assembly of Embodiment 137, wherein the cartridge includes a power source.

Embodiment 140: The intelligent implant assembly of Embodiment 139, wherein the power source is either a single use or a rechargeable battery.

Embodiment 141: The intelligent implant assembly of Embodiment 137, wherein the cartridge includes an antenna.

Embodiment 142: The intelligent implant assembly of Embodiment 141, wherein the antenna is positioned on the cartridge within the opening of the spinal implant.

Embodiment 143: intelligent implant assembly of Embodiment 141, wherein the antenna is positioned within the outer wall of the cartridge.

Embodiment 144: The intelligent implant assembly of Embodiment 141, wherein the antenna is at least one of a loop antenna and a conformal antenna.

Embodiment 145: The intelligent implant assembly of Embodiment 141, wherein the antenna continuously transmits sensor data.

Embodiment 146: The intelligent implant assembly of Embodiment 141, wherein the antenna intermittently transmits sensor data.

Embodiment 147: The intelligent implant assembly of Embodiment 137, wherein the cartridge comprises a processor.

Embodiment 148: The intelligent implant assembly of Embodiment 137, wherein the cartridge comprises at least one sensor.

Embodiment 149: The intelligent implant assembly of Embodiment 148, wherein the at least one sensor is an ultrasonic sensor.

Embodiment 150: The intelligent implant assembly of Embodiment 149, wherein the ultrasonic sensor is a lower-power sensor.

Embodiment 151: The intelligent implant assembly of Embodiment 148, wherein the at least one sensor continuously or intermittently detects one or more physiological parameters.

Embodiment 152: The intelligent implant assembly of Embodiment 148, wherein the cartridge comprises a memory device for storing data from the at least one sensor.

Embodiment 153: The intelligent implant assembly of Embodiment 137, wherein the cartridge has a length in conjunction with a circular, oval, square, or rectangular cross-section.

Embodiment 154: The intelligent implant assembly of Embodiment 137, wherein the outer wall has a wall thickness about 0.5 mm.

Embodiment 155: The intelligent implant assembly of Embodiment 137, wherein the outer wall has a wall thickness less than 1 mm.

Embodiment 156: The intelligent implant assembly of Embodiment 137, wherein the cartridge has a width to height ratio of 1:2.

Embodiment 157: The intelligent implant assembly of Embodiment 137, wherein the cartridge has a width to height ratio of 2:3.

Embodiment 158: The intelligent implant assembly of Embodiment 137, wherein the cartridge is reversibly coupled to the spinal implant.

Embodiment 159: The intelligent implant assembly of Embodiment 137, wherein the locking structure comprises a locking spring finger configured to deform outward on the spinal implant as the cartridge is inserted and is configured to move back into place once the cartridge is fully inserted.

Embodiment 160: The intelligent implant assembly of Embodiment 137, wherein the locking structure comprises a pin positioned on the spinal implant and a locking ledge positioned on the cartridge, wherein the pin on the spinal implant is configured to retain the locking ledge to retain the cartridge after insertion.

Embodiment 161: The intelligent implant assembly of Embodiment 160, wherein the locking ledge has a radius of the pin.

Embodiment 162: The intelligent implant assembly of Embodiment 137, wherein the locking structure comprises a clip or groove positioned along a length of the cartridge, wherein the clip or groove is configured to retain the cartridge within the spinal implant.

Embodiment 163: A method of monitoring patient recovery after spinal fusion, the method comprising:
providing a spinal implant assembly comprising a spinal implant and a cartridge, the cartridge comprising at least one sensor;
collecting data, by the at least one sensor, indicative of at least one of fusion, subsidence or migration of the spinal implant; and
transmitting the data to a remote location.

Embodiment 164: The method of Embodiment 163, wherein the data comprises kinematic measurements of the patient's movements.

Embodiment 165: The method of any of Embodiment 163, wherein the data is indicative of movement of the spinal implant.

Embodiment 166: The method of any of Embodiment 163, wherein the spinal implant comprises an opening on a first end of the spinal implant and a cavity, wherein the cavity extends through the body from a first end of the spinal implant to a second end of the spinal implant.

Embodiment 167: The method of Embodiment 166, wherein the cartridge is configured to be inserted into the opening of the spinal implant such that the cartridge is secured in the cavity of the spinal implant.

Embodiment 168: The method of Embodiment 163, wherein the cartridge comprises a power source, a memory source, and a processor.

Embodiment 169: The method of Embodiment 163, wherein one of the at least one sensor comprises an accelerometer and/or gyroscope.

Embodiment 170: The method of Embodiment 169, wherein the accelerometer and/or gyroscope is configured to measure at least one of a patient's step count, cadence, walking speed, angle of motion, or gait.

Embodiment 171: The method of Embodiment 163, wherein one of the at least one sensor comprises at least one ultrasound sensor.

Embodiment 175: The method of Embodiment 171, wherein the at least one ultrasound sensor is configured to detect translation of the spinal implant.

Embodiment 173: The method of Embodiment 172, wherein the translation of the spinal implant is configured to measure migration of the spinal implant from a point of patient implantation.

Embodiment 174: The method of Embodiment 171, wherein the at least one ultrasound sensor is configured to measure a distance between a surface of the spinal implant and an adjacent vertebrae.

Embodiment 175: The method of Embodiment 172, wherein a change in distance between the surface of the spinal implant and the adjacent vertebrae is configured to measure subsidence of the spinal implant.

Embodiment 176: The method of Embodiment 163, wherein the at least one sensor comprises at least one vibration sensor.

Embodiment 177: The method of Embodiment 176, wherein the at least one vibration sensor is configured to detect acoustic emissions associated with the spinal implant against an adjacent vertebrae.

Embodiment 178: The method of Embodiment 177, wherein the acoustic emissions are configured to measure fusion of the spinal implant against the adjacent vertebrae.

Embodiment 179: The method of Embodiment 163, wherein the at least one sensor is configured to calibrate the cartridge when the patient is at a known position.

Embodiment 180: The method of Embodiment 163, wherein the spinal implant is an interbody spacer or a spinal cage.

Embodiment 181: A method for of monitoring patient recovery after spinal fusion, the method comprising:
receiving data from a spinal implant assembly;
processing the data to evaluate migration of the spinal implant, subsidence of the spinal implant, and/or fusion of the spinal implant; and
providing an output to a clinician based on the processed data.

Embodiment 182: The method of Embodiment 181, wherein the data comprises kinematic measurements.

Embodiment 183: The method of Embodiment 181, wherein the patient kinematic measurements are associated with at least one of a patient's step count, cadence, walking speed, angle of motion, or gait.

Embodiment 184: The method of Embodiment 181, wherein the data comprises measurements indicative of migration of the spinal implant.

Embodiment 185: The method of Embodiment 181, further comprising determining migration of the spinal implant based on translation of the spinal implant.

Embodiment 186: The method of Embodiment 181, wherein the data comprises measurements indicative of subsidence of the spinal implant.

Embodiment 187: The method of Embodiment 181, further comprising determining subsidence of the spinal implant based on a change in the distance between a surface of the spinal implant and an adjacent vertebrae.

Embodiment 188: The method of Embodiment 181, wherein the data comprises measurements indicative of fusion of the spinal implant with adjacent vertebrae.

1-46. (canceled)

47. A spinal implant assembly for use during spinal fusion, the spinal implant assembly comprising:
an interbody spacer; and
a cartridge mechanically coupled to the interbody spacer, the cartridge comprising:
at least one sensor capable of detecting one or more physiological parameters of a patient and generating sensor data,
an antenna in electrical communication with the at least one sensor, wherein the antenna provides bi-directional data communication to a receiver at a remote location, and
wherein the cartridge is insertable into a mating female cavity within the interbody spacer.

48. The spinal implant assembly of claim 47, wherein the cartridge further comprises a battery.

49. The spinal implant assembly of claim 47, wherein the cartridge further comprises an inertial measurement unit having a plurality of accelerometers and a plurality of gyroscopes.

50. The spinal implant assembly of claim 49 wherein the inertial measurement unit comprises:
a first accelerometer and a first gyroscope for measuring data associated with a first measurement axis,
a second accelerometer and a second gyroscope for measuring data associated with a second measurement axis, and
a third accelerometer and a third gyroscope for measuring data associated with a third measurement axis.

51. The spinal implant assembly of claim 47, wherein the cartridge has a length in conjunction with a circular, oval, square, or rectangular cross section.

52. The spinal implant assembly of claim 47, wherein a cartridge cross-section has a corner radius associated with a square or rectangular cross-section.

53. The spinal implant assembly of claim 47, wherein the cartridge is mechanically coupled to the interbody spacer.

54. The spinal implant assembly of claim 47, wherein the cartridge is reversibly coupled to the interbody spacer.

55. The spinal implant assembly of claim 47, wherein the cartridge forms a one-way positive connection with the interbody spacer.

56. The spinal implant assembly of claim 47, wherein the cartridge is mechanically coupled to the interbody spacer using at least one of corresponding snap rings, locks, twists, threads, or a chemical adhesive.

57. The spinal implant assembly of claim 47, wherein the cartridge is press-fit into the interbody spacer.

58. The spinal implant assembly of claim 47, wherein the cartridge comprises a plurality of sensors positioned on a top surface of the cartridge.

59. The spinal implant assembly of claim 58, wherein the cartridge comprises a plurality of sensors positioned on a bottom surface of the cartridge.

60-63. (canceled)

64. The spinal implant assembly of claim 58, wherein the plurality of sensors positioned on the top surface of the cartridge are in series.

65. The spinal implant assembly of claim 64, further comprising a plurality of sensors positioned in series on a on the bottom surface of the cartridge

66. The spinal implant assembly of claim 47, wherein the cartridge comprises a series of three sensors on a top surface of the cartridge, a series of three sensors on a bottom surface of the cartridge, a plurality of sensors on a proximal end of the cartridge, and a plurality of sensors on a distal end of the cartridge.

67. The spinal implant assembly of claim 47, wherein the antenna extends from a proximal end or a distal end of the cartridge.

68. The spinal implant assembly of claim 47, the cartridge further comprising a processor.

69-71. (canceled)

72. The spinal implant assembly of claim 47, wherein the at least one sensor is comprises an ultrasonic sensor.

73-75. (canceled)

76. The spinal implant assembly of claim 47, wherein the antenna is a loop antenna.

77. The spinal implant assembly of claim 47, wherein the antenna is a conformal antenna.

78-84. (canceled)

85. The spinal implant assembly of claim 47, the cartridge further comprising a memory device for storing data from the at least one sensor.

86-188. (canceled)
